(19) United States
(12) Patent Application Publication
(10) Pub. No.: US 2016/0144031 A1
(43) Pub. Date: May 26, 2016

(54) Photo-Responsive Compounds

(71) Applicant: UNIVERSITY OF NORTH CAROLINA AT CHAPEL HILL, Chapel Hill, NC (US)

(72) Inventors: David S. Lawrence, Chapel Hill, NC (US); Thomas Shell, Goffstown, NH (US)

(21) Appl. No.: 14/779,387

(22) PCT Filed: Apr. 8, 2014
(86) PCT No.: PCT/US14/33337

§ 371 Date: Sep. 23, 2015

Related U.S. Application Data
(60) Provisional application No. 61/809,695, filed on Apr. 8, 2013.

(51) Int. Cl. A61K 41/00; A61K 31/69; A61K 35/18; A61K 31/573; A61K 31/165; A61K 31/519; A61K 47/48; A61K 31/7076

(52) U.S. Cl. CPC A61K 41/0042; A61K 47/48092; A61K 31/69; A61K 31/7076; A61K 31/573; A61K 31/165; A61K 31/519; A61K 35/18; A61K 2035/124

(57) ABSTRACT

The presently-disclosed subject matter provides photo-responsive compounds and methods of use thereof. The photo-responsive compounds comprise a photolabile molecule and a fluorophore appended to the photolabile molecule. The presently-disclosed subject matter further relates to a drug delivery system that uses red blood cells to deliver photo-responsive compounds for the treatment of disease.

### RELATED APPLICATION

This application claims priority from U.S. Provisional Patent Application Ser. No. 61/809,695, which was filed on Apr. 8, 2013, the entire disclosure of which is incorporated herein by this reference.

### GOVERNMENT INTEREST

This subject matter of the present disclosure was made with support from the United States government under Grant No. CA079954 awarded by the National Institutes of Health. The United States government has certain rights in the subject matter disclosed herein.

### TECHNICAL FIELD

The presently-disclosed subject matter relates to photo-responsive compounds. In particular, the presently-disclosed subject matter relates to photo-responsive cobalamins and to methods for using the same.

### BACKGROUND

Light-responsive compounds have gained favor as extraordinarily powerful tools for the spatiotemporal control of biochemical and biological processes. Mechanistically, light is used to mediate bond cleavage, which initiates the conversion of an inactive agent (compound) to a biologically active one. (Lee, H. M., et al., 2009; Brieke, C., et al., 2012. Klán, P., et al., 2013.) Although light responsive reagents have been used to manipulate intracellular biochemical pathways and light-sensitive nanoparticles have been employed to site-selectively deliver cytotoxic agents, the potential of both is limited by the short wavelengths (<450 nm) required for photo-activation. Short wavelengths inflict biological damage and are unable to take advantage of the optical window of certain tissues (e.g., 600-1300 nm). (Tromberg, B. J., et al., 2000.) Furthermore, the narrow wavelength range available for photolysis of current compounds limits the ability to design a family of light-responsive species that can be orthogonally activated at distinct wavelengths. (Goguen, B. N., et al., 2011; Hagen, V., et al., 2005; Kantevari, S., et al., 2010; Menge, C., et al., 2011; Priestman, M. A., et al., 2011.)

As an alternative, current technologies utilize two-photon technology light. Substances activated by two-photon light have been activated by wavelengths well into the long-visible/near-infrared (IR) region. However, biologically useful two-photon caging agents are not available. (Bort, G., et al., 2013) Furthermore, two-photon light is not always ideal for biological applications. Hence, there remains a need for improved photo-responsive compounds.

### BRIEF SUMMARY

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of features.

The presently-disclosed subject matter provides a compound comprising a photolabile molecule and a first active agent, wherein the first active agent comprises a fluorophore and is appended to the photolabile molecule. In some embodiments, at least one bond between the first active agent and the photolabile molecule is broken and/or cleaved when the compound is exposed to light.

In some embodiments, the photolabile molecule of the compound is a cobalamin or a derivative of analogue thereof. In some embodiments, the photolabile molecule is an alkylcobalamin.

In some embodiments, the compound includes a second active agent. In certain embodiments, the second active agent comprises a bioactive agent. In some embodiments, the second active agent comprises a second fluorophore.

In some embodiments, the present disclosure provides that the second active agent is chosen from an enzyme, an organic catalyst, a ribozyme, an organometallic, a protein, a glycoprotein, a peptide, a polyamino acid, an antibody, a nucleic acid, a steroid, an antibiotic, an antiviral, an antimycotic, an anticancer agent, an anti-diabetic agent, an anti-analgesic agent, an antirejection agent, an immunosuppressant, a cytokine, a carbohydrate, an oleophobic, a lipid, an extracellular matrix, a demineralized bone matrix, a pharmaceutical, a chemotherapeutic, a cell, a virus, a virus vector, a prion and/or a combination thereof. In certain embodiments, the second active agent is an anti-rheumatoid arthritis agent.

In some embodiments of the present disclosure, the fluorophore of the compound is appended to at least one of a cobalt of the cobalamin and a ribose 5′-OH of the cobalamin.

Further provided in some embodiments of the present disclosure, is a linker disposed between the photolabile molecule and the first active agent. In some embodiments, the linker comprises an alkyl, an aryl, an amino, a thioether, a carboxamide, an ester, an ether, or a combination thereof. Further, in some embodiments, the linker comprises a propylamine, an ethylenediamine, or a combination or derivative thereof.

In some embodiments, the present disclosure provides a linker disposed between the photolabile molecule and the second active agent.

The presently disclosed subject matter further provides, in some embodiments, light comprising a wavelength of about 500 nm to about 1000 nm. In some embodiments, the light comprises a wavelength of about 1000 nm to about 1300 nm. In some embodiments, the light comprises a wavelength of about 500 to about 1300 nm.

In some embodiments, the compound of the present disclosure further includes a pharmaceutically-acceptable carrier.

Further provided in some embodiments of the present disclosure is a method of treating a disease. The method includes the steps of administering an effective amount of a compound according to the present disclosure to a subject at an administration site, and then exposing the administration site to light. In some embodiments, the method includes administering a compound that contains a cobalamin as the photolabile molecule. In some embodiments, the cobalamin is an alkylcobalamin.

Further provided, in some embodiments, is a method that includes administering a compound that further includes a second active agent. In some embodiments, the second active agent is a bioactive agent. In some embodiments, the second active agent includes a second fluorophore. In some embodiments, the second active agent is chosen from an enzyme, an organic catalyst, a ribozyme, an organometallic, a protein, a glycoprotein, a peptide, a polyamino acid, an antibody, a nucleic acid, a steroid, an antibiotic, an antiviral, an antimycotic, an anticancer agent, an anti-diabetic agent, an anti-analgesic agent, an antirejection agent, an immunosuppressant, a cytokine, a carbohydrate, an oleophobic, a lipid, an extracellular matrix, a demineralized bone matrix, a pharmaceutical, a chemotherapeutic, a cell, a virus, a virus vector, a prion and/or a combination thereof.

In some embodiments, the presently disclosed subject matter provides that a fluorophore used in the method(s) is appended to a cobalt center of the cobalamin, to a ribose 5′-OH of the cobalamin or to a combination thereof.

Further, in some embodiments of the disclosed methods, the compound comprises a linker disposed between the cobalamin and the first active agent. In some embodiments, the linker comprises an alkyl, an aryl, an amino, a thioether, a carboxamide, an ester, an ether, or a combination thereof. In some embodiments, the linker comprises a propylamine, an ethylenediamine, or a combination or derivative thereof.

In some embodiments of the methods of the present disclosure, the light comprises a wavelength of about 500 nm to about 1000 nm. In some embodiments, a wavelength of light is about 1000 nm to about 1300 nm. In some embodiments, a wavelength of light is about 600 nm to about 900 nm.

In some embodiments, the present disclosure provides that the administration site is at, in or near a tumor. In some embodiments, non-limiting examples of the disease to be treated includes at least one of rheumatoid arthritis, cancer, and diabetes.

In some embodiments, the present disclosure provides a method that includes administering a compound via at least one of oral administration, transdermal administration, inhalation, nasal administration, topical administration, intravaginal administration, ophthalmic administration, intramural administration, intracerebral administration, rectal administration, parenteral administration, intravenous administration, intra-arterial administration, intramuscular administration, subcutaneous administration, and any combination thereof.

Further provided, in some embodiments is a compound that including a photolabile molecule, a bioactive agent and a lipid, wherein the bioactive agent and the lipid are appended to the photolabile molecule. In some embodiments, the compound further includes a fluorophore appended to the photolabile molecule. In some embodiments, the photolabile molecule is cobalamin.

Still further, the present disclosure provides a cellular membrane. The cellular membrane comprises at least one membrane layer and at least one compound according to the present disclosure, wherein the compound is incorporated in the at least one membrane layer. In some embodiments, the cellular membrane further comprises a fluorophore, wherein the fluorophore is incorporated in the at least one membrane layer. In some embodiments, the cellular membrane is the membrane of a red blood cell.

In some embodiments of the presently-disclosed subject matter, a drug delivery system is provided. The drug delivery system includes a red blood cell, a first compound comprising a photolabile molecule, a bioactive agent, and/or a lipid, wherein the bioactive agent and the lipid are appended to the photolabile molecule. In some embodiments, the compound is incorporated in a cell membrane of the red blood cell. In some embodiments, the compound of the drug delivery system further includes at least one fluorophore.

In some embodiments of the present disclosure, a method of treating a disease is provided. The method comprises administering at least one of the compound, the cellular membrane, and the drug delivery system described herein, to a subject at an administration site; and then exposing the subject and/or the administration site to light, wherein the light has a particular wavelength as described herein.

In some embodiments, the present disclosure provides a method of treating a disease, wherein the method comprises administering to a subject a compound comprising a first active agent that is appended to a photolabile molecule, wherein at least one bond between the first active agent and the photolabile molecule is broken when the compound is exposed to light having a first wavelength and further wherein at least one additional bond between the first active agent and the photolabile molecule is broken when the compound is exposed to light having a second wavelength. In some embodiments of the disclosed method(s), the compound also comprises a second active agent chosen from a fluorophore, an enzyme, an organic catalyst, a ribozyme, an organometallic, a protein, a glycoprotein, a peptide, a polyamino acid, an antibody, a nucleic acid, a steroid, an antibiotic, an antiviral, an antimycotic, an anticancer agent, an anti-diabetic agent, an anti-analgesic agent, an antirejection agent, an immunosuppressant, a cytokine, a carbohydrate, an oleophobic, a lipid, an extracellular matrix or a component thereof, a demineralized bone matrix, a pharmaceutical, a chemotherapeutic, a cell, a virus, a virus vector, a prion and/or a combination thereof. In certain embodiments, the second active agent may be appended to the photolabile molecule. Further, in some embodiments, at least one bond between the second active agent and the photolabile molecule is broken when the compound is exposed to light comprising a first wavelength and/or at least one bond between the second active agent and the photolabile molecule is broken when the compound is exposed to light comprising a second wavelength. In some embodiments of the disclosed method(s), the light comprises a first and/or second wavelength between about 500 nm and about 1300 nm; between about 500 nm and about 1000 nm; and/or between about 1000 nm and about 1300 nm.

### DESCRIPTION OF THE FIGURES

FIG. 1 presents a chart of fractional cobalamin-conjugate (Xᵢ) as a function of photolysis time. Photolysis (Xe flash lamp) of Cbl-1 (10 μM, squares) and Cbl-2 (10 μM, circles) with a 546±10 nm bandpass filter.

FIG. 2 shows sequential and selective photolysis of four cobalamin-conjugates in terms of photolysis yield of cobalamin-conjugates in a mixture as a function of photolysis wavelength. Sequential illumination [(a) 777 nm→(b) 700 nm→(c) 646 nm→(d) 546 nm] serially photolyzes Cbl-5, Cbl-6, Cbl-3, and Cbl-1, respectively.

FIG. 3 shows a schematic of compartmentalized caging. The cobalamin of Cbl-7 restricts BODIPY® 650, a mitochondria targeted agent, to endosomes (pre-photolysis). 650 nm illumination cleaves the Co-BODIPY® 650 linker, enabling cytotoxic BODIPY® 650 to escape from endosomes (post-photolysis) and accumulate in mitochondria (post-photolysis).

FIG. 4 shows red light induced translocation of BODIPY® 650 in HeLa cells, where (a) shows Cbl-7 before photolysis, (b) shows rhodamine B-dextran endosomal marker, (c) shows an overlay of (a) and (b), (d) shows Cbl-7 after photolysis, (e) shows MitoTracker® Green mitochondria marker, and (f) shows an overlay of (d) and (e). HeLa cells are outlined in (a), (b), and (c) based on transmitted images.

FIG. 5 depicts the structures of alkyl-cobalamins and alkyl-cobalamin-fluorophore conjugates.

FIG. 6 depicts Scheme 51, the structure of a cobalamin-TAMRA conjugate (Cbl-1).

FIG. 7 illustrates Scheme S2, synthesis of β-(3-acetamidopropyl)cobalamin (Cbl-2).

FIG. 8 illustrates Scheme S3, the general synthesis of cobalamin-fluorophore conjugates (Cbl-3, Cbl-4, Cbl-5, Cbl-6, and Cbl-7).

FIG. 9 shows Scheme S4, structures of SulfoCy5, carboxylic acid and BODIPY® 650, carboxylic acid.

FIG. 10 illustrates Scheme S5, synthesis of a coenzyme B₁₂-TAMRA conjugate (AdoCbl-1).

FIG. 11 illustrates Scheme S6, the general synthesis of coenzyme B₁₂-fluorophore conjugates (AdoCbl-2, AdoCbl-3, and AdoCbl-4).

FIG. 12 illustrates Scheme S7, the general photolysis of cobalamin-fluorophore conjugates (Cbl-1, Cbl-3, Cbl-4, Cbl-5, Cbl-6, and Cbl-7).

FIG. 13 illustrates Scheme S8, the photolysis of AdoCbl-fluorophore conjugates (AdoCbl-1, AdoCbl-2, AdoCbl-3, and AdoCbl-4) furnishes hydroxocobalamin-fluorophore (B₁₂ₐ-fluorophore) conjugates and adenosine-1 and adenosine-2.

FIG. 14 illustrates the photoinduced conversion of MeCbl (10 μM, squares) to hydroxocobalamin (circles) using an Xe flash lamp at 546±10 nm. Data are represented as averages with standard errors of three independent assays.

FIG. 15 is a graph illustrating photoinduced conversion of Cbl-1 (10 μM, squares) to hydroxocobalamin (circles) using a Xe flash lamp at 546±10 nm. Data are represented as averages with standard errors of three independent assays.

FIG. 16 is a graph illustrating photoinduced conversion of β-(3-acetamidepropyl)cobalamin (Cbl-2, 10 μM, squares) to hydroxocobalamin (circles) using a Xe flash lamp at 546±10 nm. Data are represented as averages with standard errors of three independent assays.

FIG. 17 is a graph illustrating photoinduced conversion Cbl-3 (10 μM, squares) to hydroxocobalamin (circles) using a Xe flash lamp at 646±10 nm. Data are represented as averages with standard errors of three independent assays.

FIG. 18 is a graph showing photoinduced conversion of Cbl-4 (10 μM, squares) to hydroxocobalamin (circles) using a Xe flash lamp at 730±10 nm. Data are represented as averages with standard errors of three independent assays.

FIG. 19 is a graph illustrating photoinduced conversion of Cbl-5 (10 μM, squares) to hydroxocobalamin (circles) using a Xe flash at 780±10 nm. Data are represented as averages with standard errors of three independent assays.

FIG. 20 is a graph showing fluorescent increase of Cbl-1 (1 μM) photolyzed using a spectrofluorometer by excitation at 546 nm and monitoring the fluorescence emission at 580 nm. Data are represented as averages of three independent assays.

FIG. 21 is a bar graph showing fluorescence increase of Cbl-1 (1 μM) photolyzed using a spectrofluorometer tuned to four different wavelengths (546 nm for 5 min, 646 nm for 5 min, 727 nm for 20 min, and 777 for 10 min). Data are represented as averages with standard errors for three independent assays.

FIG. 22 is a graph showing fluorescent increase of Cbl-3 (1 μM) photolyzed using a spectrofluorometer by excitation at 646 nm and monitoring the fluorescence emission at 662 nm. Data are represented as averages of three independent assays.

FIG. 23 shows fluorescence increase of Cbl-3 (1 μM) photolyzed using a spectrofluorometer tuned to four different wavelengths (546 nm for 5 min, 646 nm for 5 min, 727 nm for 20 min, and 777 for 10 min). Data are represented as averages with standard errors for three independent assays.

FIG. 24 shows fluorescent increase of Cbl-4 (1 μM) photolyzed using a spectrofluorometer by excitation at 727 nm and monitoring the fluorescence emission at 752 nm. Data are represented as averages of three independent assays.

FIG. 25 shows fluorescence increase of Cbl-4 (1 μM) photolyzed using a spectrofluorometer tuned to four different wavelengths (546 nm for 5 min, 646 nm for 5 min, 727 nm for 20 min, and 777 for 10 min). Data are represented as averages with standard errors for three independent assays.

FIG. 26 shows fluorescent increase of Cbl-5 (20 μM) photolyzed using a spectrofluorometer by excitation at 777 nm and monitoring the fluorescence emission at 794 nm. Data are represented as averages of three independent assays.

FIG. 27 shows fluorescence increase of Cbl-5 (20 μM) photolyzed using a spectrofluorometer tuned to four different wavelengths (546 nm for 5 min, 646 nm for 5 min, 727 nm for 20 min, and 777 for 10 min). Data are represented as averages with standard errors for three independent assays.

FIG. 28 shows absorption spectra of Cbl-1 Cbl-3, Cbl-4, Cbl-5, and Cbl-6.

FIG. 29 shows fluorescent increase of Cbl-6 (1 μM) photolyzed using a spectrofluorometer by excitation at 700 nm and monitoring the fluorescence emission at 715 nm. Data are represented as averages of three independent assays.

FIG. 30 shows fluorescence increase of Cbl-6 (1 μM) photolyzed using a spectrofluorometer tuned to four different wavelengths (546 nm for 5 min, 646 nm for 5 min, 710 nm for 3 min, and 777 for 10 min). Data are represented as averages with standard errors for three independent assays.

FIG. 31 shows sequential photolysis of a mixture of Cbl-5, Cbl-6, Cbl-3, and Cbl-1 (25 nM each). Relative fraction of photolysis was determined by comparing fluorescence increases due to sequential exposure to wavelengths [777 nm for 3 min (Cbl-5), 700 nm for 3 min (Cbl-6), 650 nm for 3 min (Cbl-3), and 546 nm for 3 min (Cbl-1)] to a photolysis control solution (546 nm for 25 min).

FIG. 32 shows photoinduced conversion of AdoCbl (10 μM, squares) to hydroxocobalamin (circles) using a Xe flash lamp at 546±10 nm. Data are represented as averages with standard errors of three independent assays.

FIG. 33 shows photoinduced conversion AdoCbl-1 (10 μM, squares) to hydroxocobalamin-TAMRA conjugate (circles) using a Xe flash lamp at 546±10 nm. Data are represented as averages with standard errors of three independent assays.

FIG. 34 shows photolysis of AdoCbl (10 μM, circles) and AdoCbl-1 (10 μM, squares) using a Xe flash lamp at 546±10 nm. Data are represented as averages with standard errors of three independent assays.

FIG. 35 shows fluorescent increase of Cbl-7 solution (100 nM) photolyzed using a spectrofluorometer by excitation at 646 nm and monitoring the fluorescence emission at 660 nm. Data are represented as averages of three independent assays.

FIG. 36 shows fluorescence increase of a Cbl-7 solution (100 nM) photolyzed using a spectrofluorometer tuned to four different wavelengths (546 nm for 5 min, 646 nm for 5 min, 727 nm for 20 min, and 777 for 10 min). Data are represented as averages with standard errors for three independent assays.

FIG. 37 shows fluorescence increase of Cbl-7 in HeLa cells upon photolysis at 650 nm. (a) 0 min (b) 5 min (c) 10 min (d) 15 min. Imaging and photolysis was accomplished utilizing an Olympus IX-81 widefield fluorescence microscope with a Cy5 filter cube.

FIG. 38 shows fluorescence increase of HeLa cells loaded with Cbl-7 as a function of time and imaged using a Cy5 filter cube.

FIG. 39 shows Cbl-7 in HeLa cells is retained by endosomes upon incubation in the dark (5 h). (a) Cbl-7 (500 nM; ex/em 650/665 nm) (b) the endosomal marker Rhodamine B-dextran (1 mg/mL; ex/em 570/590 nm) (c) overlay of (a) and (b). Mander's coefficient=0.81.

FIG. 40 illustrates three representative examples of light-responsive agents: cofilin (3), light-activated protein Kinase C (PKC) sensor (4), and natural product ponasterone (5).

FIG. 41 illustrates photolysis of organocobalamins, including the photosensitivity of coenzyme B₁₂ (6; where R=5′-deoxyadenosyl or H), light induced hemolytic cleavage of the Co³⁺-alkyl bond, initially furnishing Cbl (Co⁺²) 7 and alkyl radical 8 products (Scheme 1).

FIG. 42 illustrates Cbl-fluorophore derivatives undergoing photolysis at the excitation wavelengths of the appended fluorophores, including those containing TAMRA (546 nm, 9/10).

FIG. 43 illustrates the structures of photo-release of bio-Active species from cobalamins: Cbl-BODIPY® 650 11, Cbl-cAMP 12, and Cbl-doxorubicin 13.

FIG. 44 illustrates the Cbl-Cy5 (left) and Cbl-Dylight® 800 (right) derivatives respond orthogonally to 646 and 777 nm, respectively.

FIG. 45 shows the structures of starting fluorophore-substituted conjugates (14) and the photolyzed products (15).

FIG. 46 illustrates the wavelength-dependent photo-release of Cbl derivatives (17 and 21) of methotrexate (16) and dexamethasone (19). Both drugs are routinely used for the treatment of rheumatoid arthritis (RA). The highlighted carboxylate in 16 is not required for activity and a variety of substituents (including peptides, antibodies, and polymers) have been conjugated to this position (Majumdar 2012; Wang 2007; Everts 2002). Most relevant to this discussion are the array of anti-inflammatory N-alkyl carboxamide MTX derivatives that are analogous/identical to the expected photolyzed products (18) (X═H, OH). (Heath 1986; Rosowsky 1986; Piper 1982; Rosowsky 1981; Szeto 1979). DEX (19) is also pharmaceutically available as the acetate (20) (R=Me) that, like many other short chain acylated Dex derivatives (e.g. 22), is designed to promote skin/ocular permeability (Markovic 2012; Civiale 2004) or serve as a sustained release version when injected as an intramuscular depot due to its low water solubility (Samtani 2005).

FIG. 47 shows synthesis of thiolato-Cbls (24) by exposing mercaptans to (23) under neutral, aqueous, aerobic conditions (Scheme 2). Photolysis in air produces the Co(II)-Cbl product, which is oxidized to the Co(III) species, and a thiyl radical, which is converted to a disulfide or oxidized product (Scheme 2). (Tahara, 2013)

FIG. 48 illustrates structure of one of the primary intracellular forms of vitamin B₁₂ glutathione-Cbl (25) and a thiolato-Cbls N-acetylCys 26 and the photolysis in air Co(II)-Cbl product, which is oxidized to the Co(III) species, and a thiyl radical, which is converted to a disulfide or oxidized product (Scheme 2).

FIG. 49 illustrates structures of Cbl-Cys analogs (30-33) of a protein kinase substrate (28).

FIG. 50 shows that lipidated photo-releasable bio-active agents (R) hidden within a protective protein sheath on a cell membrane. (a) Bio-agent R membrane-bound via a single anchor and (b) a double Cys-containing bio-active peptide membrane-bound via a double anchor.

FIG. 51 illustrates structures of RBC membrane embedded/photo-releaseable derivatives (35) and (36).

FIG. 52 illustrates the structure of a lysine derivative (37).

FIG. 53 is a diagram that illustrates leukocyte migration (145) across an endothelial monolayer. Anti-inflamma-tories should block CAM expression, monocyte-EC interactions, and cell migration. Adapted from ref Muller 2008.

FIG. 54 is a diagram that illustrates (a) Lipid-Cbl-spacer-GRGDSY on the surface of RBCs. (b) A high avidity between RBCs and ECs due to multiple interactions between RGD peptides and integrins should block the attempted migration of monocytes.

FIG. 55 illustrates (a) Localized drug (black dots) photo-release from endothelial layer-bound RBCs should enhance drug uptake by the endothelial layer and T cells/synoviocytes in the lower chamber relative to (b) drug photo-release from unattached RBCs.

FIG. 56 shows the structures of drug/fluorophore B₁₂ conjugates.

FIG. 57 shows the structures of flurophore antennas.

FIG. 58 shows the synthesis of membrane anchors.

FIG. 59 shows the synthesis and purification of MTX-C₁₈—B₁₂.

FIG. 60 shows synthesis of monofunctionalized cobalamins.

FIG. 61 shows synthesis of MTX B₁₂ (Cbl-2).

FIG. 62 shows the synthesis of deacetylcolchicine.

FIG. 63 shows the synthesis of colchicine-C₁₈—B₁₂ (Cbl-3).

FIG. 64 shows synthesis of colchicine-B₁₂ (Cbl-4).

FIG. 65 shows synthesis of dexamethasone-C₁₈—B₁₂ (Cbl-5).

FIG. 66 shows synthesis of 5-TAMRA-C₁₈—B₁₂ (Cbl-6).

FIG. 67 shows synthesis of 5-FAM-C₁₈—B₁₂ (Cbl-7).

FIG. 68 shows synthesis of Cy5-C₁₈ (Fl-1). Synthesis of Cy5-C18 (Fl-1) (4) a) Br(CH₂)₅CO₂H, KI, CH₃CN b) CH₃I c) malonaldehyde dianilide, AcOH, Ac₂O d) 2, pyridine, AcOH e) DIC (N,N′-diisopropylcarbodiimide), TEA, octadecylamine, CH₂Cl₂, Cy5 synthesized as previously reported (Kiyose, K.; Hanaoka, K.; Oushiki, D; Nakamura, T.; Kajimura, M.; Suematsu, M.; Nishimatsu, H.; Yamane, T.; Terai, T; Hirata, Y; and Nagano, T. JACS. 2010, 132, 15846-15848.).

FIG. 69 shows synthesis of Cy7-C₁₈ (Fl-2). Synthesis of Cy7-C18 (6) a) N-[5-(Phenylamino)-2,4-pentadienylidene]aniline monohydrochloride, AcOH, Ac₂O, b) 7, AcOH, pyridine c) DIC, TEA, octadecylamine, CH₂Cl₂

FIG. 70 shows Synthesis of Dy800-C₁₈ (Fl-4). Synthesis of Dy800-C₁₈ (12) a) 3-methyl butanone, AcOH; KOH, MeOH, PrOH b) (10): 1,3-propane sultone, o-dichlorobenzene (11): Br(CH₂)₅CO₂H, o-dichlorobenzene c) 3-chloro-2,4-trimethyleneglutacondianil hydrochloride, AcONa, EtOH d) 10 e) sodium phenoxide, DMF f) DIC, DIPEA, octadecylamine, DMF.

FIG. 71 illustrates Cbl-6 and Cbl-7 photocleaved from RBC Membranes. Fluorescein release and TAMRA release from cobalamins (Cbl-7 and Cbl-6, respectively) bound to erythrocytes using 525 nm light.

FIG. 72 illustrates using C₁₈ conjugated fluorophores to extend photocleavage of FAM into the near IR (NIR). Releasing Fluorescein (from Cbl-7) using Fl-1 (650 nm), Fl-2 (700 nm), and Fl-3 (730 nm). Erythrocytes were loaded with 1 μM Cbl-7 and 5 μM Fluorophore-C₁₈. Photolysis was performed using the above mentioned wavelengths of light for 30 min. Notably, cobalamin (aka B₁₂) only absorbs light up to around 550 nm; therefore in order to absorb light beyond this wavelength, the presence of an antenna fluorophore is required.

FIG. 73 illustrates a graph for determining [Cbl-6]:[Fl-1] ratio of optimal release using 650 nm light.

FIG. 74 illustrates an MTX standard curve.

FIG. 75 illustrates photo release of methotrexate (MTX) from erythrocyte membranes. Releasing MTX from red blood cells (RBCs) over time using 525 nm light and 650 nm light. The bars on the left of each pair indicate the presence of 5 μM Fl-1 and 1 μM Cbl-1. The bars on the right of each pair contain only CNA Fl-1 is clearly required for efficient drug release at 650 nm.

FIG. 76 illustrates an MTX DHFR inhibition assay, showing inhibition of DHFR using methotrexate (circles) and photolyzed methotrexate (triangles).

FIG. 77 illustrates a colchicine standard curve.

FIG. 78 illustrates colchicine-c₁₈-b₁₂(cbl-3) octanol/h₂o migration. Photolyzed colchicine (from Cbl-3) diffuses from octanol into water and does so in increasing amounts until maximal photolysis at 10 min. Due to the hydrophobic nature of the molecule, the equilibrium prefers octanol even after cleavage but there is no detectable migration into the water until cleavage occurs.

FIG. 79 illustrates the effect of colchicine on HeLa cells, wherein colchicine works as a positive control. As more colchicine is added, the tubulin networks become disrupted.

FIG. 80 illustrates effects of treatment of HeLa cells with cbl-3 loaded RBCs. a) HeLa cells exposed to Cbl-3 loaded RBCs without photolysis. b) HeLa cells exposed to Cbl-3 loaded RBCs illuminated with 525 nm light for 20 min. c) HeLa cells with no RBC or light exposure. d) HeLa cells without RBCs and with 20 min photolysis at 525 nm.

FIG. 81 illustrates the effects of treatment of HeLa cells with dexamethasone. The effects of dexamethasone on the distribution of GRα. The steroid receptor is evenly distributed in the cystosol in a) due to the absence of dexamethasone. After the addition of 250 nM dexamethasone in b) the receptor migrates to the nucleus and the same is observed in c) with 500 nM dexamethasone.

FIG. 82 illustrates the effects of treatment of HeLa cells with cbl-5 loaded RBCs. These are GRα stained HeLa cells. a) Cbl-5 loaded RBCs without photolysis. b) No RBCs and no photolysis. c) Cbl-5 loaded RBCs exposed to 525 nm light for 20 min. d) No RBCs with 20 min 525 light exposure.

FIG. 83 illustrates the effects of treatment of HeLa cells with cbl-5 loaded RBCs and removal pre-photolysis (leakage test). To determine if Cbl-5 is in an equilibrium with the RBCs and the cell culture, RBCs loaded with Cbl-5 were exposed to HeLa cells in a) and then removed before photolysis. GRα was not affected, indicating that dexamethasone remains on the RBC until photolysis occurs. b) Contains cells that were exposed to Cbl-5 loaded RBCs and then washed with no photolysis. c) Contains HeLa cells that were photolyzed but were not exposed to RBCs.

FIG. 84 illustrates the results of treatment of HeLa cells with cbl-5 loaded RBC at different wavelengths. HeLa cells exposed to Cbl-5 loaded RBCs illuminated at 530 and 780 nm.

FIG. 85 illustrates the results of treatment of HeLa cells with cbl-5 and fl-4 RBCs. 780 nm Release of C₁₈-Dexamethasone-B₁₂/Dylight 800 RBCs

FIG. 86 illustrates results of hemolysis study. Hemolysis was measured at different concentrations of each of the lipophilic drug complexes. The RBCs are stable to loading concentrations at or below 5 μM in each case.

FIG. 87 illustrates mesoporous silica nanoparticles containing drugs in the channels and the channels capped with cobalamin.

FIG. 88 illustrates the fluorophore-Cbls structures capping the channels of mesoporous silica nanoparticles.

FIG. 89 illustrates release of fluorescein from cobalamin capped mesoporous silica nanoparticles (Fl-MSNP). Fluorescence intensity is relative to blank background sample. A sample was stored in the dark (5 h) then subsequently photolyzed (525 nm) for two periods (30 min). The samples were mixed (2.5 h) after each light exposure.

### DETAILED DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Each example is provided by way of explanation of the present disclosure and is not a limitation thereon. In fact, it will be apparent to those skilled in the art that various modifications and variations can be made to the teachings of the present disclosure without departing from the scope of the disclosure. For instance, features illustrated or described as part of one embodiment can be used with another embodiment to yield a still further embodiment.

All references to singular characteristics or limitations of the present disclosure shall include the corresponding plural characteristic(s) or limitation(s) and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods and compositions of the present disclosure, including components thereof, can comprise, consist of, or consist essentially of the essential elements and limitations of the embodiments described herein, as well as any additional or optional components or limitations described herein or otherwise useful.

The presently-disclosed subject matter includes photo-responsive compounds, and in particular, certain embodiments include compounds that comprise cobalt that are appended to a photo-responsive ligand. In some embodiments the compounds of the present disclosure comprise cobalamin. In some embodiments the photo-responsive ligand is a fluorophore.

When the photo-responsive compounds of the present disclosure are exposed to light, at least one bond between the fluorophore and the cobalamin is cleaved. As used herein, the terms “photo-cleavable,” “photo-releasable,” “photo-activated,” “photo-responsive,” and the like are used interchangeably to describe compounds wherein one or more bonds is broken upon that compound's exposure to light.

In certain embodiments, the compounds of the present disclosure comprise structures represented by formula (I), as shown below:

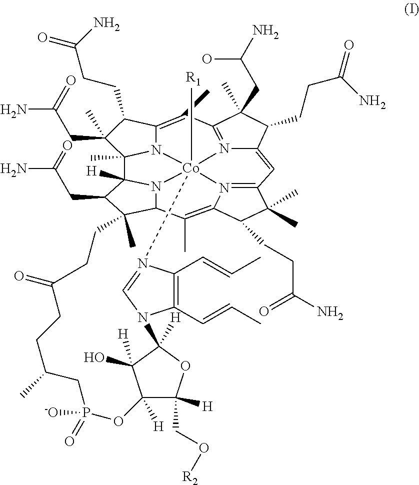

wherein R₁ and R₂ can be the same or different from one another, wherein at least one of R₁ and R₂ comprises a fluorophore, H, and/or alkyl.

In certain embodiments, the compound comprising formula (I) can be described as comprising an active agent (e.g, a cytotoxic species), an enzyme inhibitor, an enzyme activator, and/or a biochemical sensor.

Further, the presently-disclosed subject matter also includes any pharmaceutically acceptable salts or a pharmaceutically acceptable derivatives of the compounds described herein.

As discussed above, in some embodiments the compound(s) of the present disclosure comprise cobalamin. In some embodiments the cobalamin is substituted cobalamin. For example, the cobalamin of the present disclosure can be an alkylcobalamin, such as methylcobalamin. In come embodiments, the compounds of the present disclosure comprise at least one cobaloxime, including substituted cobaloximes, such as alkylcobaloximes.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds, peptides, lipids, oligonucleotides, and oligosaccharides. Illustrative substituents include, for example, those described herein. The permissible substituents can be one or more and the same or different for appropriate organic compounds. For purposes of this disclosure, cobalamin may comprise alkyl substituents and/or any permissible substituents of organic compounds described herein, including those that induce strain in the embodied compounds. This disclosure is not intended to be limited in any manner by the permissible substituents of organic compounds.

With regard to alkyl substituents, the term “alkyl” refers to alkyl groups with the general formula CₙH₂ₙ₊₁, where n is in the range of about 1 to about 18 or more. The groups can be straight-chained or branched. Alkyl, when used herein, also comprises “lower alkyls,” which refer to alkyl groups with the general formula CₙH₂ₙ₊₁, where n is in the range of about 1 to about 6. In some embodiments, n is about 1 to about 3. Examples include methyl, ethyl, propyl, isopropyl, n-butyl, sec-butyl, t-butyl, isobutyl, n-pentyl, isopentyl, neopentyl, n-hexyl, and the like. Throughout the specification “alkyl” is generally used to refer to both unsubstituted alkyl groups and substituted alkyl groups, and this practice holds true for the other groups (e.g., cycloalkyl, etc.) described herein.

Further still, any suitable fluorophore known in the art can be used in embodiments of the presently-disclosed subject matter. The term, “fluorophore,” as used herein refers to a species of compounds that can accept and/or is excited by energy (e.g., light), wherein the fluorophore generates fluorescence when it accepts and/or is excited by energy.

Exemplary fluorophores that can be used in the embodied compounds include alkyl-tetramethyl-rhodamine (e.g., 5-carboxytetramethylrhodamine (TAMRA)), sulfo-Cy5, ATTO 725, Alexa Fluor® 700, BODIPY® 650, 5-Fam, Cy3, Alexa Fluor® 546, Alexa Fluor® 555, Alexa Fluor® 568, Atto 590, DyLight® 594, CF 594, Alexa Fluor® 594, ATTO 610, Alexa Fluor® 610, Texas Red, ATTO 620, CF 620, Red 630, ATTO 633, CF 633, Alex Fluor® 633, DyLight 633, Alexa Fluor® 635, Cy5, CF 640, ATTO 647, Alexa Fluor® 647, CF 647, DyLight® 650, IRDye 650, ATTO 655, Alexa Fluor® 660, CF 660, Alexa Fluor® 680, IRDye 680, Atto 680, DyLight® 680, CF 680, Red 681, Alexa Fluor® 700, Atto 700, IRDye 700, NIR 700, NIR 730, ATTO 740, Alexa Fluor® 750, Cyto 750, CF 750, Cy7, IRDye 750, DyLight 755, Cy7.5, Cyto 770, Alexa Fluor® 790, CF 770, Cyto 780, IRDye 800, DyLight 800, Cyto 840, and the family of quantum dots including Qdots, Trilite Nanocrystals, alloyed Quantum Dots, CdS Type Quantum dots, Cd Se Type Quantum dots, Core-Shell Type Quantum Dots, or combinations thereof.

Additionally, exemplary fluorophores that can be used in embodied compounds include Alexa Fluor® 610, Alexa Fluor® 633, Alexa Fluor® 647, Alexa Fluor® 660, Alexa Fluor® 680, Alexa Fluor® 700, Alexa Fluor® 750, BODIPY® FL, BODIPY® TMR, BODIPY® 493/503, BODIPY® 499/508, BODIPY® 507/545, BODIPY® 530/550, BODIPY® 577/618, BODIPY® 581/591, BODIPY® 630/650, BODIPY® 650/665, Cy-2, Cy-3, Cy-5, Cy-7, Eosin, Fluo-4, Fluorescein, Lucifer yellow, NBD, Oregon Green® 488, PyMPO, Rhodamine Red, Sulfonerhodamine, Tetramethylrhodamine, and/or Texas Red®.

In some embodiments, “fluorophore” includes a molecule that absorbs light energy of a certain wavelength, including, e.g., violet, blue, cyan, green, yellow-green, yellow, orange, red-orange, red, far-red, near infrared, or infrared, and emits light energy of a different wavelength, and the term encompasses those molecules that emit in a variety of spectra, for example, including violet, blue, cyan, green, yellow-green, yellow, orange, red-orange, red, far-red and/or infrared.

In an embodiment, a fluorophore is a violet fluorescent dye, a blue fluorescent dye, a cyan fluorescent dye, a green fluorescent dye, a yellow-green fluorescent dye, a yellow fluorescent dye, an orange fluorescent dye, a red-orange fluorescent dye, a red fluorescent dye, a far-red fluorescent dye, a near infrared fluorescent dye or an infrared fluorescent dye. Non-limiting examples of a fluorescent dye include dyes derived from, e.g., a coumarin, a cyanine, a fluorescein, an isocyanate, isothiocyanate, an indocarbocyanine, an indodicarbocyanine, a pyridyloxazole, a phycoerythrin, a phycocyanin, an o-phthaldehyde and a rhodamine.

The fluorophore, and optionally other molecules, can be appended to the compound at various points. For instance, in embodiments that comprise cobalamin, the fluorophore can be appended either directly or via a linker to the cobalt center of the cobalamin, to a ribose 5′-OH of the cobalamin, to other locations on the cobalamin, or to combinations thereof. Similarly, in embodiments that comprise cobaloximes, the fluorophore can be appended, directly or via a linker, to the cobalt center of the cobaloxime.

In certain embodiments, the linker between the compound and the fluorophore, or any other appended molecule, can be any suitable molecule that can conjugate two or more molecules.

For example, in some embodiments the linker is an alkyl, an aryl, an amino, a thioether, a carboxamide, an ester, an ether, and/or a combination thereof. Those of ordinary skill in the art will appreciate other linkers that can be used in certain embodiments of the presently-disclosed subject matter. The linker can thus be any atom or molecule that is bound (e.g., covalently bound) both to the compound and/or to the fluorophore. Exemplary linkers include propylamine, ethylenediamine, or combinations or derivatives thereof.

The term “aryl” as used herein is a group that contains any carbon-based aromatic group including, but not limited to, benzene, naphthalene, phenyl, biphenyl, phenoxybenzene, and the like. The term “aryl” also includes biaryls (e.g., naphthalene or biphenyl) or “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “ester” as used herein is represented by a formula —OC(O)A¹ or —C(O)OA¹, where A¹ can be an optionally substituted alkyl, cycloalkyl, aryl, or the like. The term is inclusive of “polyester,” which, as used herein, is represented by a formula -(A¹O(O)C-A²-C(O)O)ₐ— or -(A¹O(O)C-A²-OC(O))ₐ—, where A¹ and A² can be, independently, an optionally substituted alkyl, cycloalkyl, aryl, or the like and “a” is an integer from 1 to 500.

The term “ether” as used herein is represented by a formula A¹OA², where A¹ and A² can be, independently, an optionally substituted alkyl, cycloalkyl, aryl, or the like. The term is inclusive of “polyether,” which, as used herein, is represented by a formula -(A¹O-A²O)ₐ—, where A¹ and A² can be, independently, an optionally substituted alkyl, cycloalkyl, aryl, or the like and “a” is an integer of from 1 to 500.

The term “thiol” as used herein is represented by a formula —SH.

In some embodiments the fluorophore can be an active agent, such as BODIPY® 650. The term “active agent” is used herein to refer to compounds or entities that alter, promote, speed, prolong, inhibit, activate, eliminate, or otherwise affect biological or chemical events in a subject. Still further, some embodiments of the compounds of the present disclosure can further comprise a second active agent, and in particular embodiments the second active agent comprises a second fluorophore.

Active agents of the present disclosure also include, but are not limited to, enzymes, organic catalysts, ribozymes, organometallics, proteins, glycoproteins, peptides, polyamino acids, antibodies, nucleic acids, steroidal molecules, antibiotics, antivirals, antimycotics, anticancer agents, analgesic agents, antirejection agents, immunosuppressants, cytokines, carbohydrates, oleophobics, lipids, extracellular matrix and/or its individual components, demineralized bone matrix, pharmaceuticals, chemotherapeutics, cells, viruses, virus vectors, and prions.

In some embodiments, the compounds can be tuned to be light-activated at a particular wavelength and/or over a given range of wavelengths. In some embodiments, the compounds can be tuned to be light-activated at certain wavelengths by appropriately selecting the fluorophore that is included in the compound.

In some embodiments, the compound comprises an active agent, and the compound can remain in an inert state until activated by light having a particular wavelength, thereby cleaving the active agent from the compound.

In some embodiments, the compounds can be tuned to be photo-activated by wavelengths that correspond to the wavelength of light absorbed by the fluorophore(s) appended to the compound. In some embodiments, the compounds are most rapidly activated via exposure to light having wavelengths that approximately correspond to the excitation spectrum of the appended fluorophore. In this regard, in some embodiments the compound is not photo-activated, or at least has a reduced rate of photo-activation when exposed to light having wavelengths that are shorter than those that excite the appended fluorophore.

In certain embodiments, the compound is not photo-activated, or at least has a reduced rate of photo-activation when exposed to light having wavelengths that are longer than those that excite the appended fluorophore. Furthermore, in some embodiments the compound is not photo-activated, or at least has a reduced rate of photo-activation, when exposed to light having wavelengths that are shorter than or longer than those that excite an appended fluorophore.

In this regard, the term “light” is used herein to refer to any electromagnetic radiation that can activate a compound. In some embodiments light includes ultraviolet light, visible light, near infrared light (NIR), or infrared light (IR). Compounds activated by relatively long wavelengths of light may be particularly well-suited for targeting tumors, and the like, and/or other targets that are deep in tissues, since light generally penetrates deeper into tissues as its wavelength increases. Some embodiments of compounds have the surprising and unexpected advantage of being photo-activated by light having wavelengths greater than 500 nm. Other embodiments of the compounds of the present disclosure can be photo-activated by light having wavelengths greater than 1000 nm.

More specifically, as used herein, light can refer to energy having a wavelength of about 500 nm to about 1300 nm. In specific embodiments, light can refer to energy having a wavelength of about 500 nm, about 550 nm, about 600 nm, about 650 nm, about 700 nm, about 750 nm, about 800 nm, about 850 nm, about 900 nm, about 950 nm, about 1000 nm, about 1050 nm, about 1100 nm, about 1150 nm, about 1200 nm, about 1250 nm, or about 1300 nm. In other specific embodiments, light can refer to energy having a wavelength greater than about 500 nm, greater than about 550 nm, greater than about 600 nm, greater than about 650 nm, greater than about 700 nm, greater than about 750 nm, greater than about 800 nm, greater than about 850 nm, greater than about 900 nm, greater than about 950 nm, greater than about 1000 nm, greater than about 1050 nm, greater than about 1100 nm, greater than about 1150 nm, greater than about 1200 nm, greater than about 1250 nm, and/or an even longer wavelength.

The presently-disclosed subject matter further includes pharmaceutical compositions comprising compounds as disclosed herein. Such pharmaceutical compositions may comprise at least one pharmaceutically-acceptable carrier. In this regard, the term “pharmaceutically acceptable carrier” refers to sterile aqueous or non-aqueous solutions, dispersions, suspensions or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions, which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

Suitable formulations include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets can be coated by methods known in the art.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in conventional manner.

The compounds can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt).

The compounds can also be formulated in rectal compositions (e.g., suppositories or retention enemas containing conventional suppository bases such as cocoa butter or other glycerides), creams or lotions, or transdermal patches.

The presently-disclosed subject matter further includes a kit that can include a compound or pharmaceutical composition as described herein, packaged together with a device useful for administration of the compound or composition. As will be recognized by those or ordinary skill in the art, the appropriate administration-aiding device will depend on the formulation of the compound or composition that is selected and/or the desired administration site. For example, if the formulation of the compound or composition is appropriate for injection in a subject, the device could be a syringe. For another example, if the desired administration site is cell culture media, the device could be a sterile pipette.

Still further, the presently-disclosed subject matter includes a method for treating disease(s), such as cancer. In some embodiments, the method comprises administering a compound, including one of the compounds described herein, to an administration site of a subject in need thereof, and then exposing the administration site of the subject to light after the compound has been administered. As described above, the light in some embodiments can be a light having a wavelength of about 500 nm to about 1300 nm. In this regard, longer wavelength light can be particularly useful for targeting deep tissue.

In some methods of the present disclosure, a plurality of compounds is administered to a subject, and the administration site(s) is then exposed to light having different wavelengths in a predetermined sequence. Accordingly, in such embodiments, the administration site can be sequentially subjected to effects of different active agents in a predetermined sequence without having to administer compounds at multiple time points. Thus, a subject can be treated by different active agents merely by adjusting the wavelength of the light that the administration site is exposed to.

Still further, in some methods, the compounds, after being administered, are internalized via the endosomal pathway of a subject's cells. Subsequently, when the cells are exposed to light, the active agent can be cleaved from the compound and/or released from endosomes into the cytosol. Through this process, some embodiments are capable of not damaging cells until the cells are exposed to light having a wavelength that activates the compound.

The term “administering” refers to any method of providing a compound and/or pharmaceutical composition thereof to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition (e.g., cancer, tumors, etc.). In further various aspects, a preparation can be administered prophylactically; that is, administered for prevention of a disease or condition.

In some embodiments, a subject will be administered an effective amount of the compound. In this respect, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

Additionally, the terms “subject” or “subject in need thereof” refer to a target of administration, which optionally displays symptoms related to a particular disease, pathological condition, disorder, or the like. The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “subject” includes human and veterinary subjects.

In some embodiments, the subject will be suffering or will have been diagnosed with one or more neoplastic or hyperproliferative diseases, disorders, pathologies, or conditions. Thus, an administration site to be exposed in a subject may be in close proximity or at the location of such a disease, condition, etc. (e.g., tumor). Examples of such diseases, conditions, and the like include, but are not limited to, neoplasms (cancers or tumors) located in the colon, abdomen, bone, breast, digestive system, esophagus, liver, pancreas, peritoneum, endocrine glands (adrenal, parathyroid, pituitary, testicles, ovaries, cervix, thymus, thyroid), eye, head and neck, nervous (central and peripheral), lymphatic system, pelvis, skin, soft tissue, spleen, thoracic areas, bladder, and urogenital system. Other cancers include follicular lymphomas, carcinomas with p53 mutations, and hormone-dependent tumors, including, but not limited to colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostate cancer, Kaposi's sarcoma and ovarian cancer, or metastases thereof.

A subject may also be in need because (s)he has acquired diseases or conditions associated with abnormal and increased cell survival such as, but not limited to, progression and/or metastases of malignancies and related disorders such as leukemia (including acute leukemias (e.g., acute lymphocytic leukemia, acute myelocytic leukemia, including myeloblastic, promyelocytic, myelomonocytic, monocytic, and erythroleukemia) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma. The conditions, diseases, and the like described above, as well as those that will be apparent to those of ordinary skill in the art, are collectively referred to as “cancer” herein.

The terms “treatment” or “treating” refer to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

With regard to the step of exposing an administration site to light, the method of exposing can be modified to meet the needs of a particular situation. Accordingly, the light can comprise sunlight, photo-optic light, and/or laser light. Further, in some embodiments, the light comprises ultraviolet light, visible light, near infrared light, or infrared light. Moreover, the light can be exposed from a laser light source, a tungsten light source, a photooptic light source, and the like. Light can also be provided at relatively specific administration site, and can be provided, for example, by the use of laser technology, fibers, endoscopes, biopsy needles, probes, tubes, and the like. Such probes, fibers, or tubes can be directly inserted, for example, into a body cavity or opening of a subject or under or through the skin of a subject, to expose the compound(s) that has been administered to the subject to light.

Light sources can also include dye lasers or diode lasers. Diode lasers may be advantageous in certain applications due to their relatively small and cost-effective design, ease of installation, automated dosimetry and calibration features, and longer operational life. Certain lasers, including diode lasers, also operate at relatively cool temperatures, thereby eliminating the need to supply additional cooling equipment. In some embodiments, the light source is battery-powered. Also, the light source can be provided with a diffuse tip or the like, such as an inflatable balloon having a scatting material.

Light can be provided to a subject at any intensity and duration that provides the required photo-activation for a particular application. In some embodiments, the methods of treatment provided in the present disclosure comprise administering relatively low doses of the compound and/or exposing an administration site to relatively low intensity light over the course of several hours or days. In some embodiments, this low-dose technique can allow for excellent tumor control while minimizing normal tissue damage.

Rheumatoid arthritis (RA) is a progressive inflammatory autoimmune disease that afflicts just under 1% of the United States population. (Majithia, 2007). RA is responsible for a quarter of a million hospitalizations and 10 million physician visits per year. A 2010 report put RA's societal costs at $40 billion (in 2005 dollars). (Birnbaum, 2010) RA patients typically present symptoms that include joint pain, swelling, tenderness, and warmth. Although the underlying cause remains to be ascertained, the polyarticular symptoms are a consequence of the influx of lymphocytes and monocytes into the synovium/joint space, the production of pro-inflammatory cytokines, bone and cartilage destruction, and disease spread to other joints and subsequent systemic effects. Ultimately, this leads to irreversible joint damage, disfigurement, and disability. It has been known for more than half a century that the direct injection of anti-inflammatory agents into the afflicted joints provides therapeutic benefits. (Hollander, 2951). However, multiple injections into multiple joints on a routine basis is not a viable therapeutic option. (Mitragotri, 2011)

The recent introduction of “biologics” (e.g. antibodies that target TNFα, IL-1, etc.), along with aggressive treatment using other drugs at the time of diagnosis, has reduced the rate of progression of RA. (Kukar, 2009) Nonetheless, RA therapies generally require frequent and long-term drug administration, which commonly results in undesired side effects ranging from moderate to severe. For example, the approximately 50% of RA patients who are dependent upon glucocorticoids (Huscher 2009) must deal with the consequences of their long-term use, which includes weight gain, osteoporosis, diabetes mellitus, hypertension, skin fragility and infections arising from being systemically immunocompromised. (Basschant, 2012). Not surprisingly, there is significant interest in the development of therapeutics that can be selectively delivered to RA joints in order to reduce undesired systemic effects. (Mitragotri 2011; Fiehn 2010; Ulbrich 2010).

Since combination therapy is favored for RA patients, any delivery system must be robust enough to dispense different drugs with exquisite spatial and temporal control. One possibility, which has seen intense application in the management of cancer, is the use of light to activate therapeutic agents at disease sites. For example, photodynamic therapy delivers localized bursts of cytotoxic O₂ to those tissues that are marked for destruction. (Shirasu 2013) More recently, the development of light-activated pro-drugs has received attention. (Shamay 2011; Thompson 2010; Yavlovich 2010). However, in general, the latter are limited by the fact that short wavelengths (<450 nm), with poor tissue penetrating power, are required for photo-activation. Furthermore, within this narrow wavelength range, the ability to discriminate between different pro-drugs in a wavelength-specific fashion is very limited.

In some embodiments of the presently-disclosed subject matter, light responsive constructs function within the optical window of tissue (600-1000 nm). In some embodiments, light-responsive constructs are encoded to respond in a wavelength-specific fashion, resulting in triggering different biological actions (e.g. release of different drugs). In some embodiments, the compounds are used to treat diseases, including but not limited to rheumatoid arthritis, cancer, and diabetes.

In some embodiments of the present disclosure, a drug delivery system using red blood cells (RBCs) is disclosed.

Erythrocytes have been described as the “champions [of] drug delivery systems”. (Muzykantov 2010) They are biocompatible, have a life span of up to 120 days, and are of a size that vastly exceeds those of other drug carriers so that relatively large drug quantities can be conveyed. However, “practically useful controlled release from carrier RBC (red blood cells) remains an elusive goal.” (Muzykantov 2010) Light-controlled release using conventional photo-labile reagents is not feasible due to the presence of hemoglobin, which consumes the short wavelength region of the visible spectrum (up to 600 nm).

In some embodiments, the present disclosure provides an RBC-based drug delivery system that overcomes this limitation and offers, for the first time, controlled release of therapeutic agents from RBCs in a spatially and temporally controlled fashion.

A new family of peptide-based RA therapeutics is receiving significant attention due to promising immuno-modulating, but not immuno-suppressing, properties. (Getting 2009; Luger 2007; Yang 2013) However, these peptides are rapidly degraded in the blood. In some embodiments, the presently-disclosed subject matter provides systems and methods to deliver peptides to treat diseases in a subject in need thereof. Particularly, some embodiments of the present disclosure provide drug delivery systems and methods for stabilizing peptides in a protective sheath and delivering the peptides to their intended site of action, where, subsequently, the peptides are locally released when exposed to light.

It has been argued that “successful new therapies [for RA] need not only be efficacious with a good or improved safety profile but must also be formulated in such a way as to allow self-administration by patients”. (Minter 2013) In some embodiments of the presently-disclosed subject matter, a therapeutic method is provided that, in conjunction with existing light-delivery systems (e.g. the technology used in “low level laser therapy” (Bjordal 2008)), places therapeutic application at the site-of-inflammation in the hands of the patient.

Many light-activatable versions of biologically active reagents, including small molecules, peptides, proteins, and nucleic acids have been reported. (Lee 2009) Accordingly, the strategy requires (a) identification of a key functional group on the agent that is essential for biological activity and (b) its covalent modification with a light-cleavable moiety. This strategy is often traced back to a seminal 1978 paper that described a light-activatable ATP. (Kaplan 1978) The latter is not recognized by an ATPase. However, upon photolysis (˜330 nm), ATP is generated and subsequently hydrolyzed. Two criteria dictate the photo-cleavage wavelength: (a) the spectrum of the resident chromophore (e.g. the nitrobenzyl group) and (b) the nature of the bond to be cleaved (e.g. the nitrobenzyl C—O). For any photosensitive group, there is a minimum energy required to effect efficient photo-cleavage. (Aujard 2006) A variety of other photo-cleavable/photo-convertible moieties have been described (Klan 2013) and though their absorbance wavelengths vary to some extent (350-500 nm), their photolytic wavelength-dependency is pre-determined by the two criteria enumerated above.

In some embodiments of the present disclosure, a new strategy is provided for the creation of photo-activatable agents. This strategy represents a marked departure from the approach that has been in use since 1978. In some embodiments, the present disclosure provides that (a) wavelengths with maximal tissue penetration (for example, 600-900 nm) are used for drug activation, (b) specific wavelengths can be encoded for different light-activatable therapeutics, thereby enabling wavelength-dependent discrimination, and (c) the photo-responsive constructs can be attached to any position on the drug/agent-of-interest, thereby eliminating the constraint that a key functionality essential for bio-molecule activity must be covalently modified with a photo-cleavable group.

Although peptides continue to receive a great deal of attention for their therapeutic potential, most are rapidly cleared and/or degraded in the blood. The present disclosure provides in some embodiments a roteolytically susceptible peptide that can be “hidden” in the protein sheath that enshrouds the plasma membrane of RBCs is demonstrated. The latter will be coupled with wavelength-encoded constructs, thereby promoting peptide release at the desired biological site and thus limiting exposure to proteases.

In some embodiments, an engineered, three-dimensional model of the arthritic arterial synovial joint interface (containing multiple human cell lines) is used to assess the efficacy of the wavelength-encoded drug delivery technology.

### EXAMPLES

The presently-disclosed subject matter is further illustrated by the following specific but non-limiting examples. The examples may include compilations of data that are representative of data gathered at various times during the course of development and experimentation related to the presently-disclosed subject matter.

The following examples describe, among other things, the synthesis and characterization of certain exemplary alkylcobalamin compounds that are appended with fluorphores.

Hydroxocobalamin hydrochloride (B₁₂ₐ) was purchased from MP Biomedicals. TAMRA was purchased from AnaSpec. SulfoCy5 succinimidyl ester was purchased from Lumiprobe. BODIPY® 650 succinimidyl ester, MitoTracker® Green, and Rhodamine B dextran (10 000 MW) were purchased from Invitrogen. Dylight® 800 succinimidyl ester was purchased from Thermo Fisher Scientific. All other fluorophores and reagents were purchased from Sigma-Aldrich. All fluorophores and reagents were used without further purification. The 546±10 nm bandpass filter was purchased from Newport. The 646±10, 700±10, 730±10, and 780±10 nm bandpass filters were purchased from Cheshire Optical. All imaging was performed on an Olympus IX81 inverted fluorescence microscope with a Lambda LS3 xenon arc lamp and a Hamamatsu C8484 CCD camera.

β-(3-aminopropyl)cobalamin 1 was prepared from hydroxocobalamin and 3-chloropropylamine hydrochloride according to a literature procedure. (Smeltzer, C. C.; Cannon, M. J.; Pinson, P. R.; Munger, J. D., Jr.; West, F. G.; Grissom, C. B Org. Lett., 2001, 3, 799-801.) Purification was achieved according to literature procedure to afford an orange solid (0.0154 g, 68%); ESI MS calculated for C₆₅H₉₈N₁₄O₁₄PCo (M¹⁺): m/z=1388.7. found 1388.7; calc. for (M²⁺) m/z=694.3. found 694.5; calculated for (M³⁺): m/z=463.1. found 462.9. (Priestman, M. A.; Shell, T. A.; Sun, L.; Lee, H.-M.; Lawrence, D. S. Angew. Chem. 2012, 124, 7804-7807; Angew. Chem. Int. Ed. 2012, 51, 7684-7687.)

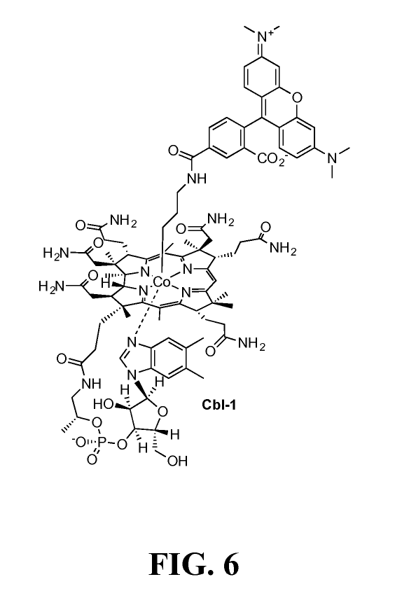

Synthesis of Cobalamin-TAMRA Conjugate (Cbl-1) was conducted as shown in FIG. 6: Cbl-1 was prepared from β-(3-aminopropyl)cobalamin 1 and 5-carboxytetramethylrhodamine (TAMRA) according to a literature procedure. Purification was achieved according to a literature procedure to afford a red solid, 82%; ESI MS calculated for C₉₀H₁₁₈N₁₆O₁₈PCo (M²⁺): m/z=900.4. found 900.5; calculated for (M³⁺): m/z=600.3. found 600.3. (Priestman, M. A.; Shell, T. A.; Sun, L.; Lee, H.-M.; Lawrence, D. S. Angew. Chem. 2012, 124, 7804-7807; Angew. Chem. Int. Ed. 2012, 51, 7684-7687.)

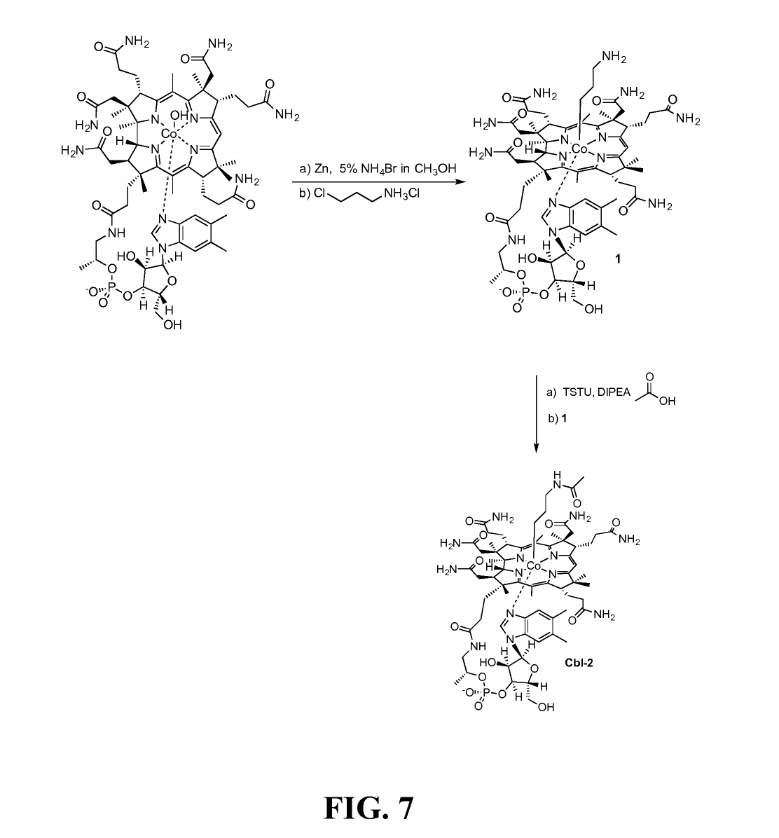

Synthesis of β-(3-acetamidopropyl)cobalamin (Cbl-2) was achieved as shown in FIG. 7: N,N,N′,N′-Tetramethyl-O—(N-succinimidyl)uranium tetrafluoroborate (TSTU, 0.0242 g, 80 μmol), acetic acid (0.0022 g, 38 μmol) and DIPEA (0.0234 g, 181 μmol), were mixed for 1 h in a 2:2:1 dimethylformamide:dioxane:water solution (250 μL). β-(3-aminopropyl)cobalamin 1 (0.0052 g, 3.7 μmol) was added and the reaction, and the reaction was mixed for 18 hours. The desired compound was purified by HPLC (semiprepative C-18 column) using a linear gradient binary solvent system (solvent A: 0.1% TFA/H₂O; solvent B: 0.1% TFA/CH₃CN) with a ratio of A:B that varied from 97:3 (0 min) to 10:90 (40 min). Removal of solvent by lyophilization afforded an orange solid (0.0039 g, 73%); ESI MS calculated For C₆₇H₁₀₀N₁₄O₁₅PCo (M⁺): m/z=1430.7. found 1431.7; calculated for (M²⁺): m/z=715.3. found 715.5.

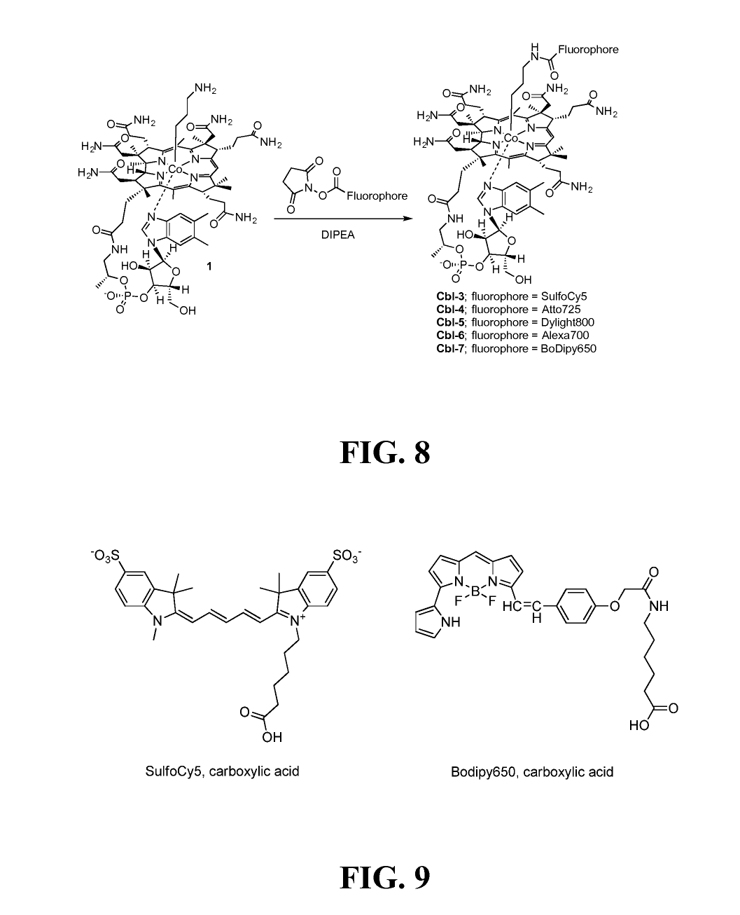

Synthesis and characterization of cobalamin-fluorophore conjugates (Cbl-3, Cbl-4, Cbl-5, Cbl-6, and Cbl-7) was achieved as shown in FIG. 8.

General synthesis of cobalamin-fluorophore conjugates: The N-hydroxysuccinimide ester of a fluorophore (1 eq.), β-(3-aminopropyl)cobalamin 1 (1.5 eq.), and diisopropylethylamine (6 eq.) were mixed in dimethylformamide for 18 hours. The desired compound was purified by HPLC (semiprepative C-18 column) using a linear gradient binary solvent system (solvent A: 0.1% TFA/H₂O; solvent B: 0.1% TFA/CH₃CN) with a ratio of A:B that varied from 97:3 (0 min) to 10:90 (40 min). Removal of solvent by lyophilization afforded a solid.

Cobalamin-SulfoCy5 Conjugate (Cbl-3) is shown in FIG. 9: blue solid, 89%; ESI MS calculated for C₉₇H₁₃₄N₁₆O₂₂PS₂Co (M²⁺): m/z=1014.4. found 1013.6; calculated for (M³⁺): m/z=676.3. found 676.4.

Cobalamin-ATTO 725 Conjugate (Cbl-4): blue solid, 66%; ESI MS estimated for C₉₀H₁₁₈N₁₆O₁₈PCo-ATTO 725 (M²⁺): m/z=892.9. found 893.2; estimated for (M³⁺): m/z=595.3. found 595.9. (The formula and exact mass of ATTO 725, carboxylic acid have not been reported.)

Cobalamin-Dylight® 800 Conjugate (Cbl-5): blue solid, 92%; ESI MS estimated for C₉₀H₁₁₈N₁₆O₁₈PCo-Dylight800 (M²⁺): m/z=1141.1. found 1139.8; estimated⁴ for (M³⁺): m/z=760.7. found 760.4. (The formula and exact mass of Dylight® 800, carboxylic acid have not been reported.)

Cobalamin-Alexa Fluor® 700 Conjugate (Cbl-6): blue solid, 72%, ESI MS found for C₉₀H₁₁₈N₁₆O₁₈PCo-Alexa Fluor® 700 (M²⁺): m/z=1179.9. found. for (M³⁺): m/z=787.0. (The formula and an accurate mass of Alexa Fluor® 700, carboxylic acid have not been reported.)

Cobalamin-BODIPY® 650 Conjugate (Cbl-7) as shown in FIG. 9: blue solid, 88%, ESI MS calculated for C₉₄H₁₂₄N₁₈O₁₇PBF₂Co (M²⁺): m/z=957.9. found 958.7; calculated for (M³⁺): m/z=638.3. found 638.6.

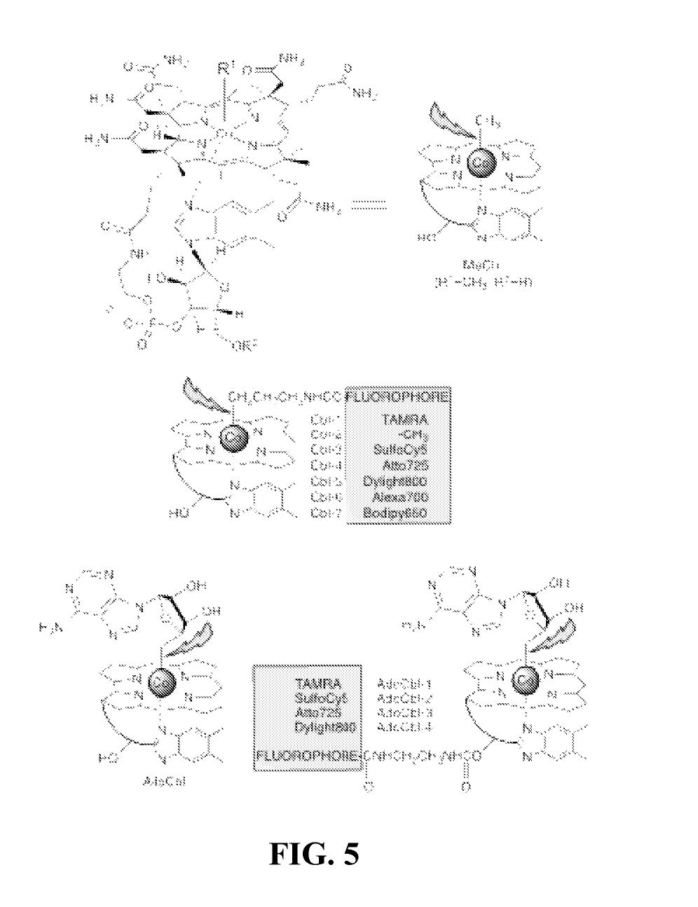

Cbl-1, which contains an alkyl-tetramethyl-rhodamine (TAMRA) moiety was affixed to the cobalt center of an alkylcobalamin, (Priestman, M. A., et al., Angew. Chem. Int. Ed. Engl. 2012) and served as a reporter of photocleavage. FIG. 5 is includes structures of alkyl-cobalamins and alkyl-cobalamin-fluorophore conjugates. FIG. 6 is Scheme 51, which is the structure of cobalamin-TAMRA conjugate (Cbl-1). A fluorescence increase was observed upon photolysis of Cbl-1 due to the ability of cobalamin to quench the fluorescence of attached fluorophores. (Priestman, M. A., et al., Angew. Chem. Int. Ed. Engl. 2012. Lee, M.; et al., Org. Lett. 2009. Smeltzer, C. C., et al., Org. Lett. 2001. Jacobsen, D. W. Methods Enzym. 1980. Rosendahl, M. S.; et al., Proc. Nat. Acad. Sci. USA 1982. Jacobsen, D. W., et al., J. Inorg. Biochem. 1979.) The latter was a consequence of contact quenching between the fluorophore and the corrin ring system. (Lee, M.; Grissom, C. B. Org. Lett. 2009) Without being bound by theory or mechanism, contact quenching, unlike fluorescence energy resonance transfer, does not require an overlap between the emission and absorption wavelengths of the fluorophore and quencher, respectively, for energy transfer to transpire. Given the fact that the Co-alkyl bond is weak, (<30 kcal/mol), (Halpern, J., et al., J. Am. Chem. Soc. 1984. Kozlowski, P. M., et al., J. Chem. Theory Comput. 2012 and references cited therein.) it appeared that fluorophores excited at wavelengths beyond that absorbed by cobalamin (>560 nm) could transfer their excited state energy to the corrin ring and thus promote Co—C bond scission.

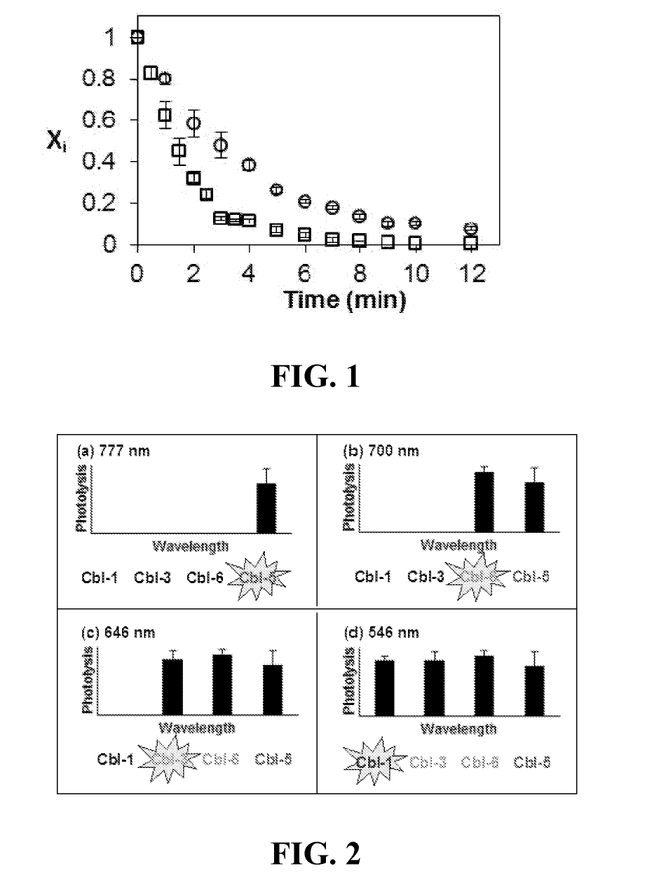

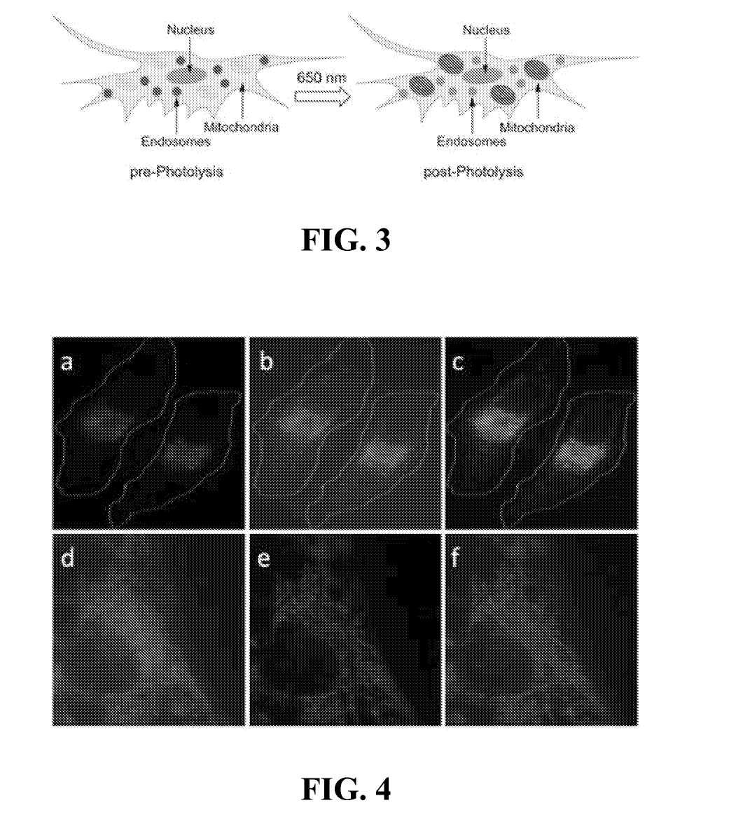

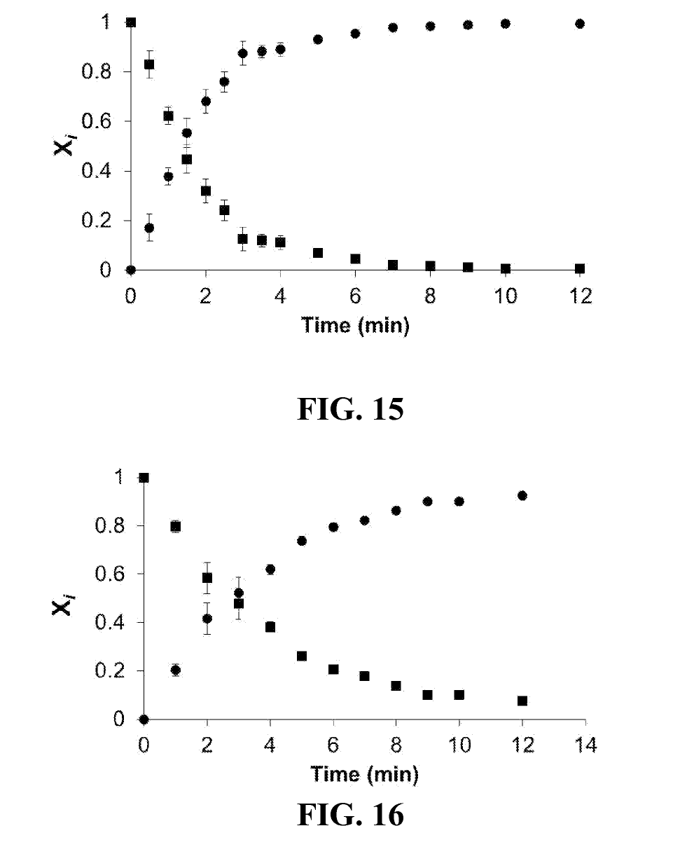

The TAMRA and corrin moieties in Cbl-1 absorb 500-570 nm light. A TAMRA-mediated energy transfer mechanism could accelerate Co—C bond cleavage relative to that of methylcobalamin (MeCbl) and a model alkylcobalamin that does not have an appended fluorophore, Cbl-2. Both MeCbl and Cbl-2 (1.9±0.2 and 2.1±0.3 μM-1/min, respectively, FIGS. 14 and 15) were photolyzed at similar rates. By contrast, Cbl-1 suffered photolysis at twice the rate (3.8±0.3 μM-1/min) of its non-fluorophore-containing counterparts, MeCbl and Cbl-2 (FIGS. 1 and 16). Therefore, an appended fluorophore can play a role in promoting photocleavage of the Co—C bond.

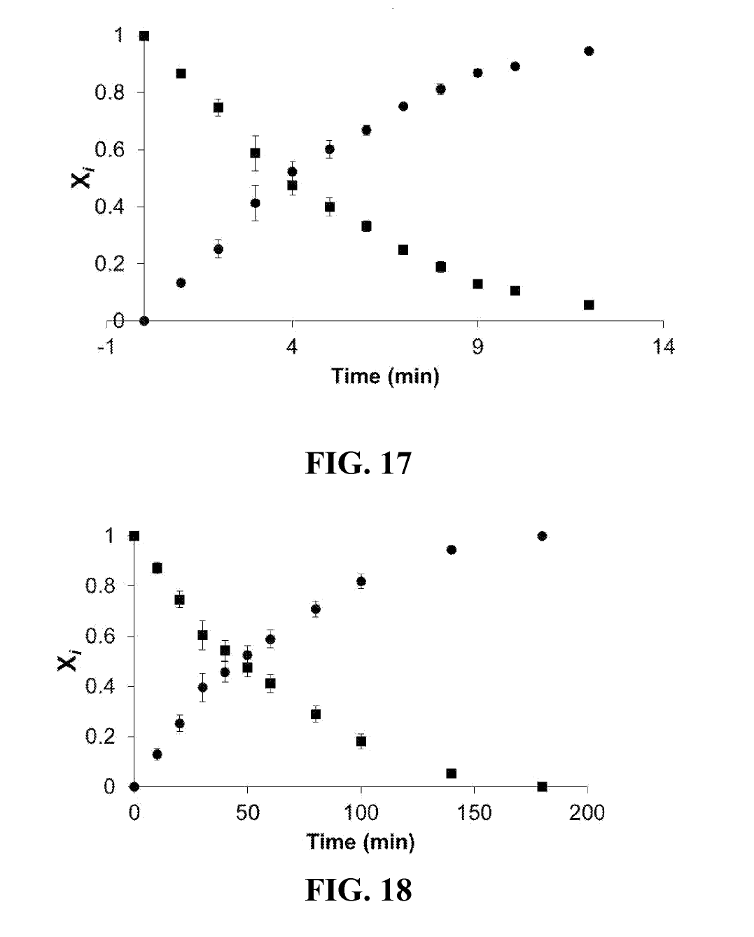

Several fluorophores were appended, all with excitation wavelengths longer than that absorbed by the corrin ring, to the alkylcobalamin framework. The SulfoCy5 derivative Cbl-3 (λₑₓ 650 nm, λₑₘ 660 nm) was photolyzed (646±10 nm) and completely converted to B₁₂ₐ as assessed by UV-Vis (FIG. 17) and LC/MS (Table 3). The latter revealed the formation of three SulfoCy5 derivatives, an aldehyde, a hydroperoxide that is converted into the aldehyde, and an alkyl product (Scheme S4). By contrast, Cbl-3 maintained in the dark, as well as Cbl-1 exposed to 646 nm (Table 1), retained their structural integrity.

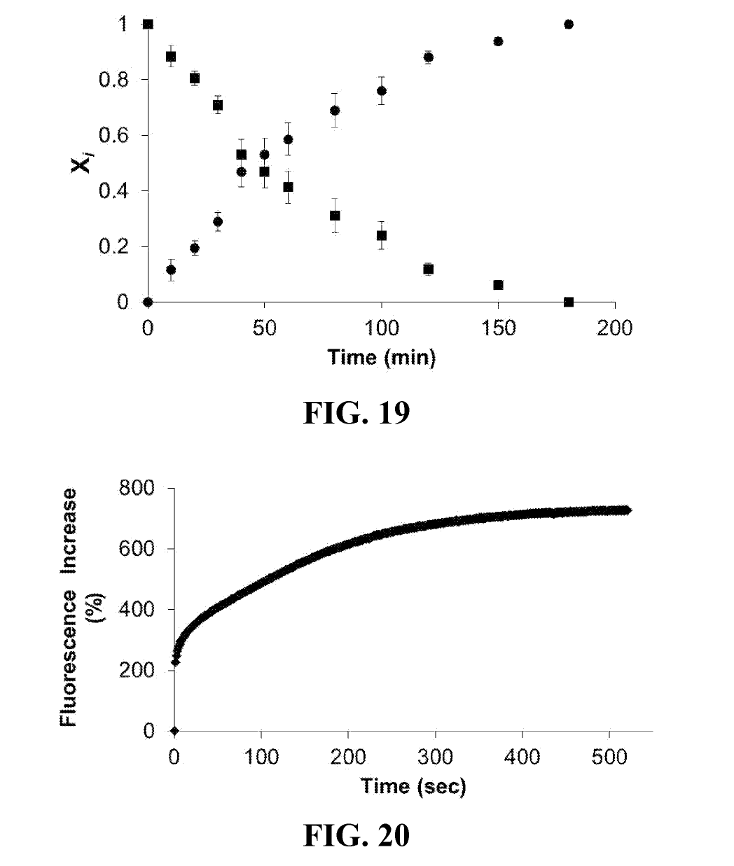

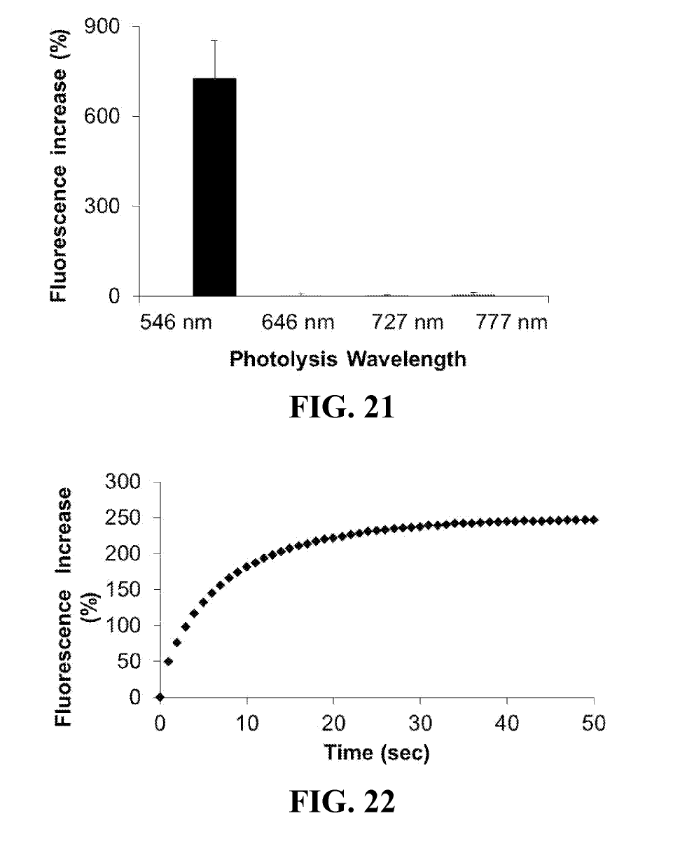

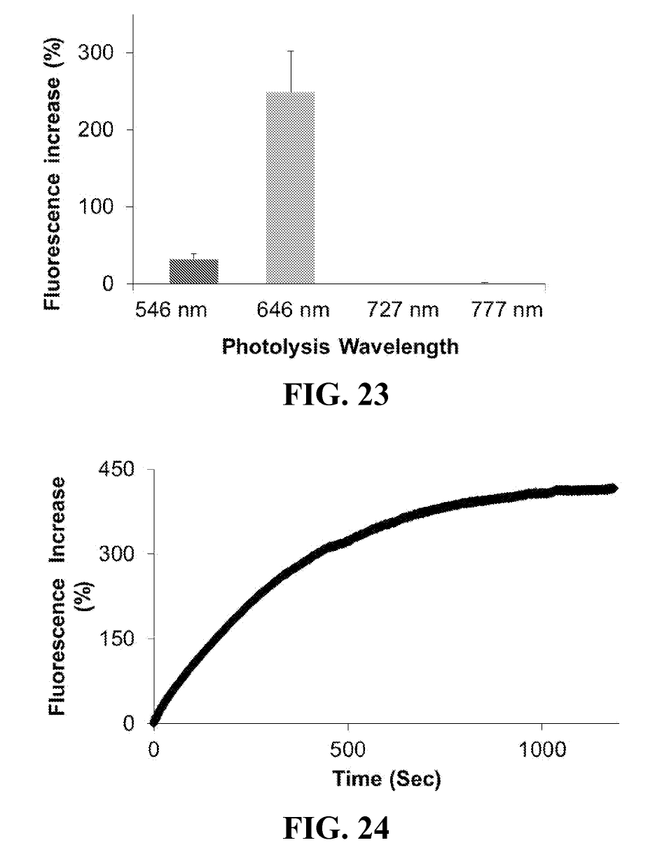

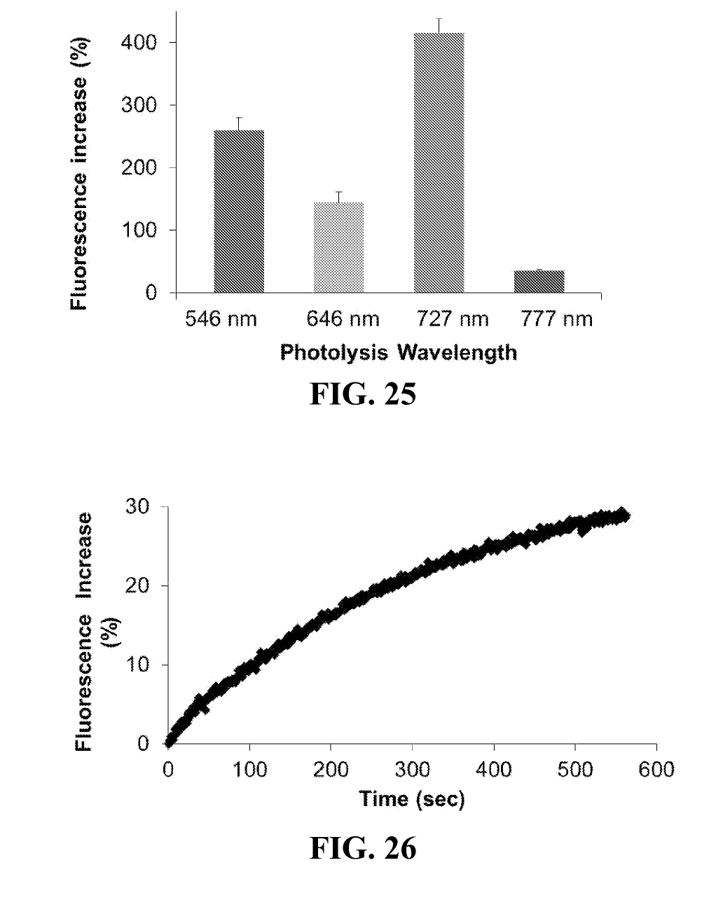

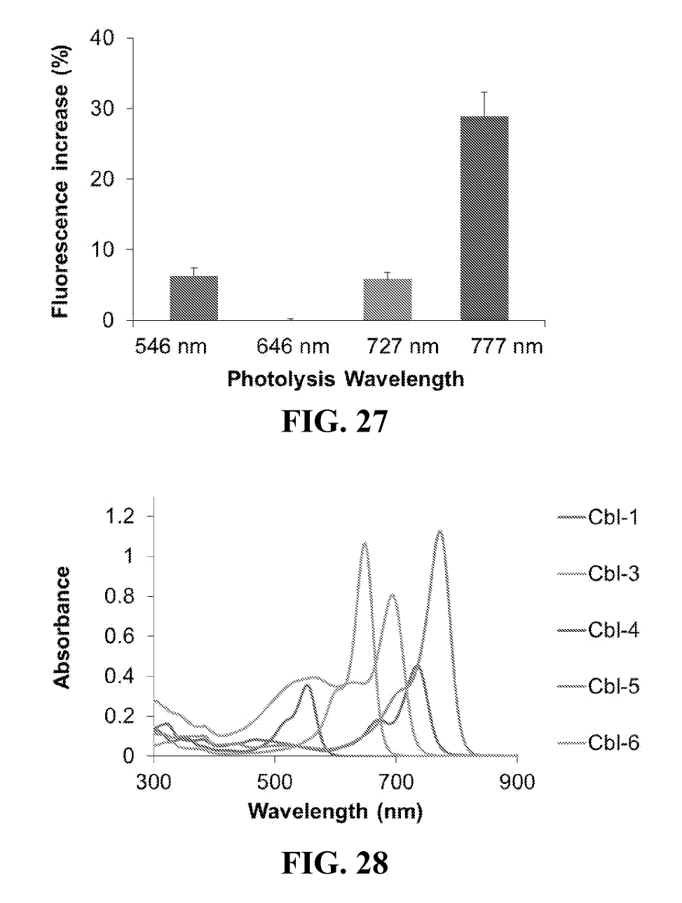

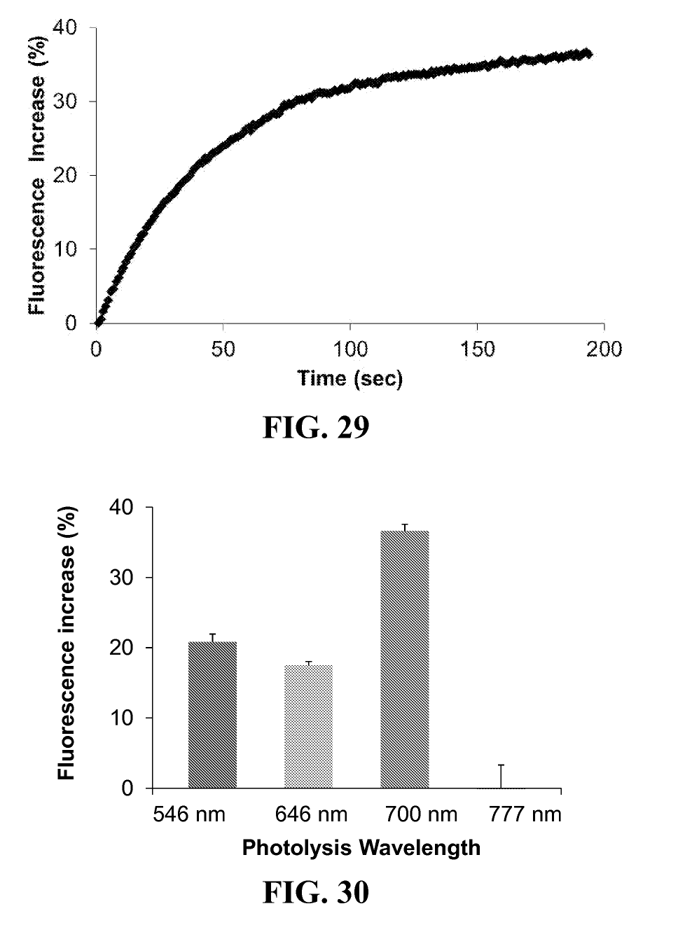

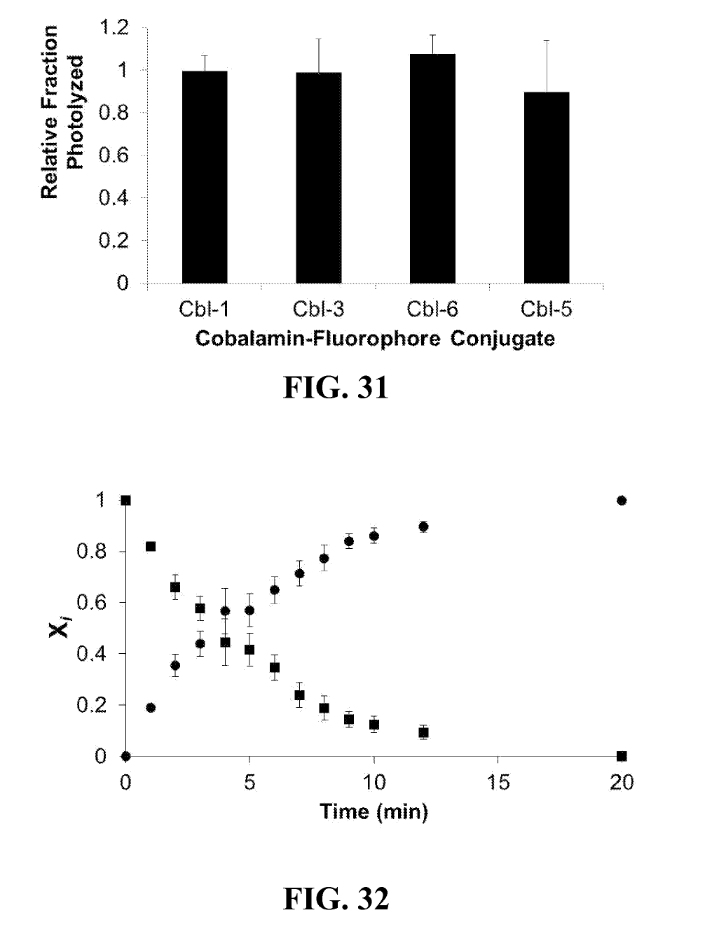

Like Cbl-1, Cbl-3 displayed a fluorescence enhancement upon photolysis (Table 1 and FIG. 21). The Atto725 (Cbl-4, λₑₓ 730 nm, λₑₘ 750 nm) and Dylight® 800 (Cbl-5, λₑₓ 775 nm, λₑₘ 794 nm) derivatives were also prepared and examined. Photolysis of Cbl-4 and Cbl-5 at 730±10 and 780±10 nm, respectively, was validated by LC/MS (FIGS. 18 and 19, Tables 4 and 5). As with Cbl-3, both Cbl-4 and Cbl-5 were stable in the dark as confirmed by LC/MS. In addition, exposure of the TAMRA derivative, Cbl-1, to these long wavelengths had no observable effect on its structural integrity (Table 1).

To assess the potential for wavelength-selective photocleavage, each cobalamin-fluorophore conjugate was exposed to 546, 646, 727, and 777 nm, as outlined in Table 1. Cbl-1 does not absorb light beyond 600 nm, and it was unaffected by exposure to >600 nm light. In an analogous fashion, Cbl-3, Cbl-4, and Cbl-5 showed little or no effect upon exposure to 546 nm (Table 1) or to wavelengths longer than those absorbed by these compounds. For example, Cbl-3, with an appended fluorophore that absorbs 650 nm light, was inert to 727 and 777 nm irradiation. The ATTO 725-appended Cbl-4 had a significant shoulder absorption at 646 nm and a very weak absorption at 777 nm. Cbl-4 responded in a predictable fashion, by displaying modest photolysis at 646 nm and very minor photolysis at 777 nm. Finally, the Dylight® 800-modified Cbl-5 was impervious to photolysis at 646 nm and 727 nm but, as expected, responded to 777 nm. Without being bound by theory or mechanism, these tested exemplary embodiments demonstrate that cobalamin-fluorphore conjugates can photolyze rapidly in response to wavelengths that match the excitation spectrum of the appended fluorophore.

TABLE 1
Percent fluorescence increase of cobalamin-fluorophore conjugates
upon photolysis at 546 nm (5 min), 646 nm (5 min), 727 nm (20 min)
and 777 nm (10 min) light, where * <6%.

% Fluorescence Increase @ λₑₓ

| | 546 nm | 646 nm | 727 nm | 777 nm |
|---|---|---|---|---|
| Cbl-1 | 730 ± 130 | * | * | * |
| Cbl-3 | 32 ± 7 | 250 ± 50 | * | * |
| Cbl-4 | 260 ± 20 | 140 ± 20 | 420 ± 20 | 36 ± 2 |
| Cbl-5 | * | * | * | 29 ± 3 |
| Cbl-6 | 21 ± 5 | 17 ± 3 | 37 ± 1 | * |
| Cbl-7 | 110 ± 10 | 220 ± 30 | * | * |

The results in Table 1 suggest that it is possible to selectively photolyze specific compounds in a mixture of cobalamin-substituted derivatives by employing the appropriate illumination sequence and choice of wavelengths. Due to the slight amount of photolysis displayed by Cbl-4 at 777 nm, the latter was replaced with the Alexa Fluor® 700-appended conjugate Cbl-6 (700 nm sensitive, but 777 nm resistant). Illumination of a mixture of Cbl-5, Cbl-6, Cbl-3, Cbl-1 at 777 nm, furnished only Cbl-5 photolysis (FIG. 2a). Subsequent exposure of the mixture to 700 nm elicited the conversion of Cbl-6 to its photoproducts without affecting Cbl-3 or Cbl-1 (FIG. 2b). Finally, sequential illumination at 646 and 546 nm (FIGS. 2c and 2d), selectively photolyzed Cbl-3 and Cbl-1, respectively. Therefore, not only was it possible to tune photo-release to specific wavelengths but, by the proper choice of fluorophores and the illumination wavelength sequence, four compounds were orthogonally and sequentially photolyzed in a predictable fashion.

Further, the following examples examine the synthesis and characteristics of a compound wherein of a fluorophore appended to the ribose 5′-OH of an alkylcobalamin. Specifically, the following examples examine whether such compounds can be used to promote photocleavage of the Co-alkyl linkage (Chart 1, AdoCbl-1-AdoCbl-4).

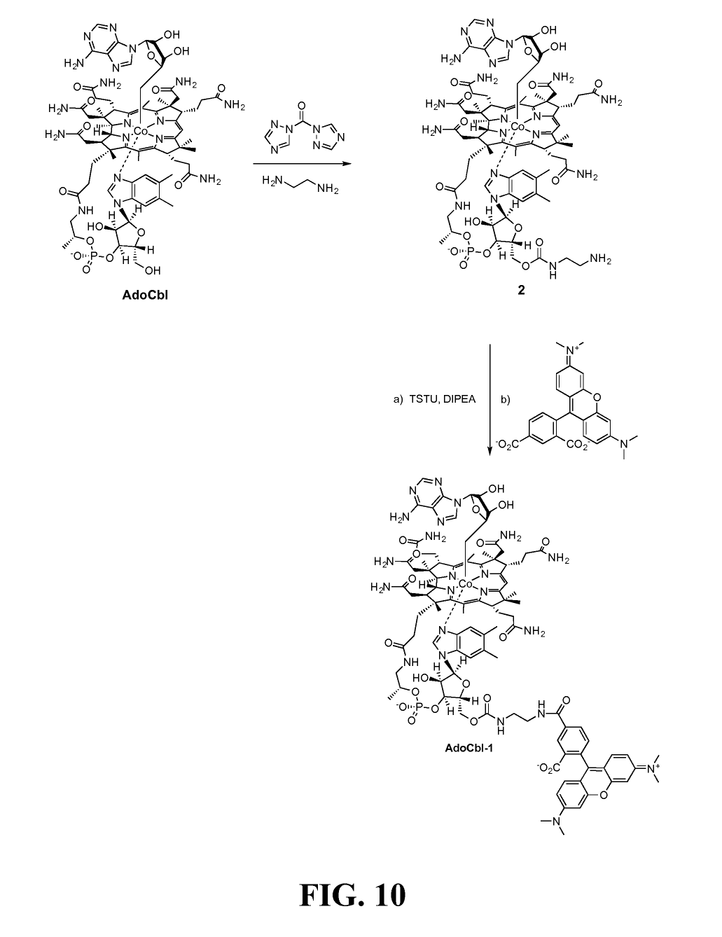

Synthesis and characterization of coenzyme B₁₂-TAMRA conjugate (AdoCbl-1) is shown in FIG. 10.

### Synthesis of Coenzyme B₁₂-Ethylenediamine Conjugate 2:

Coenzyme B₁₂ (0.0209 g, 13 μmol) and 1,1-carbonyldi-(1,2,4-triazole) (0.0142 g, 87 μmol) were added to an oven-dried round bottom flask. The vessel was purged with Ar. Dry dimethylformamide (0.2 mL) was added to the flask and the mixture was stirred at room temperature for 1 h. Ethylenediamine (0.0270 g, 450 μmol) was added to the reaction mixture and stirring continued for another 18 h. The desired compound was purified by HPLC (semiprepative C-18 column) using a linear gradient binary solvent system (solvent A: 0.1% TFA/H₂O; solvent B: 0.1% TFA/CH₃CN) with a ratio of A:B that varied from 97:3 (0 min) to 10:90 (40 min). Removal of solvent by lyophilization afforded an orange solid (0.0189 g, 86%); ESI MS calculated for C₇₅H₁₀₇N₂₀O₁₈PCo (M²⁺): m/z=832.9. found 833.4; calculated for (M³⁺): m/z=555.2. found 556.2.

Synthesis of coenzyme B₁₂-TAMRA conjugate (Ado-Cbl-1): N,N,N′,N′-Tetramethyl-O—(N-succinimidyl)uranium tetrafluoroborate (TSTU, 0.0139 g, 46 μmol), TAMRA (0.0127 g, 30 μmol) and DIPEA (0.0230 g, 178 μmol), were mixed for 2 hours in a 2:2:1 dimethylformamide:dioxane:water solution (250 μL). Coenzyme B₁₂-ethylenediamine conjugate 2 (0.0039 g, 2.3 μmol) was added, and the reaction was mixed for 18 hours. The desired compound was purified by HPLC (semiprepative C-18 column) using a linear gradient binary solvent system (solvent A: 0.1% TFA/H₂O; solvent B: 0.1% TFA/CH₃CN) with a ratio of A:B that varied from 97:3 (0 min) to 10:90 (40 min). Removal of solvent by lyophilization afforded a red solid (0.0026 g, 53%); ESI MS calculated For C₁₀₀H₁₂₈CoN₂₂O₂₂P (M²⁺): m/z=1039.4. found 1039.8; calculated for (M³⁺): m/z=693.0. found 693.6; calculated for (M⁴⁺): m/z=519.7. found 520.5.

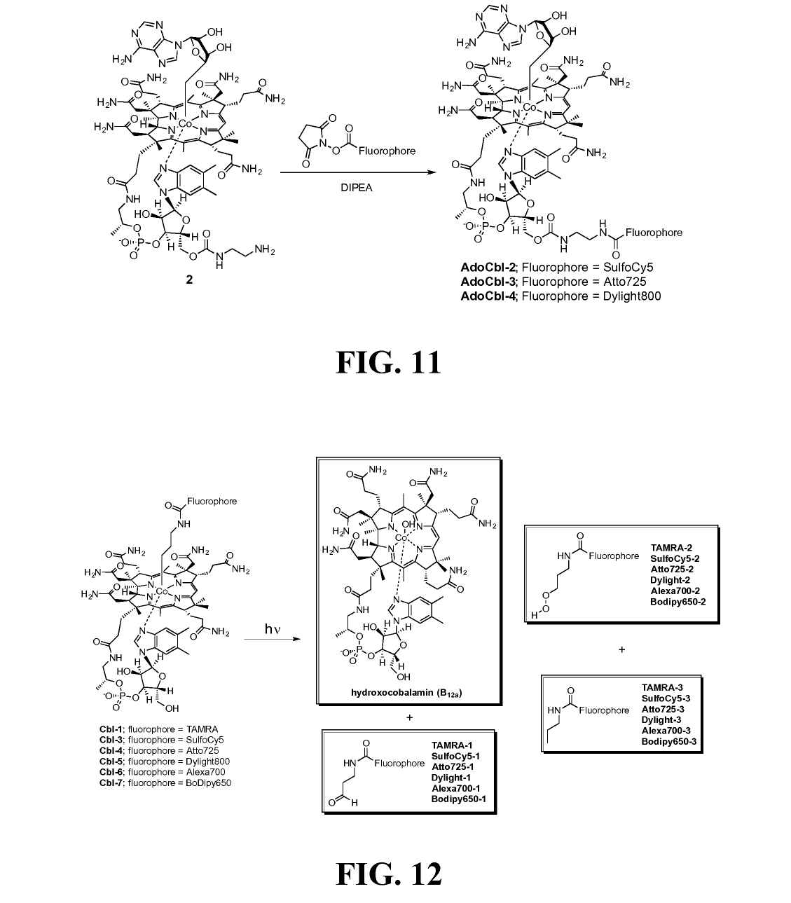

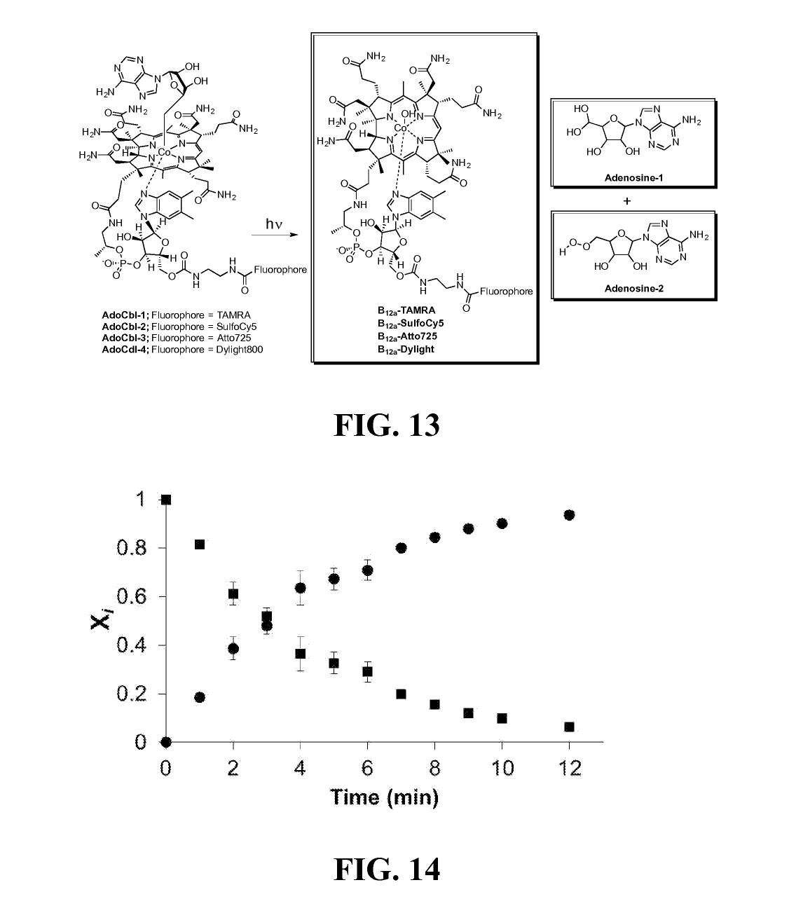

Synthesis and Characterization of coenzyme B₁₂-fluorophore conjugates (AdoCbl-2, AdoCbl-3, and AdoCbl-4) are shown in FIG. 11.

### General Synthesis of Cobalamin-Fluorophore Conjugates:

The N-hydroxysuccinimide ester of a fluorophore (1 eq.), coenzyme B₁₂-ethylenediamine conjugate 2 (1.5 eq.), and diisopropylethylamine (6 eq.) were mixed in dimethylformamide for 18 hours. The desired compound was purified by HPLC (semiprepative C-18 column) using a linear gradient binary solvent system (solvent A: 0.1% TFA/H₂O; solvent B: 0.1% TFA/CH₃CN) with a ratio of A:B that varied from 97:3 (0 min) to 10:90 (40 min). Removal of solvent by lyophilization afforded a solid.

Coenzyme B₁₂-SulfoCy5 Conjugate (AdoCbl-2): blue solid, 87%, ESI MS calculated for C₁₀₇H₁₄₂N₂₂O₂₆PS₂Co (M²⁺): m/z=1152.5. found 1152.7; calculated for (M³⁺): m/z=768.3. found 768.8.

Coenzyme B₁₂-ATTO 725 Conjugate: blue solid (AdoCbl-3), 69%, ESI MS estimated for C₇₅H₁₀₆N₂₀O₁₈PCo-Atto725 (M²⁺): m/z=1031.4. found 1032.3; estimated for (M³⁺): m/z=687.6. found 688.5. (The formula and exact mass of ATTO 725, carboxylic acid have not been reported.)

Coenzyme B₁₂-Dylight® 800 Conjugate (AdoCbl-4): blue solid, 90%, ESI MS estimated for C₇₅H₁₀₆N₂₀O₁₈PCo-Dylight® 800 (M²⁺): m/z=1279.6. found 1279.6; estimated for (M³⁺): m/z=853.1. found 853.2. (The formula and exact mass of Dylight® 800, carboxylic acid have not been reported.)

### Photolysis Rate Comparison of MeCbl, Cbl-1, Cbl-2, AdoCbl and AdoCbl-1

General procedure: Photolysis was performed using an Oriel Xe flash lamp (800 mJ, 62 Hz) with a selective bandpass filter for 546±10 nm. The conversion of alkylcobalamin to hydroxocoabalmin was determined by monitoring the absorption of the mixture at 350 nm using Perkin Elemer Lambda 2 UV/Vis spectrophotometer. (Taylor, R. T.; Smucker, L.; Hanna, M. L.; Gill, J. Arch. Biochem. Biophys. 1973, 156, 521-533.)

### Determination of Cbl-6 and Cbl-7 Photolysis Products

General procedure: Photolysis of Cbl-6 and Cbl-7 was performed using a Oriel Xe flash lamp (800 mJ, 62 Hz) with a selective bandpass filter for 700 and 646±10 nm, respectively, for 2 h. The resulting sample (100 μL) was analyzed by LC/MS using a linear gradient binary solvent system (solvent A: 0.1% formic acid/H₂O; solvent B: 0.1% formic acid/CH₃CN) with a ratio of A:B that varied from 97:3 (0-5 min) to 3:97 (5-18 min).

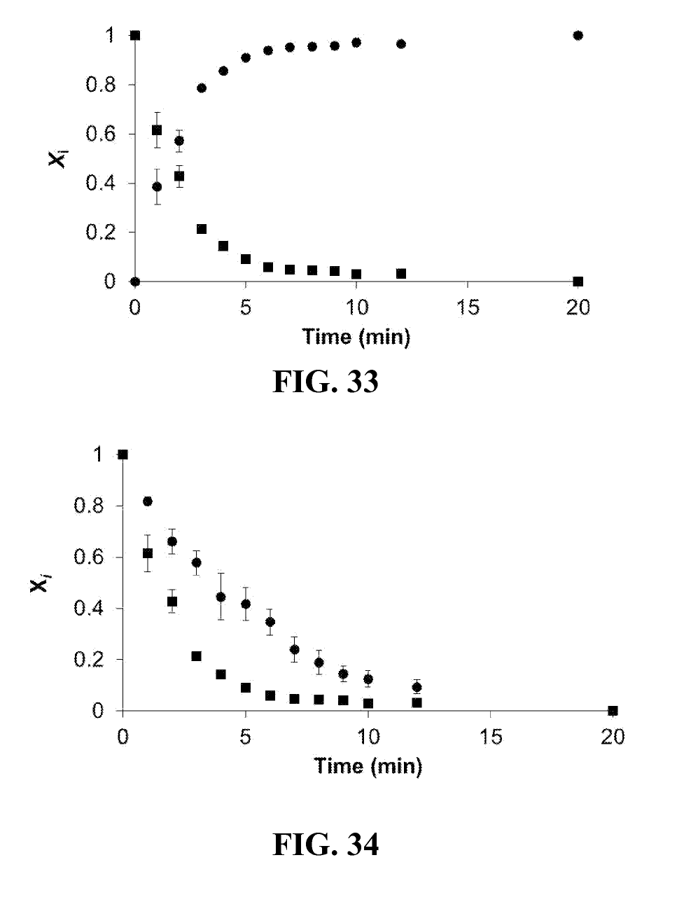

Coenzyme B₁₂ (AdoCbl) derivatives were employed. (9) The rate of photolysis at 546 nm for the TAMRA-substituted derivative, AdoCbl-1 (2.9±0.3 μM⁻¹/m), was nearly double that of its naturally occurring unlabeled counterpart, AdoCbl (1.7±0.2 μM⁻¹/m) (FIGS. 32-34), evidencing that, without being bound by theory or mechanism, excitation of TAMRA played a role in augmenting photolysis. AdoCbl-conjugates were then prepared containing long wavelength fluorophores, including SulfoCy5 (AdoCbl-2), Atto725 (AdoCbl-3), and Dylight800 (AdoCbl-4), to determine if photocleavage of the Co-alkyl link can be induced at wavelengths beyond which the cobalamin moiety absorbs (i.e. >600 nm).

It has been shown that photolysis of AdoCbl produces adenosine, adenosine-5′-aldehyde, and 5′-peroxyadenosine. (9) LC/MS confirmed that 546 nm irradiation of AdoCbl and AdoCbl-1 produces these products (Table 6). By contrast, both AdoCbl and AdoCbl-1, which only absorb light at <600 nm, were resistant to photolysis at wavelengths beyond 600 nm (Tables 7 and 8). AdoCbl-2, with an appended SulfoCy5 (λₑₓ 650 nm), produced adenosine products when exposed to 546 and 646 nm, but was unaffected by 730 and 780 nm light (Table 9). AdoCbl-3 (Atto725, λₑₓ 730 nm) delivered the adenosine photolysis products upon exposure to 546, 646, and 730 nm, but not at 780 nm (Table 10). In addition, although AdoCbl-3 suffered complete photolysis at 546 and 730 nm, there remained a significant amount of starting material upon exposure to 646 nm. The observed partial photolysis may be consistent with the fact that the Atto725 contained within AdoCbl-3 has a minor shoulder absorption in the 646 nm region. Finally, the Dylight800 (λₑₓ 780 nm)-containing AdoCbl-4 generated the expected adenosine products in response to irradiation at 546, 730, and 780 nm (Table 11). Partial photolysis was observed with 730 nm, and may be due to the shoulder absorption of Dylight800. AdoCbl-4 was resistant to photolysis at 646 nm due to the lack of absorption at this wavelength. Thus, for these exemplary embodiments the photolytic release of compounds attached to the Co of cobalamin was tunable, based on the excitation spectrum of the fluorophore appended to the ribose 5′-OH.

Tables 2-12 shows the characteristics of compounds wherein of a fluorophore appended to the ribose 5′-OH of an alkylcobalamin.

TABLE 2
Cbl-1 conjugate (10 μM) stored in the dark and photolyzed
(Xe flash lamp) at 546 ± 10 nm for 20 min.

| Photolysis | Retention | | Designated |
|---|---|---|---|
| Conditions | Time (min) | Observed Mass | Structure |
| Dark | 12.2 | 900.7 (M²⁺), 600.8 (M³⁺) | Cbl-1 |
| | 10.0 | 665.1 (M²⁺) | hydroxocobalamin |
| 546 nm | 12.8 | 486.2 (M⁺) | TAMRA-1 |
| | 13.1 | 504.2 (M⁺) | TAMRA-2 |
| | 13.7 | 470.2 (M⁺) | TAMRA-3 |

TABLE 3
Cbl-3 (10 μM) stored in the dark and photolyzed using a
Xe flash at 646 ± 10 nm for 20 min.

| Photolysis | Retention | Observed | |
|---|---|---|---|
| Conditions | Time (min) | Mass | Designated Structure |
| Dark | 11.6 | 1013.6 (M²⁺), | Cbl-3 |
| | | 676.4 (M³⁺) | |
| 646 nm | 9.7 | 665.0 (M²⁺) | hydroxocobalamin |
| | 11.8 | 712.3. (M⁺) | SulfoCy5-1 |
| | 12.0 | 730.2 (M⁺) | SulfoCy5-2 |
| | 12.4 | 696.3 (M⁺) | SulfoCy5-3 |

TABLE 4
Cbl-4 (10 μM) stored in the dark and photolyzed using a Xe flash
lamp at 730 ± 10 nm for 150 min.

| Photolysis | Retention | | Designated |
|---|---|---|---|
| Conditions | Time (min) | Observed Mass | Structure |
| Dark | 12.3 | 893.2 (M²⁺), 595.9 (M⁺) | Cbl-4 |
| 646 nm | 9.7 | 665.0 (M²⁺) | hydroxocobalamin |
| | 13.3 | 473.3 (M⁺) | Atto725-1 |
| | 13.6 | 489.3 (M⁺) | Atto725-2 |
| | 14.2 | 455.2 (M⁺) | Atto725-3 |

TABLE 5
Cbl-5 (10 μM) stored in the dark and photolyzed using a
Xe flash lamp at 780 ± 10 nm for 3 h.

| Photolysis | Retention | | Designated |
|---|---|---|---|
| Conditions | Time (min) | Observed Mass | Structure |
| Dark | 12.9 | 1139.7 (M²⁺), | Cbl-5 |
| | | 760.4 (M³⁺) | |
| 780 nm | 9.9 | 665.0 (M²⁺) | hydroxocobalamin |

TABLE 6
Cbl-6 (20 μM) after being stored in the dark and after being photolyzed
using a Xe flash lamp at 700 ± 10 nm for 2 h.

| Photolysis | Retention | | Designated |
|---|---|---|---|
| Conditions | Time (min) | Observed Mass | Structure |
| Dark | 11.6 | 1179.9 (M²⁺), | Cbl-6 |
| | | 787.0 (M³⁺) | |
| 700 nm | 9.794 | 665.0 (M²⁺) | hydroxocobalamin |
| | 10.581 | 1061.0 (M⁺), | Alexa700-2 |
| | | 531.3 (M²⁺), 264.2 (M⁴⁺) | |

TABLE 7
AdoCbl (10 μM) stored in the dark and photolyzed using a
Xe flash lamp at 546 ± 10 nm (20 min), 646 nm (20 min),
730 nm (150 min), and 780 nm (3 h).

| Photolysis | Retention | | Designated |
|---|---|---|---|
| Conditions | Time (min) | Observed Mass | Structure |
| Dark | 10.4 | 790.4 (M²⁺), 527.4 (M³⁺) | AdoCbl |
| 546 nm | 2.6 | 284.1 (M⁺) | Adenosine-1 |
| | 5.5 | 284.1 (M⁺) | Adenosine-2 |
| | 9.6 | 664.9 (M²⁺) | hydroxocobalamin |
| 646 nm | 10.4 | 790.4 (M²⁺), 527.4 (M³⁺) | AdoCbl |
| 730 nm | 10.5 | 790.4 (M²⁺), 527.4 (M³⁺) | AdoCbl |
| 780 nm | 10.4 | 790.4 (M²⁺), 527.4 (M³⁺) | AdoCbl |

TABLE 8
AdoCbl-1 (10 μM) stored in the dark and photolyzed using a
Xe flash lamp at 546 ± 10 nm (20 min), 646 nm (20 min),
730 nm (150 min), and 780 nm (3 h).

| Photolysis | Retention | | Designated |
|---|---|---|---|
| Conditions | Time (min) | Observed Mass | Structure |
| Dark | 11.7 | 1039.9 (M²⁺), | AdoCbl-1 |
| | | 520.4 (M³⁺) | |
| 546 nm | 2.5 | 284.1 (M⁺) | Adenosine-1 |
| | 5.4 | 284.1 (M⁺) | Adenosine-2 |
| | 11.6 | 914.3 (M²⁺), 609.8 (M³⁺) | hydroxocobalamin |
| 646 nm | 11.7 | 1039.9 (M²⁺), | AdoCbl-1 |
| | | 520.4 (M³⁺) | |
| 730 nm | 11.7 | 1039.9 (M²⁺), | AdoCbl-1 |
| | | 520.4 (M³⁺) | |
| 780 nm | 11.7 | 914.3 (M²⁺), 609.8 (M³⁺) | AdoCbl-1 |

TABLE 9
AdoCbl-2 (10 μM) stored in the dark and photolyzed using a
Xe flash lamp at 546 ± 10 nm (20 min), 646 nm (20 min),
730 nm (150 min), and 780 nm (3 h).

| Photolysis | Retention | | Designated |
|---|---|---|---|
| Conditions | Time (min) | Observed Mass | Structure |
| Dark | 11.6 | 1152.7 (M²⁺), 768.8 (M³⁺) | AdoCbl-2 |
| 546 nm | 2.5 | 284.0 (M⁺) | Adenosine-1 |
| | 4.9 | 284.1 (M⁺) | Adenosine-2 |
| | 11.4 | 1027.4 (M²⁺), 684.9 (M³⁺) | B₁₂ₐ-SulfoCy5 |
| 646 nm | 2.5 | 284.1 (M⁺) | Adenosine-1 |
| | 5.3 | 284.1 (M⁺) | Adenosine-2 |
| | 11.5 | 1027.0 (M²⁺), 685.2 (M³⁺) | B₁₂ₐ-SulfoCy5 |
| 730 nm | 11.6 | 1152.5 (M²⁺), 768.8 (M³⁺) | AdoCbl-2 |
| 780 nm | 11.6 | 1152.7 (M²⁺), 768.8 (M³⁺) | AdoCbl-2 |

TABLE 10
AdoCbl-3 (10 μM) stored in the dark and photolyzed using a
Xe flash lamp at 546 ± 10 nm (20 min), 646 nm (20 min),
730 nm (150 min), and 780 nm (3 h).

| Photolysis | Retention | | Designated |
|---|---|---|---|
| Conditions | Time (min) | Observed Mass | Structure |
| Dark | 11.8 | 1032.3 (M²⁺), 688.5 (M³⁺) | AdoCbl-3 |
| 546 nm | 2.4 | 284.1 (M⁺) | Adenosine-1 |
| | 12.3 | 906.8 (M²⁺), 604.8 (M³⁺) | B₁₂ₐ-Atto725 |
| 646 nm | 2.6 | 284.1 (M⁺) | Adenosine-1 |
| | 12.0 | 1032.3 (M²⁺), 688.5 (M³⁺) | AdoCbl-3 |
| | 12.2 | 907.4 (M²⁺), 605.0 (M³⁺) | B₁₂ₐ-Atto725 |
| 730 nm | 2.4 | 284.1 (M⁺) | Adenosine-1 |
| | 4.4 | 284.1 (M⁺) | Adenosine-2 |
| | 12.3 | 906.7 (M²⁺), 604.8 (M³⁺) | B₁₂ₐ-Atto725 |
| 780 nm | 11.9 | 1032.4 (M²⁺), 688.5 (M³⁺) | AdoCbl-3 |

TABLE 11
AdoCbl-4 (10 μM) stored in the dark and photolyzed using a
Xe flash lamp at 546 ± 10 nm (20 min), 646 nm (20 min),
730 nm (150 min), and 780 nm (3 h).

| Photolysis | Retention | | Designated |
|---|---|---|---|
| Conditions | Time (min) | Observed Mass | Structure |
| Dark | 12.8 | 1279.6 (M²⁺), 853.3 (M³⁺) | AdoCbl-4 |
| 546 nm | 2.4 | 284.0 (M⁺) | Adenosine-1 |
| | 4.9 | 284.0 (M⁺) | Adenosine-2 |
| | 12.5 | 1153.5 (M²⁺), 769.0 (M⁺) | B₁₂ₐ-Dylight800 |
| 646 nm | 12.8 | 1279.0 (M²⁺), 853.0 (M³⁺) | AdoCbl-4 |
| 730 nm | 2.4 | 284.0 (M⁺) | Adenosine-1 |
| | 4.7 | 284.1 (M⁺) | Adenosine-2 |
| | 12.6 | 1154.2 (M²⁺), 769.2 (M³⁺) | B₁₂ₐ-Dylight800 |
| 780 nm | 2.1 | 284.0 (M⁺) | Adenosine-1 |
| | 12.4 | 1154.2 (M²⁺), 769.2 (M³⁺) | B₁₂ₐ-Dylight800 |

TABLE 12
Cbl-7 (20 μM) stored in the dark and photolyzed using a
Xe flash lamp at 646 ± 10 nm for 2 h.

| Photolysis | Retention | | Designated |
|---|---|---|---|
| Conditions | Time (min) | Observed Mass | Structure |
| Dark | 14.0 | 958.7 (M²⁺), 639.5 (M³⁺) | Cbl-7 |
| 646 nm | 9.6 | 665.0 (M²⁺) | hydroxocobalamin |
| | 16.9 | 586.3 (M⁺) | Bodipy650-3 |

The following example demonstrates that active agents can be conjugated to exemplary compounds and can then be released from the compound in an active form. Specifically, bioreagents that are biochemically/biologically inert until activated by light can be prepared by covalently modifying a functional group for biological activity (e.g. conversion of a critical hydroxyl group to a nitrobenzyl ether). The potential application of cobalamin alone can be limited since photolysis of the Co-alkyl linkage generates functional groups (alkyl, aldehyde, hydroperoxide) not commonly required for biological activity. However, given the compartmentalized nature of cells, bioactive compounds can be converted from an inactive to an active form by altering their subcellullar location using light. For example, sequestering a cytotoxic agent that targets the mitochondria to some other cellular site should interfere with its toxicity. Subsequent photolysis would enable the cytotoxic agent to migrate to its intracellular site of action and thereby elicit cell death.

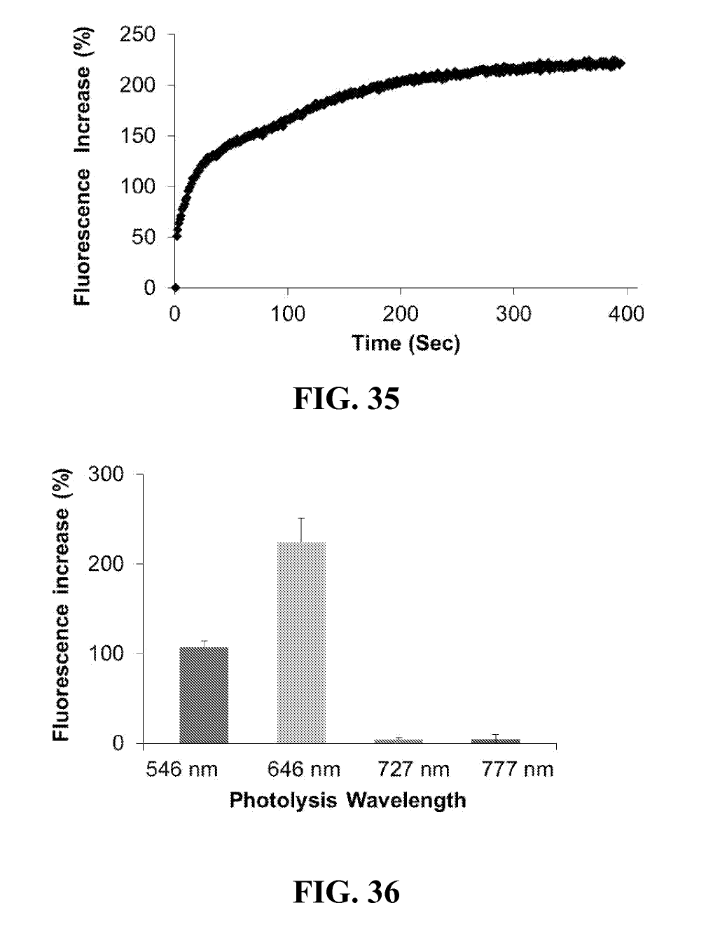

In this example, Cbl-7 was prepared, which contained an appended BODIPY® 650 fluorophore that is cytotoxic by virtue of a mitochondria-based mechanism. Since cobalamin derivatives can be taken up by and retained in endosomes, the appended BODIPY® 650 moiety, the potential therapeutic agent, was endosomally entrapped. Similar to other embodiments described herein, Cbl-7 underwent photolysis at the wavelength absorbed by the appended fluorophore (646 nm in a spectrofluorimeter), producing a corresponding increase in fluorescence (220±30%, Table 1, FIG. 35). Based on its excitation spectrum, Cbl-7 is impervious to wavelengths beyond 700 nm (FIG. 36). LC/MS data revealed that 646 nm light primarily generated the alkyl derivative BODIPY® 650-3 (FIG. 12. Scheme S7, Table 12).

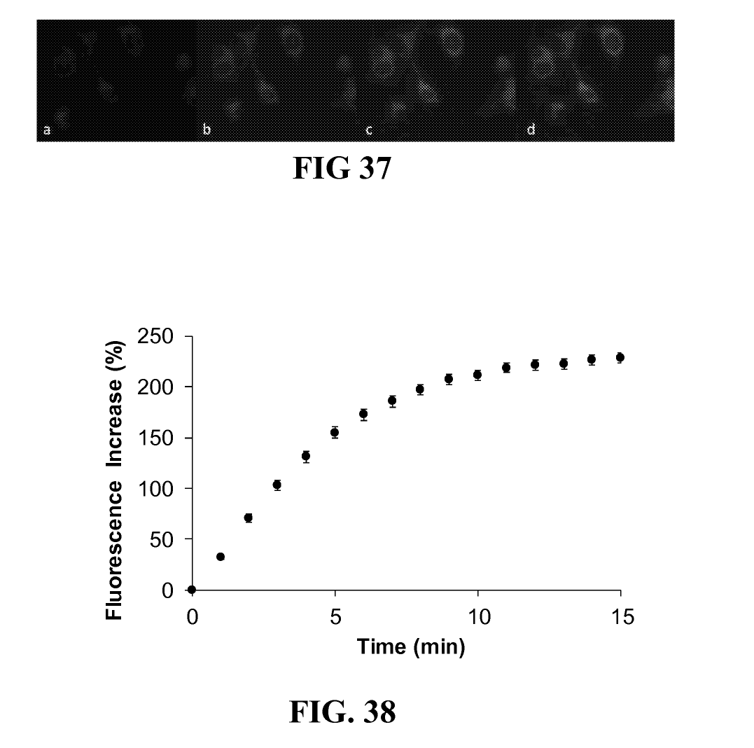

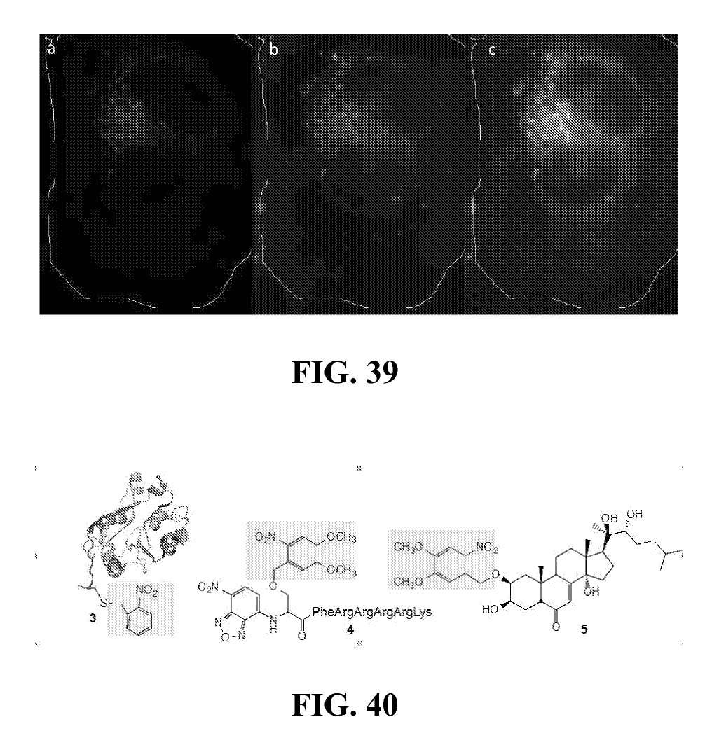

Cbl-7 accumulated in endosomes as demonstrated using rhodamine B-dextran, an endosomal marker (Mander's coefficient 0.77). Indeed, even after 5 hours in the dark, Cbl-7 was retained by endosomes (FIG. 39). Illumination of cells containing Cbl-7 with 650 nm light furnished a fluorescent increase (230±6%, FIGS. 37-38) similar to that observed in the spectrofluorimeter (220±30%). In addition, 650 nm light promoted the transfer of BODIPY® 650 fluorescence from endosomes to mitochondria, as assessed by the mitchondrial marker, Mitotracker® Green (Mander's coefficient: 0.97).

This example describes that far-red and near IR fluorophores can be used to control biological activity in a wavelength-selective fashion.

The optical window of tissue consists of the visible and near IR spectrum (600-1000 nm), where light has its maximum tissue penetration. The region <600 nm is obscured by hemoglobin in the circulatory system and melanin in the skin, whereas water interferes with light penetrance >1000 nm. These considerations play an important role in the design of reagents for intravital imaging, which can furnish information at the cellular and biochemical levels in live animals. (Pittet 2011)

A host of far-red and near IR fluorophores with optimized properties (photo-stability, brightness, water solubility) is commercially available, (Owens 1996) some of which have found clinical use. (Ovens 1996; East 2009; Kiesslich 2003) Furthermore, the size of the tissue optical window offers additional opportunities: “The large optical imaging window from ˜600 to 1000 nm enables the use of multiple fluorescent probes in a single experiment without significant bleed through between the imaging channels . . . multichannel imaging has great potential to facilitate the observation of multiple targets or prognostic indicators simultaneously, ultimately resulting in improved disease diagnosis”. (Hilderbrand 2010)

In marked contrast to the progress and opportunities that have been established for optical tissue window imaging, the use of light to control biological activity remains restricted to single reagents generally using short visible wavelengths. A strategy has recently been described that employs far-red and near IR light to control biology. (Shell 2014) Furthermore, the strategy takes advantage of the plethora of available far-red and near IR fluorophores, thereby allowing the investigator to independently control the action of multiple bio-agents in a predetermined wavelength-specific fashion.

Biological Applications of Light-Activated Agents range from biochemical to biomedical, including light-mediated enzyme inhibitors and sensors, anticancer therapy, biomaterials, and diagnostics. (Lee 2009; Lawrence 2005) For example, light-activatable analogues of enzyme sensors and inhibitors, activators of gene expression, and proteins were prepared to interrogate intracellular spatiotemporal events. (Dai 2007; Veldhuyzen 2003; Wang 2006; Wood 1998; Lin 2002; Singer 2005) Three representative examples illustrate the biological utility of light-responsive agents: (a) A light-activatable form of cofilin demonstrated that cofilin serves as a component of the steering wheel of the cell; (Ghosh 2002; Ghosh 2004) (b) a light-activated protein kinase C (PKC) sensor revealed that PKC β is active during prophase and is essential for entry into metaphase; (Dai 2007) (c) a light-activated gene expression system based on the natural product ponasterone 5 unraveled the dynamic behavior of genetic control in single cells. (Singer 2005; Larson 2013)

The nitrobenzyl moiety serves as the photo-removable functionality, as illustrated in FIG. 40 (3-5). Although a number of other photo-responsive groups have been introduced in the decades since the light-sensitive nitrobenzyl-modified ATP analog was reported, nitrobenzyl derivatives remain the standard photo-cleavable group used for the construction of photo-responsive agents. Recently, two-photon technology has been brought to bear in this field of endeavor. (Aujard 2006; Gug 2008) Certain photo-cleavable chromophores can combine the energies of two simultaneously absorbed (<1 fs) long wavelength photons, offering the opportunity of using near IR light (e.g. >700 nm) to drive an otherwise short wavelength (350 nm) phenomenon. However, two-photon technology has its challenges. First, the two-photon cross-section is restricted to a narrow plane, which limits the amount of material that can be photo-released and the size of the application area. More importantly, and as noted in a recent review, the acquisition of two photon sensitive protecting groups that can be both efficiently photolyzed and used under biological conditions “remains elusive”. (Bort 2013))

Photolysis of Organocobalamins. It has been more than 50 years since Barker and his coworkers described the photosensitivity of coenzyme B₁₂ (6; where R=5′-deoxyadenosyl or H). (Barker, 1958) Since then, a wide variety of alkylated-cobalamins (alkylated-Cbl) have been reported. (Dolphin, 1964)

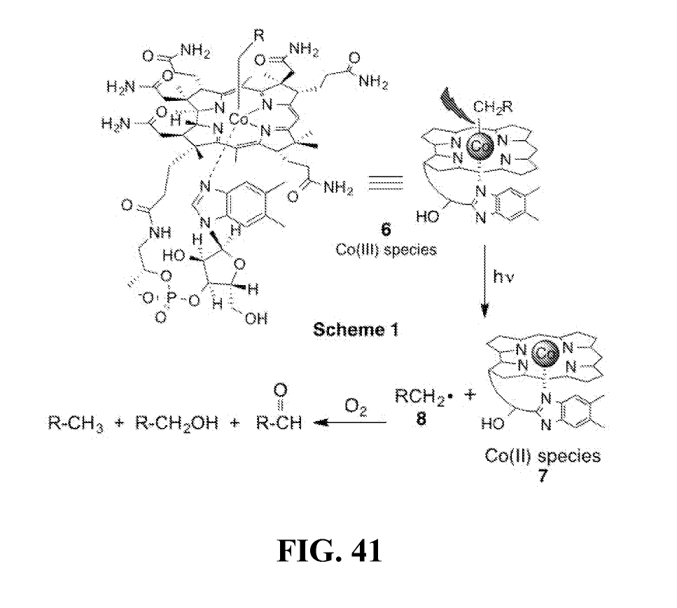

Light-induces homolytic cleavage of the Co³⁺-alkyl bond, initially furnishing Cbl(Co⁺²) 7 and alkyl radical 8 products (Scheme 1 of FIG. 41). The latter subsequently forms alkyl, alcohol, and/or aldehyde derivatives, whereas the Cbl(Co²⁺) will combine with water to generate Cbl(Co³⁺)(OH). This study confirms these products where RCH₂=adenosyl as well as for a variety of other substituents. Cbls, and their alkylated counterparts, absorb light in the 340-380 nm range, at ˜420 nm, and broadly from 500 to 560 nm. Illumination at any of these wavelengths induces Co-alkyl bond scission with high quantum yields (0.1-0.4). (Taylor 1973) Previously, the light sensitivity of Co-alkyl bond has not been used to create light-activatable bioactive compounds.

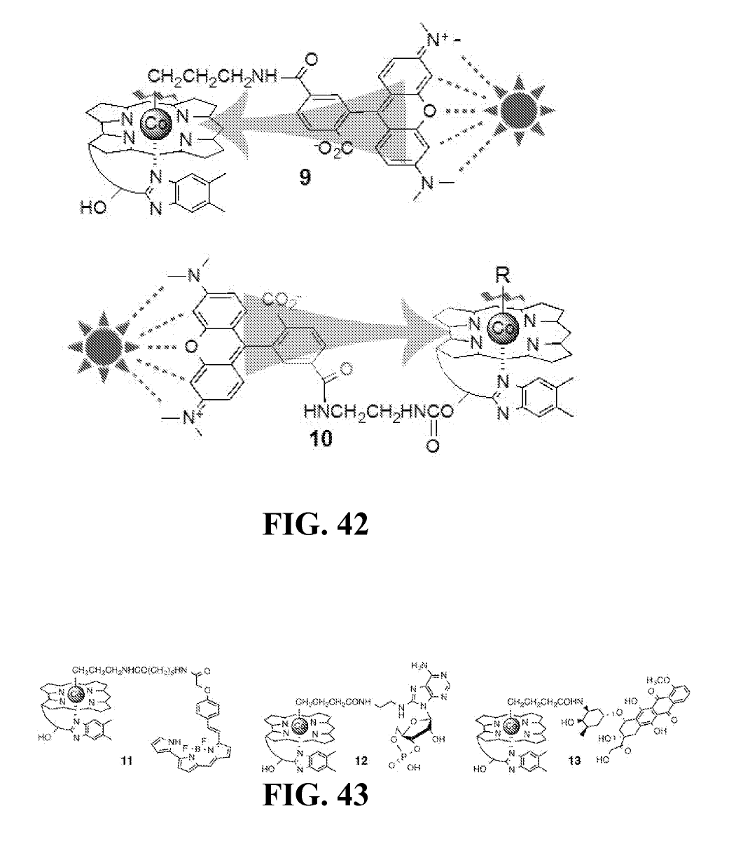

Far-Red and Near IR Photolysis of Organo-Cobalamins. The Co³⁺-alkyl bond is weak (<30 kcal/mol), suggesting that wavelengths beyond which the corrin ring absorbs (>560 nm) should be energetically sufficient for photo-cleavage. Indeed, calculations indicate that wavelengths as long as 1100 nm possess the energy required to cleave the Co—C bond in organo-Cbls. Can “antennas” be appended to Cbl for capturing and transferring the energy associated with long wavelength light to the Co-corrin ring system? The “antenna” hypothesis is explored by appending a variety of fluorophores to the alkyl chain attached to Co (9) and to the ribose ring hydroxyl moiety (10) (FIG. 42) (Shell 2014):

(1) All Cbl-fluorophore derivatives undergo photolysis at the excitation wavelengths of the appended fluorophores, including those containing TAMRA (546 nm, 9/10), sulfoCy5 and BODIPY® 650 (646 nm), Alexa Fluor® 700 (700 nm), ATTO 725 (727 nm), and Dylight® 800 (777 nm). In short, the photolytic wavelength is readily encoded based on the excitation spectrum of commercially available fluorophores.
(2) As a consequence of (1), it is possible to selectively photolyze individual compounds from mixtures of up to four different organo-Cbl derivatives via illumination at appropriate wavelengths.
(3) A variety of R groups (10) can be photo-released, including bio-active species as described below.
Photo-Release of Bio-Active Species from Cobalamins.

Light-responsive small molecules are commonly prepared by modifying a functional group essential for biological activity with a photo-cleavable moiety. However, a more general approach for constructing photo-responsive compounds is feasible with Cbls. Cbl-conjugates are either cell-impermeable or, in the case of cancer cells, are taken up by and retained in endosomes. (Bagnato 2004; Gupta 2008) Endosomal sequestration and/or cellular impermeability both have the same effect: the active species is unable to interact with its intended intracellular target. With this in mind, three Cbl-conjugates were prepared and examined (Shell 2014):

Cbl-BODIPY® 650 11: BODIPY® 650 is a mitochondrial toxin, (Kamkaew 2013; Awuah 2012) but the conjugate is non-toxic in the dark. Illumination at 650 nm rapidly initiates trafficking of BODIPY® 650 to the mitochondria in cancer cells.

Cbl-cAMP 12: cAMP has a profound effect on the cytoskeleton of the cell, but conjugate 12 is inactive on REF52 fibroblast behavior in the dark. Illumination induces the loss of stress fibers, cell shrinkage and rounding, known consequences of the cAMP-dependent protein kinase signaling pathway. (Oishi 2012)

Cbl-doxorubicin 13: Doxorubicin is a widely used anticancer agent that displays off target cardiotoxicity. (Patil 2008; Volkova 2011) The cytotoxicity of this conjugate in HeLa cells was examined as a function of illumination time. Light-only treatment, or exposure to 13 in the absence of photolysis, has no effect on cell viability. By contrast, increasing illumination time in the presence of 13 furnishes a light dose-dependent increase in cell death that ultimately recapitulates that produced by doxorubicin alone.

Summarily, then, far-red and near IR fluorophores enjoy widespread clinical applications. These fluorophores can be used to control biological activity in a wavelength-selective fashion, affording spatiotemporal control over multiple photo-responsive species as described in the next example.

This example relates to wavelength-encoded, photo-responsive molecular constructs. The scope and limitations of cobalamin-based photo-responsive constructs is examined. Sets of wavelength-controlled cobalamin-drug conjugates that are active in the red, far-red, and near-IR are acquired. In addition, the structural features that promote thiolatocobalamin stability in the dark and photo-cleavage upon illumination are identified. The application of thiolatocobalamins as carriers of peptide therapeutics is evaluated.

This study focuses on the scope and limitations of Cbl-based photo-responsive constructs to identify sets of orthogonally-controlled, wavelength-responsive constructs and examine the photochemistry of thiolatocobalamins, which will be used for the delivery of therapeutic peptides.

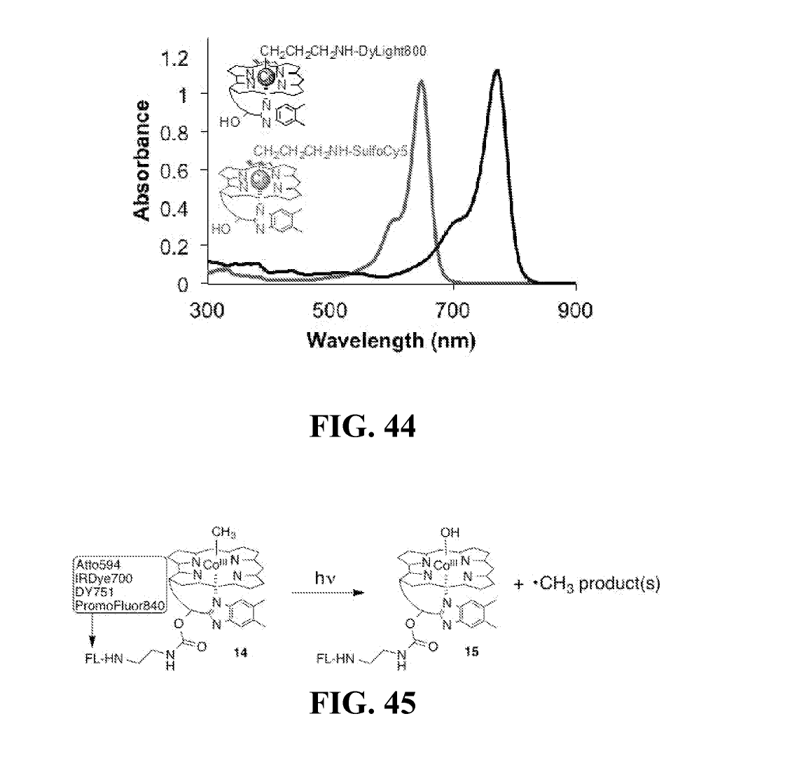

Wavelength-Encoding: Exploration of Orthogonal Control. As noted in the previous example(s), four different species were photolyzed by sequential illumination from long to short wavelength i.e. 777 nm, 700 nm, 646 nm, 546 nm. (Shell 2014) For the four fluorophores used in this particular experiment, sequential photolysis was required for selective activation because the longer wavelength fluorophores absorbed light in the region where the shorter wavelength fluorophores are excited. Sequential illumination is sufficient if there is a desired sequence of photo-initiated events. However, complete orthogonal control provides sequence independence and thus greater flexibility in terms of biological regulation. A non-interfering orthogonal pair of photo-responsive constructs is identified: Cbl-SulfoCy5 and Cbl-DyLight® 800 are photochemically distinct at 646 nm and 777 nm (FIG. 44). Consequently, they can be individually photo-manipulated without the need to resort to a specific illumination sequence. A goal of this work is to establish a quantitative measure of photolytic release and to identify tri- and tetra-orthogonal groups of wavelength-specific responders. The biomedical rationale is discussed in the further examples below.

There is a wide variety of red, far red, and near IR commercially available fluorophores: the PromoFluors (Promokine), the DYs (Dyomics), the ATTOs (Atto-TEC), the HiLyte® Fluors (AnaSpec), the Alexa Fluors® (Invitrogen), the DyLights® (Pierce), etc. A summary of the photophysical properties of many of these fluorophores can be found at the fluorophores.org website.

Although space limitations preclude a detailed discussion of the numerous possibilities, two examples are furnished to illustrate the strategy that is to be undertaken. Tri-orthogonal group: ATTO 594 (λₑₓ 602 nm), IRDye700DX (λₑₓ 689 nm), and Promo-Fluor-840 (λₑₓ 843 nm). Tetra-orthogonal group: ATTO 594 (λₑₓ 602 nm), IRDye700DX (λₑₓ 689 nm), DY-751 (λₑₓ 751 nm), and Promo-Fluor-840 (λₑₓ 843 nm). The fluorophores were chosen based on their non-overlapping excitation wavelengths. The methyl-Cbl derivatives (14) containing these fluorophores are synthesized as described (FIG. 45). (Shell 2014)

Wavelength-directed, compound-specific photo-release will be assessed for selectivity, which is arbitrarily set at 20-fold. Fluorophore-Cbls that fail to meet this standard will be replaced with other commercially available fluorophores.

Although the absorbance/excitation spectra serve as a guide for predicting photosensitivity at any particular wavelength, such variables as the extinction coefficient of the fluorophore, the efficiency of energy transfer from fluorophore to Cbl, and quantum yield (Φ), will contribute to the extent by which two or more fluorophore-Cbls can be distinguished. Photolysis rates are to be acquired as a function of wavelength for all compounds (14) in order to furnish a quantitative assessment of wavelength selectivity amongst these derivatives. Product formation rates are readily assessed via absorbance spectroscopy; the spectrum of the photolyzed product (15) significantly differs from that of the starting alkyl-Cbl 14 (FIG. 45).

Second, the Φ at the designated wavelengths of the lead fluorophore-substituted conjugates (14) can be determined Φs can provide a quantitative measure of any drop-off in photo-sensitization/photo-release as a function of wavelength. A Φ is typically determined via simultaneous photolysis of a standard (“chemical actinometer”), however both the K₃[Fe(C₂O₄)₃] (250-500 nm) and meso-diphenylhelianthrene (475-610 nm) standards lack the wavelength bandwidth that our technology requires. A direct assessment of Φs for alkyl-Cbls is developed via illumination of the sample with a measured irradiance from one direction and quantification of the photolytic product (15) at 90° relative to the illumination source.

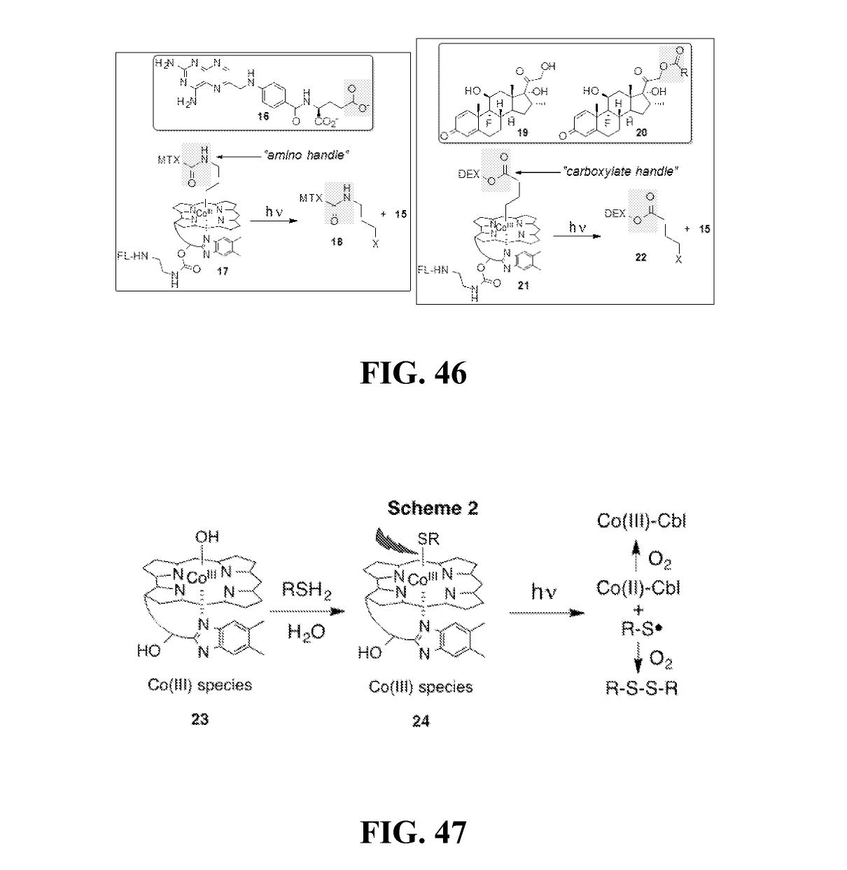

The wavelength-dependent photo-release of Cbl derivatives (17 and 21) of methotrexate (16) and dexamethasone (19) can also be assessed (FIG. 46). Both drugs are routinely used for the treatment of RA. The highlighted carboxylate in (16) is not required for activity and a variety of substituents (including peptides, antibodies, and polymers) have been conjugated to this position (Majumdar 2012; Wang 2007; Everts 2002). Most relevant to this discussion are the array of anti-inflammatory N-alkyl carboxamide MTX derivatives that are analogous/identical to the expected photolyzed products (18) (X═H, OH). (Heath 1986; Rosowsky 1986; Piper 1982; Rosowsky 1981; Szeto 1979). DEX (19) is also pharmaceutically available as the acetate (20) (R=Me) that, like many other short chain acylated DEX derivatives (e.g. 22), is designed to promote skin/ocular permeability (Markovic 2012; Civiale 2004) or serve as a sustained release version when injected as an intramuscular depot due to its low water solubility (Samtani 2005). Wavelength discriminable versions of (17) and (21) will be assessed in buffer, via LC-MS and in the subsequent Example cell-based studies, via commercial ELISA kits. (17) and (21) are not designed to be active or inactive prior to or after photo-release from the Cbl (FIG. 46).

Photo-Responsive Thiolatocobalamins. Anti-inflammatory peptide-based agents have received significant attention. (Luger 2007; Bohm 2012) Although it is certainly possible to couple a peptide to a Cbl-amino handle, as in (17), or a Cbl-carboxyl handle, as in (20), the likely presence of multiple nucleophiles or electrophiles on a given peptide framework could make such a synthetic approach cumbersome. With this in mind, the preparation and properties of thiolatocobalamins (thiolato-Cbl) is explored.

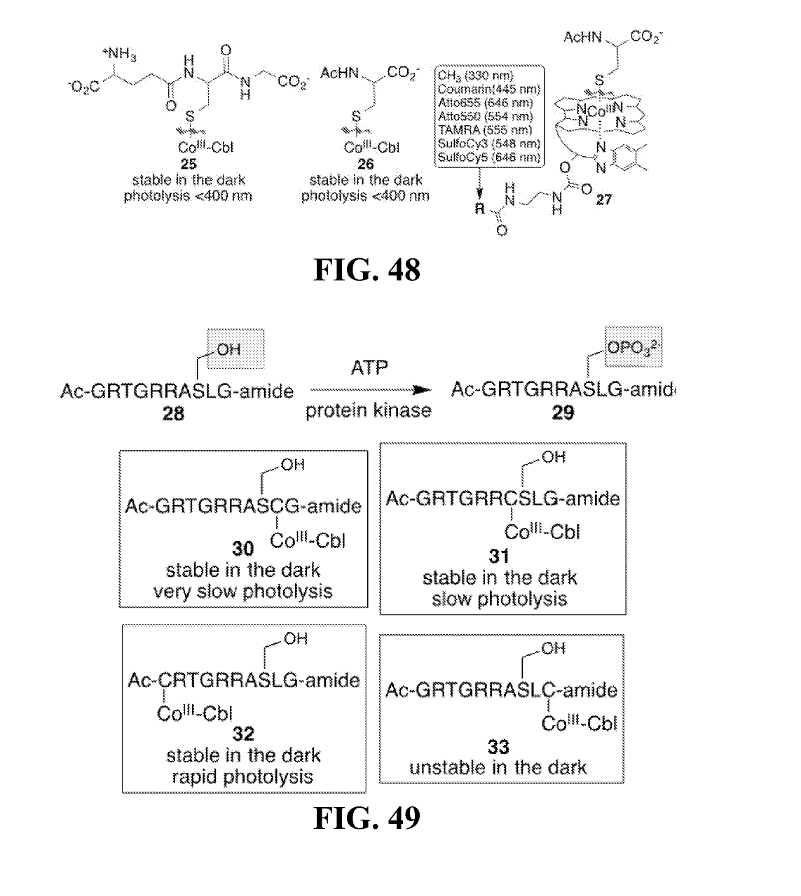

Thiolato-Cbls (24) are easy to prepare: simply expose mercaptans to (23) under neutral, aqueous, aerobic conditions (FIG. 47, Scheme 2). Glutathione-Cbl (25) is one of the primary intracellular forms of vitamin B₁₂. (Pezacka 1990; Brasch 1999) A few other thiolato-Cbls have been described, including N-acetylCys (26) (FIG. 48). (Pezacka 1990; Brasch 1999)

Photolysis in air produces the Co(II)-Cbl product, which is oxidized to the Co(III) species, and a thiyl radical, which is converted to a disulfide or oxidized product (FIG. 47, Scheme 2). (Tahara 2013) The photo-cleavage of fluorophore-substituted N-acetyl Cys-Cbl derivatives (27) has been examined and found that unlabeled thiolato-Cbls (R═CH₃ in (27)) are only photolyzed at wavelengths less than 400 nm. However, fluorophore-substituted derivatives of (27) suffer photolytic cleavage at wavelengths absorbed by the fluorophore. The following questions are addressed:

### (i) What are the Structural Requirements for the Preparation of Photo-Responsive Thiolato-Cbls?

Several Cbl-Cys analogs (30-33) of a protein kinase substrate (28) have been prepared (FIG. 49). All Cbl-peptides are stable in the dark except for (33). The photo-cleavage rates for the dark stable Cbl-peptides vary significantly: (32) (12×)>(31) (2×)>(30) (1×). These results suggest that nearby functionality influences photochemical cleavage rates and dark stability. In particular, the immediate microenvironment could impact the ability of the photo-generated intimate [Co(II)Cbl/thiyl] radical pair to separate, which is known to control the photolytic yield. (Peng 2010)

Those Cys-Cbl microenvironments that ensure dark stability yet promote rapid photolytic release can be identified. This will be assessed by examining the stability and photo-responsive properties of Cbl conjugates of Ac-Xaa-Cys-Yaa-amide tripeptides. A peptide library will be prepared containing 19 different amino acids at the Xaa and Yaa positions (Cys will be excluded from Xaa and Yaa). The 361-member library, synthesized in a one-peptide-per-well format will be (i) exposed to HO—Co^{III}-Cbl to prepare the corresponding peptide-S—Co^{III}-Cbl conjugates. The (ii) conjugates are assessed for stability in the dark via absorbance spectroscopy as a function of time; and (iii) the rate of photo-cleavage as a function of wavelength (360, 440, and 550 nm). This provides information on how local structure influences Cys-Cbl dark stability/photo-responsiveness and thus identify those sequences that promote dark stability and photo-cleavage. It is possible that additional non-natural structural features can also be explored. (Lee 1999; Lee 2000; Yeh 2001)

### (ii) Are Thiolato-Cbls Susceptible to Wavelength-Directed Orthogonal Control?

Although simple thiolato-Cbls are only susceptible to short wavelength photolysis (<400 nm), they can be rendered photo-responsive at longer wavelengths by attaching fluorescent antennas (e.g. coumarins, Cy3, Atto550; see 27). Photo-responsiveness out to 550 nm is extended. An array of far-red and near IR antennas onto (27) can be inserted, where the NAcCys will be replaced with leads identified from the peptide library study. The dark stability, photo-responsiveness, and the acquisition of orthogonal wavelength-responsive sets of reagents are to be explored. Photolysis rates and Φs are to be obtained.

(iii) Can Peptide-Based Bio-Active Species be Released from Thiolato-Cbls?

Assuming that lead sequences of Xaa-Cys-Yaa are identified and that orthogonal, wavelength-directed, photo-release in the far-red/near-IR is feasible, the dark stability/photo-responsiveness of Cbl-tripeptide conjugates appended to two peptide-based anti-inflammatories can be examined: the 13 amino acid α-melanocyte stimulating hormone (α-MSH) (Getting 2009; Luger 2007) and the annexin-1 peptide fragment Ac2-26 (Yang 2013).

The biological activity of the free peptides containing the appended library identified Cys-containing tripeptides is assessed, as described herein. In the unlikely event that the peptides are biologically inactive, it may be necessary to insert spacers (e.g. Ser-Gly) between the Xaa-Cys-Yaa and the anti-inflammatory peptide sequence. Once the biological activity of the peptides is validated, fluorophore-Cbl-peptides of the general form (27) will be prepared. Dark stability and the relative rate of photolysis as a function of wavelength is noted for each of these and compared and contrasted. In the event that the photophysical properties of thiolato-Cbls are insufficient (e.g. instability in the dark, poor release upon illumination with far-red light, etc.), it should be feasible to attach peptides to an appropriately derivatized alkyl-Cbl, using bio-orthogonal chemistry such as the Huisgen reaction. (Best 2009; Kolb 2001).

This example describes wavelength-encoded drug delivery. It has been demonstrated that bio-agents can be concealed in the densely populated protein sheath of erythrocyte membranes and subsequently photo-released to generate active species, including therapeutic agents, second messengers, and enzyme sensors. This validated strategy is coupled with the wavelength-encoded constructs developed in other examples to create a new family of drug release vehicles. In addition to providing a potential means to separately control the timing and spatial release of multiple drugs, this strategy offers a possible general approach for protecting therapeutic peptides against the proteolytic environment of the blood.

Wavelength-Encoded Drug Delivery. A mix of NSAIDs, glucocorticoids, and disease-modifying antirheumatic drugs (DMARDs) are currently used to treat RA. DMARDs slow disease progression and include an array of small molecules: MTX (16) (FIG. 46), chloroquine, cyclosporine A, D-penicillamine, various gold salts, and sulfasalazine to name but a few. The “biologics” are a relatively new family of DMARDs and include the antibody-based agents Infliximab, Etanercept, Adalimumab, Certolizumab, and Golimumab. (Kukar 2009) In addition, several peptide-based agents have shown extraordinary DMARD behavior, but are limited by the pharmacokinetic properties that plague most peptides. (Luger 2007; Bohm 2012) It is not practical to create light-activatable forms of all of these drugs. Instead, a glucocorticoid (DEX, 19), a DMARD (MTX, 16), and two peptides (a-MSH and Ac2-16) are used to explore the utility of wavelength-encoded drug delivery. The approach outlined below should be applicable to many, if not most, of the drugs in the RA arsenal.

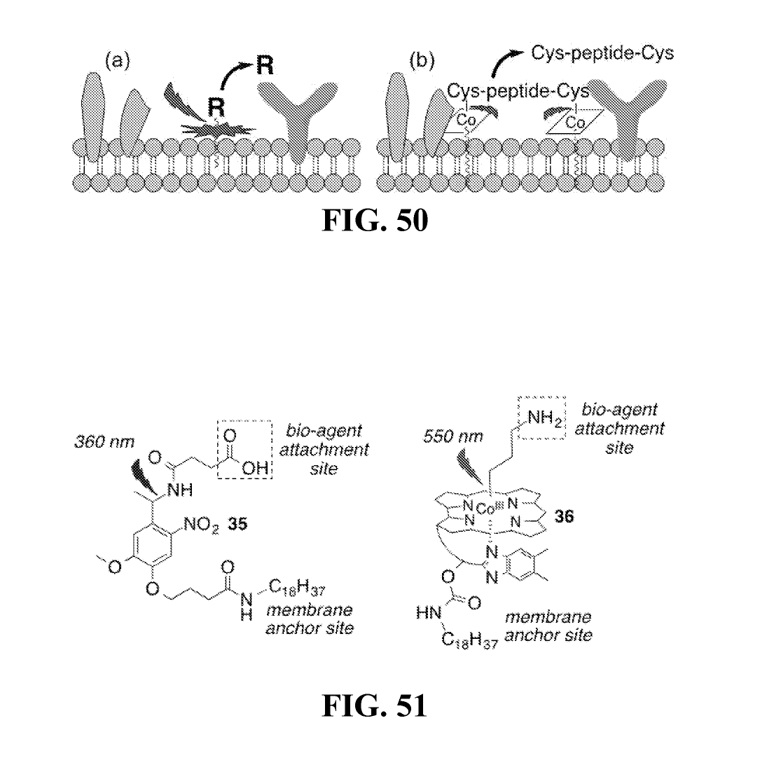

Erythrocytes as Carriers of Photo-releasable Surface-Loaded Therapeutics. The work outlined in these examples is designed to explore the photophysical properties of a new series of far-red/near-IR photo-responsive agents. Cbl is purposely installed onto bioactive agents in a fashion that should not interfere with their activity (i.e. 17, 21 and the peptides). Instead, an alternative approach was developed to control bio-activity: bio-agents are hidden in the densely populated protein sheath of cell membranes and subsequently photo-released to generate the active agents (FIG. 50). (Nguyen 2013) In this strategy, photo-cleavable groups are inserted between the concealed bio-agent and its lipid anchor. This example seeks to apply this and related strategies to construct wavelength-encoded drug delivery vehicles.

As noted previously, erythrocytes have been described as the “champions [of] drug delivery systems”. (Muzykantov 2010) Drugs, including biologics, can be easily introduced into the erythrocyte interior or attached to the cell surface. (Muzykantov 2013) As shown in FIG. 50, a surface strategy with the conventional nitrobenzyl group (35) (Nguyen 2013) (hv=360 nm) on the surface of RBCs, as well as the Cbl species (36) (hv=550 nm) to hide and photo-release protease and a protein kinase sensors (FIG. 51). These studies were performed with RBC ghosts wherein the majority of the hemoglobin was removed. However, ghosts lack the circulatory lifetime of normal erythrocytes. Wavelengths beyond the reach of hemoglobin may be employed to control photo-release so that ordinary RBCs can be used as light-responsive drug carriers.

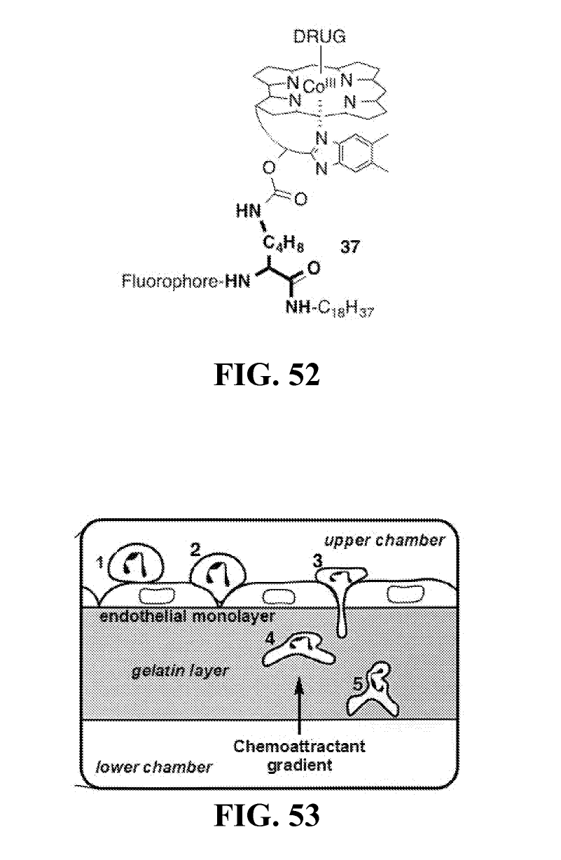

The primary needs for a FIG. 50 lipid anchor are threefold: (i) a hydrophobic moiety, and attachment sites for the (ii) fluorophore and the (iii) Cbl. Although a number of options are available, initially, a strategy analogous to one that successfully furnished the RBC membrane-embedded/photo-releasable derivatives (35) and (36) is to be employed. (Nguyen 2013; Smith Unpublished Results) The lysine derivative (37) (FIG. 52) will be prepared; the synthetic protocol (Leschke 1997) provides the requisite flexibility for using an array of fluorophores and lipids. A derivative in which fluorophore=acetyl will be used as a control to compare and contrast rates of photo-release at various wavelengths. Drugs will be attached to Cbl as amide (MTX; 17), ester (DEX; 21), thiolato-(peptides; 27) or variants thereof. The following is to be explored: (i) wavelength-dependent release of bio-active agents from RBCs and assess (ii) wavelength-dependent orthogonal control over RBC-Cbl-drug mixtures.

Although both small molecules and peptides are demonstrated that can be hidden within the protein-sheath of RBCs (Nguyen 2013), it is unclear whether larger peptides, such as α-MSH and Ac2-26, when appended via a single site to the RBC surface (FIG. 50a), will be unavailable to their biological receptors. Consequently, (iii) derivatives of the form are also to be examined: Xaa-Cys(Cbl-lipid)-Yaa-peptide-Xaa-Cys(Cbl-lipid)-Yaa. It is assumed that attachment at two sites to the RBC surface (FIG. 50b) will lay the peptide flat across the membrane, rendering it biologically unavailable until photo-released. Finally, the (iv) dark stability of these derivatives is explored, especially in the presence of plated fibroblasts (RBCs are non-adherent). Specifically, whether an undesirable transfer of the lipidated-Cbl from the RBC membrane to fibroblast membrane occurs upon incubation in the dark is examined. These experiments are conducted in the presence of serum, which contains albumin, another species that can potentially leach (37) from the RBC membrane. Should unwanted transfer of the lipidated-Cbl be observed from the RBC to other cells or soluble proteins, the C₁₈ anchor will be replaced with (a) a diacylphospholipid to enhance membrane affinity or (b) covalently append the Cbl moiety to the RBC membrane.

Erythrocytes as Carriers of Photo-releasable Interior-Loaded Therapeutics. Drugs may be loaded into the interior of erythrocytes as well. For example, RBCs have been used to continuously deliver DEX with an enhanced lifetime relative to the free drug alone (Rossi 2006). Drug loading is easily accomplished by exposing the RBCs to a hypotonic solution, which creates small pores in the membrane. Following drug uptake, an isotonic solution is applied to close the pores. This procedure is extremely mild and maintains the functional integrity of the RBCs. (Muzykantov 2010; Biagiotti 2011) The drug-containing RBCs are then re-introduced into the patient.

DEX is loaded into RBCs as DEX-21-phosphate, a non-diffusible, cell-impermeable form of the drug. DEX-21-phosphate is slowly hydrolyzed in the RBC to furnish DEX, which diffuses out of the erythrocyte. This slow release form of DEX has been involved in a variety of clinical trials, including as a therapeutic for cystic fibrosis, ataxia telangiectasia, ulcerative colitis, and Crohn's disease. (Rossi 2004; IEDAT01; Bossa 2008; Castro 2007) DEX-21-phosphate/RBC serves as a model for a potentially general strategy: the intracellular sequestration of drugs in RBCs. It is assumed that fluorophore-Cbl-drugs will not leak out of RBCs since Cbl derivatives are not membrane permeable. Upon photolysis of the drug-Cbl linkage, the cleaved drug is then free to escape the RBC.

The following questions are explored: (i) Can fluorophore-Cbl-bioagents be introduced into RBCs via hypotonic loading and retained in the dark? (ii) Can the bio-agents be released upon illumination at the appropriate wavelengths?

Further, a variety of nanotechnologies is available to serve as suitable RBC alternatives. For example, mesoporous silica nanoparticles contain hundreds of empty channels in a honeycomb arrangement that have been loaded with a variety of drugs. (Vivero-Escoto 2010; Li 2012; Coll 2013) These channels have been capped with an array of moieties, including light-cleavable species. (Croissant 2013; Mal 2003; Wan 2013) Photo-removal of the channel capping agents results in drug release. The channel diameter can be varied to encapsulate everything from small drugs to proteins. (Popat 2011) Consequently, channel capping with fluorophore-Cbls offers a means to release drugs, peptides, and proteins in a wavelength-defined fashion. Mesoporous silica nanoparticles (and other nanotechnologies) can serve as useful constructs for the application of a light-encoded strategy.

This further example describes drug-specified release via wavelength-encoding to detect on-demand site-targeted anti-inflammatory control. The efficacy of the wavelength-encoded drug delivery strategy is assessed using a multiple human cell line-based 3D model of the arterial/synovium interface. The light- and wavelength-dependent ability of certain constructs may block the expression of pro-inflammatory signals and cell adhesion molecules in cellular models of the arterial endothelium, the immune system, and the synovium. The ability of these agents to block transendothelial migration of leukocytes into a model arthritic synovium under shear flow conditions is examined. Further, whether wavelength-encoded drug delivery can be used to dispense specific therapeutics in a 3D model of the vasculature-synovial joint interface is tested.

Drug-specified release via wavelength-encoding: An assessment of on-demand site-targeted anti-inflammatory control.

The efficacy of wavelength targeted drug delivery is assessed using a multiple human cell line-based 3D model of the arthritic arterial/synovium interface. The inflamed endothelial vasculature serving the arthritic synovium releases pro-inflammatory cytokines that attract leukocytes (e.g. monocytes, CD4+ T cells). Leukocytes bind to the inflamed endothelium via cell adhesion molecules (CAMs) and subsequently migrate through the vessel wall into the synovium. Additional cellular (monocyte→macrophage) and biochemical (leukocyte release of pro-inflammatory signals) events transpire that ultimately result in damage to the components of the synovial joint. MTX and DEX block these as well as other pro-inflammatory signals/behaviors. In addition, α-MSH and related derivatives have been described as having the “sledgehammer properties of a steroid, but without the side effects”. (Getting 2009) Unfortunately, α-MSH is rapidly proteolyzed in the blood. (Catania 2004) The peptide Ac2-26 also displays impressive RA anti-inflammatory activity. (Yang 2013)

Four cell types are examined, both alone and in a 3D combination, to assess the properties of some agents: (i) HMEC-1 is an endothelial cell (EC) line that is generally considered to be one of the very best models of the vascular endothelium. Commercially available HUVECs are also used, as an alternative EC line. (ii) THP-1 is a monocyte cell line commonly used to “provide insight into the roles of the interconnection of monocytes-macrophages with other vascular cells during vascular inflammation”. In addition, monocytes (CD14+) are isolated from RA peripheral blood mononuclear cells (PBMC) using a commercial kit. (iii) T cells comprise up to 50% of synovial tissue cells, most of which are CD4+. They are likewise isolated from RA PBMCs. (iv) Human synoviocytes from RA patients is used to model the synovial cellular environment.

The photo-responsive agents in these examples are unimolecular entities that should be water-soluble (ws), whereas others are associated with carriers (RBCs or mesoporous silica). “ws” and “rbc” designates the nature of the Cbl-appended drug. For example, MTX_{ws} is a water-soluble Cbl-linked MTX derivative. The following series of experiments is performed at the (i) biochemical and (ii) cellular levels and using (iii) multi-wavelength control:

### (i) Biochemical Control:

Both HMEC-1 and THP-1 cell lines respond to inflammatory activation via the production and release of cytokines (IL-1α, IL-6, IL-8 and TNFα), the expression of cell surface CAMs (ICAM-1, VCAM-1, E-selectin), and the activation of NF-κB; biochemical responses known to be inhibited by MTX, DEX, Ac2-26, and αMSH. (Luger 2007; Everts 2002; Chan 2010; Chen 2002; Nehme 2008; Joyce 1997; Peshavariya 2013). In addition, MTX is known to promote adenosine release in HMEC-1 and lymphocytes. (Morabito 1998)

The anti-inflammatory properties of adenosine are at least partially attributed to blocking the production of CAMs. (Linden 2012) The ability of the Cbl-agents to suppress inflammatory responses in HMEC-1/HUVECs, THP-1/isolated monocytes, and lymphocytes in a wavelength-dependent fashion is examined. One example is explicitly discussed here to exemplify the experiments that are conducted.

MSH_{rbc}: it is assumed that αMSH, concealed under the protein arbor on the erythrocyte surface, is unable to interact with its receptors on other cells until photo-released (FIG. 50). In addition, the proteolytic stability of MSH_{rbc} is evaluated. In spite of the extremely promising anti-inflammatory properties of αMSH, it's half life is only a few minutes when administered by IV, due to serum proteases. (Bohm 2012; Catania 2004) It is previously reported that other peptides, when hidden in the protein sheath of RBCs, are protected from proteolysis until photo-released. (Nguyen 2013; Smith Unpublished Results) The relative proteolytic stabilities of αMSH and MSH_{rbc} (FIG. 50; single- and double-site attached) in the presence of proteases known to act on αMSH is determined. (Bohm 2012) Relative stabilities in serum is also evaluated. It is noted that it may be necessary to vary the lipid anchor sites on the peptide to ensure its biological silence and proteolytic stability. Finally, unlike the other anti-inflammatories, αMSH is known to transform CD4+ into Regulatory T cells (Tregs), which serves to abrogate the immune response and figures prominently in potential therapies for autoimmune diseases. (Wright 2011) The light-dependent MSH_{rbc} transformation of CD4+ T cells into Tregs is assessed using a previously described protocol. (Taylor 2011)

The majority of the Cbl-reagents of these examples should be bio-active prior to and after illumination (i.e. block activation of cytokines and CAM expression). DEX-Cbl_{ws} is a likely exception since it is assumed that the Cbl appendage renders the DEX substituent membrane impermeable and thus unable to bind to its intracellular receptor prior to photolysis. By contrast, it is assumed that all RBC-based Cbl-reagents are designed to be inactive until photo-released.

### (ii) Cellular Control:

A hallmark of RA is leukocyte recruitment to and accumulation in the synovium/synovial membrane. Leukocyte transendothelial migration into the arthritic synovium is mediated is by both leukocytes and endothelial cells (EC). In vitro models of this migratory behavior (and interference by drugs) commonly consist of an EC monolayer cultured on collagen gels (FIG. 53). (Muller 2008; Shulman 2009) Migration of THP-1 cells and monocytes isolated from RA patients across an activated (TNFα) EC monolayer in response to chemoattractants (e.g. RANTES, MCP-1) is quantified. The ability of DEX_{WS}, MTX_{ws}, MSH_{ws}, Ac2-26_{ws}, DEX_{rbc}, MTX_{rbc}, MSH_{rbc}, and Ac2-26_{rbc} to block transendothelial migration in the dark and when pre-illuminated is noted. The photo-release of the anti-inflammatory drugs is expected to suppress the TNFα-stimulated expression of EC CAMs/RANTES-stimulated monocytes and thereby block transendothelial migration. One example is explicitly discussed here.

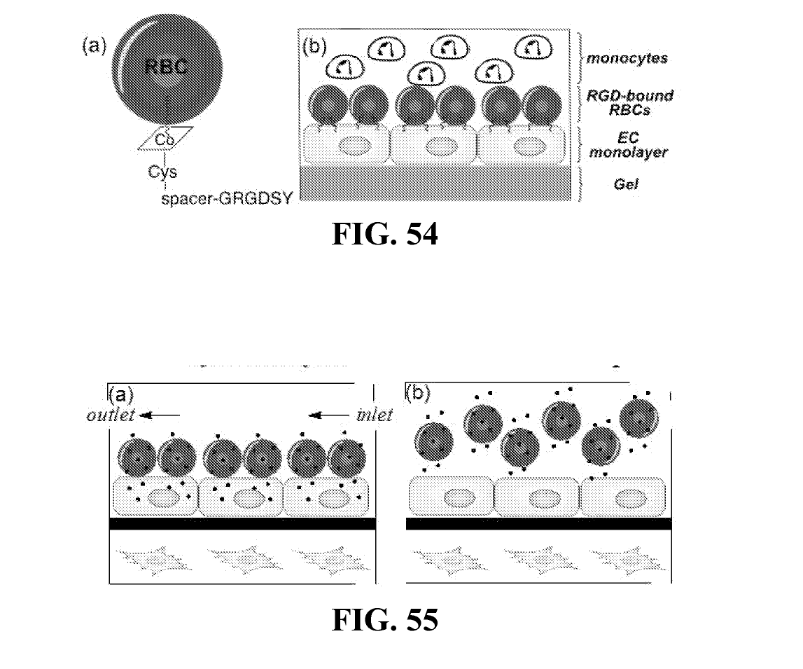

A wide variety of “RGD” peptides are described that promote cell adhesion, including several nonapeptides that target inflamed RA endothelium. (Yang 2011; Wythe 2013; Lee 2002) The GRGDSY sequence is used for the initial studies, as it is known to bind to ECs, even when appended to a polymer. (Lin 1992) Various analogues of the general structure lipid-Cbl-spacer-GRGDSY are prepared, and the lipid-peptide conjugate is inserted on the cell surface of RBCs (FIG. 54a). Then, the RBCs are introduced into endothelial monolayer migration assay system. It is noted that others have described RGD-modified RBCs and these have been shown to bind to ECs. (Fens 2010) Although these previously described RBCs were prepared by covalently attaching the RGD peptide to surface proteins, it is assumed that the lipid anchored RGD peptides described herein behave in an analogous fashion. RBC binding to the endothelial monolayer is confirmed by microscopy and by the expectation that monocyte migration into the gel layer will be blocked/impeded by the bound RBCs (FIG. 54b). Photo-cleavage of the RGD peptide from the cell surface releases the RBC from the endothelial monolayer and restores monocyte binding/migration.

Assuming that RBC/EC contact occurs as described, a transwell format is used to assess whether photo-release of anti-inflammatory drugs from RBCs in contact with ECs (FIG. 55a) has a more powerful effect than from RBCs that are free in solution (FIG. 55b). This is analyzed by comparing inflammatory protein expression levels from ECs [Biochemical control (i)] exposed to equivalent amounts of RBCs in FIG. 55a and FIG. 55b.

The ability of RBC-released anti-inflammatory agents to down-regulate the inflammatory biochemical response in activated (CD3 and CD28) T cells and in synoviocytes isolated from RA patients is also assessed. These cells are positioned in the lower chamber of transwell plates, thereby serving as a 3D model of the arthritic synovium separated from the arterial system by the endothelial barrier. Are αMSH and Ac2-26, upon RBC release, capable of traversing the endothelial monolayer and influencing inflammatory behavior in the lower chamber? These experiments are conducted in the presence of serum in the upper chamber to challenge the proteolytic stability of αMSH and Ac2-26 (cf. FIG. 55a/55b). Finally, chambers have been developed, in which the upper compartment has an inlet and outlet to mimic shear flow conditions. (Muller 2008) The flow chamber can be mounted on our microscopes, which are integrated with on-board lasers for photo-activation and digital recording for image capture.

As discussed in previous sections, water-soluble Cbl-peptides should be susceptible to proteolysis. It is noted that long-term RBC/EC contact (FIG. 55a) in small capillaries could be deleterious; therefore, RBC release after drug discharge is important.

(iii) Multi-Wavelength Control:

In some embodiments, the present disclosure provides embodiments of compounds having the ability to control the release of different anti-inflammatory agents using specific wavelengths. Initial experiments examine wavelength-specific release from mixtures of RBCs containing different drugs.

Immunoassays for the detection of DEX (Neogen Corporation) and MTX (Alpha Labs) are commercially available, and the αMSH and Ac2-26 peptides are detectable by LC-MS. Using the arthritic arterial/synovium model, an extension of the FIG. 55a strategy is pursued.

While not wishing to be bound by theory, it is believed that it is feasible to dock the RBC at the endothelium monolayer, unload the drug cargo with one wavelength and detach the RBC with a second wavelength. The lipidated RGD peptide with its photo-cleavable Cbl (lipid-Cbl-spacer-GRGDSY, FIG. 54a) should be amenable to this approach. Ultimately, it may be possible to extend this further, via light-induced RBC docking, drug unloading, and RBC detachment. In short, the technology is extremely flexible since it offers the possibility of separately controlling multiple biological events, including alterations in drug diffusiveness, release of peptides/drugs from the surface of cells, and cell attachment/detachment.

In this study, a new strategy is developed for the creation of photo-activatable agents that represents a marked departure from the approach in place since 1978. These agents not only operate within the optical window of tissue, but they can also be encoded to respond to specific wavelengths. A series of wavelength-triggered anti-inflammatory drug/cobalamin conjugates is prepared. The latter will be delivered using RBCs, the “champions [of] drug delivery systems”, which is now feasible due to the availability of long wavelength-responsive constructs. In addition, proteolytically susceptible peptides can be “hidden” in the plasma membrane protein sheath of RBCs are demonstrated, offering a potentially general strategy for delivering peptides as therapeutic agents. Finally, the therapeutic utility of spatiotemporally controlled drug delivery is investigated using a multiple human cell line-based 3D model system designed to mimic the arthritic arterial/synovium interface.

Next, the synthesis process and characterization of certain exemplary near IR medicated release of anti-inflammatory agents from erythrocyte membranes for highly selective drug delivery is described.

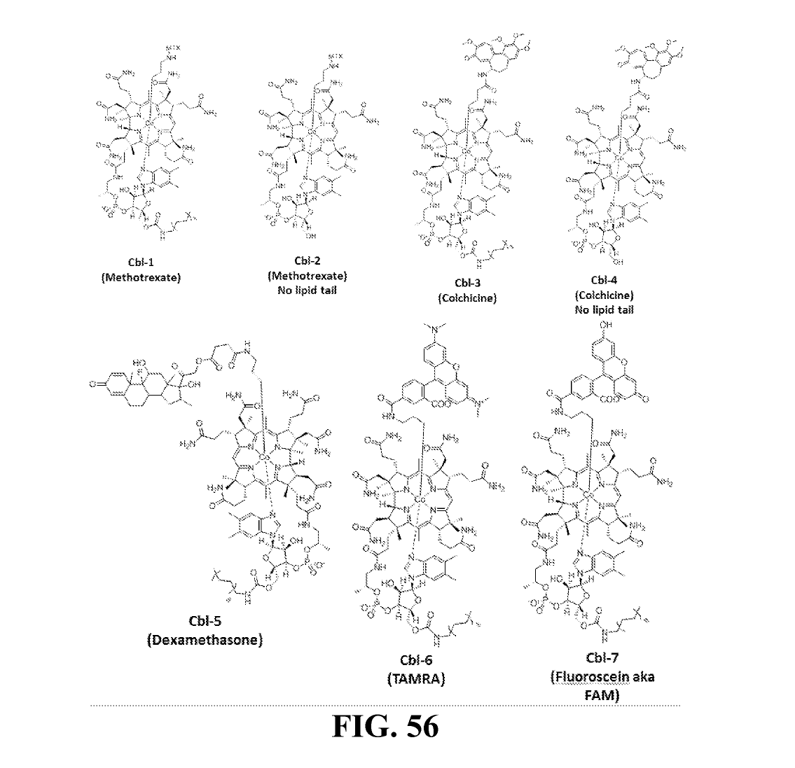

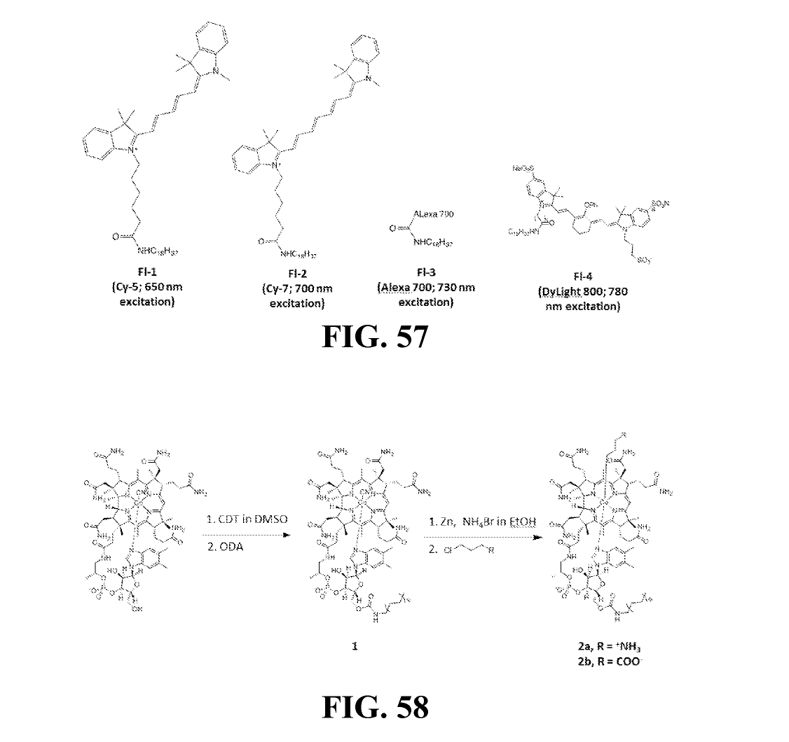

FIG. 56 shows the structures of drug/fluorophore B12 conjugates, including Cbl-1 (methotrexate), Cbl-2 (Methotrexate) without lipid tail, Cbl-3 (Colchicine), Cbl-4 (Colchicine) without lipid tail, Cbl-5 (Dexamethasone), Cbl-6 (TAMRA), Cbl-7 (Fluoroscein aka FAM). FIG. 57 includes structures of flurorophore antennas.

Synthesis of Membrane Anchors 1 (FIG. 58): Octadecylaminylcyanocobalamin (1): Cyanocobalamin (200 mg, 148 μmol, mw=1355) is dissolved in 10 mL of anhydrous DMSO, and CDT (121 mg, 740 μmol, mw=164) is added. The solution is stirred for 45 min. To this solution octadecylamine (398 mg, 1.48 mmol, mw=269) is added to the rapidly stirring solution. The resulting mixture is stirred for 1 hour before being added to 90 mL either/chloroform. The resulting precipitate is collected by centrifugation and decantation. The pellet is dried under vacuum, and 10 mL EtOH is added. Dimerized octadecylamine forms a white precipitate, which is removed by centrifugation, and the cobalamin is precipitated in 40 mL ether/chloroform and collected by centriguation and decantation. The pellet is dissolved in EtOH and purified on a 100 g C18 flash column with a linear gradient an H₂O:MeOH gradient from 0-100% in 8 column volumes. C₁₈ modified cobalamin is eluted at 100% MeOH, with a yield of 75%. (Grissom, C; Lee, M. Org. Lett. 2009, 11, 2499-2502)

Octadecylaminylcobalamin (1): red solid, 75%, ESI MS calculated for C82H125CoN15O15P-(M2+)=825.98. found. (Grissom, C; Lee, M. Org. Lett. 2009, 11, 2499-2502)

Synthesis of Membrane Anchors: 2a (FIG. 58): 3-aminopropyloctadecylaminylcobalamin(2a): 1 (100 mg, 61 μmol, mw=1651) is dissolved in 10 mL of EtOH and degassed under N₂. NH₄Br (500 mg, 5% w/v) and Zn powder (200 mg, 3 mmol) is added, and the solution is stirred for 20 min under N₂. To this slurry 3-chloropropylamine hydrochloride (40 mg, 305 μmol, mw=130) is added. The resulting mixture is stirred for 3 hours under continuous N₂ flow. A color change from red to orange is observed. Zinc is removed by centrifugation, and the cobalamin is recrystallized twice in ether:chloroform (50 mL). The resulting precipitate is collected by centrifugation and decantation. The pellet is dried under vacuum, and 10 mL EtOH is added. UV-Vis analysis reveals that the alkylation went to completion. 2a is purified on a 100 g C₁₈ flash column with a linear gradient an H₂O:MeOH (0.1% TFA) gradient from 0-100% in 8 column volumes. 2a elutes at 100% MeOH.

3-aminopropyloctadecylaminocobalamin (2a): orange solid, [yield question], ESI MS calculated for C₈₄H₁₃₃CoN₁₅O₁₅P (M²⁺)=842.96 is found.

Synthesis of Membrane Anchors: 2b (FIG. 58): Octadecylaminocobalaminbutyrate(2b): 1 (100 mg, 61 μmol, mw=1651) is dissolved in 10 mL of EtOH and degassed under N₂. NH₄Br (500 mg, 5% w/v) and Zn powder (200 mg, 3 mmol) are added, and the solution is stirred for 20 min under N₂. To this slurry, 4-chlorobutryic acid (30 μL, 305 μmol, mw=122, d=1.24) is added. The resulting mixture is stirred for 3 hours under continuous N₂ flow. A color change from red to orange is observed. Zinc is removed by centrifugation, and the cobalamin is recrystallized twice in ether:chloroform (50 mL). The resulting precipitate i collected by centrifugation and decantation. The pellet is dried under vacuum, and 10 mL EtOH is added. UV-Vis analysis reveals the alkylation went to completion. 2b is purified on a 100 g C₁₈ flash column with a linear gradient an H₂O:MeOH (0.1% TFA) gradient from 0-100% in 8 column volumes. 2b elutes at 100% MeOH.

3-aminopropyloctadecylaminocobalamin (2a): orange solid, ESI MS calculated for C₈₅H₁₃₃CoN₁₄O₁₇P⁻ (M²⁺)=855.95 is found.

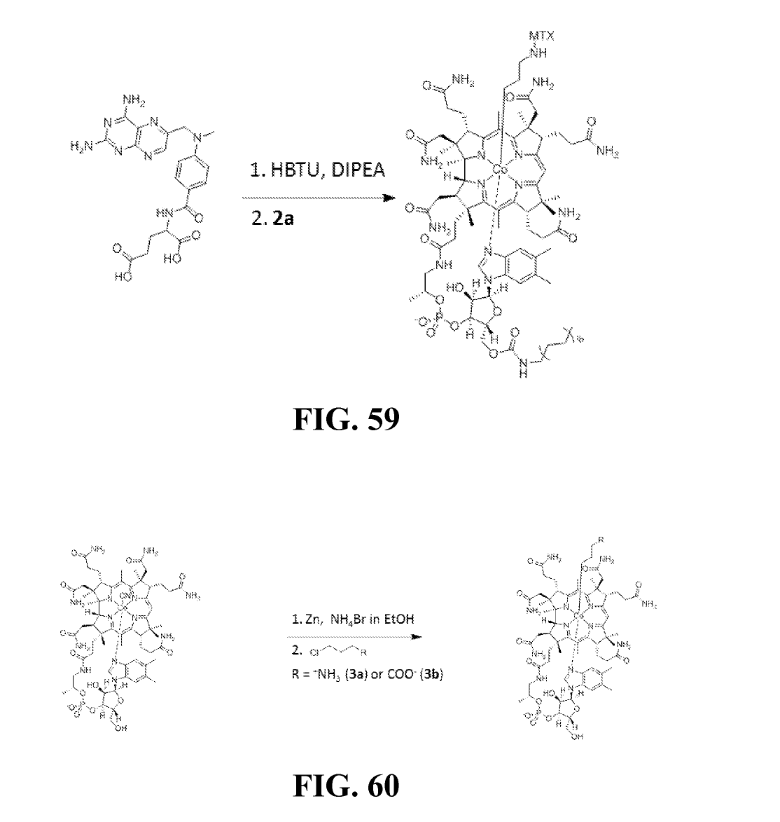

Synthesis of MTX-C₁₈—B₁₂ (FIG. 59): Methotrexate Octadecylaminylcobalamin (cbl-1):

Methotrexate (30 mg, 66 μmol, mw=454), N,N,N; N′-tetramethyl-O-(1H-benzotriazol-1-yl)uronium hexafluorophosphate (HBTU, 25 mg, 66 μmol, mw=379), and N,N-diisopropylethylamine (DIPEA, 58 μL, 332 μmol, mw=129, d=0.74) are dissolved in 5 mL of DMF and stirred for 5 min. 2a (120 mg, 71 μmol, mw=1681) is added, and the solution is stirred overnight. Because 2a and Cbl-1 cannot be separated by HPLC, 1,4,5,6,7,7-hexachloro-5-norbornene-2,3 dicarboxylic anhydride (37 mg, 185 umol, mw=370) is added.

The solution is stirred for 30 minutes and then frozen at −80° C. being careful not to thaw until just before purification. Cbl-1 is purified on a Viva C4 preparative column 5 μm, 250×21.2 mm from Restek, H₂O:ACN, 0.1% TFA, elution time 46 min.

Methotrexate Octadecylaminylcyanocobalamin (Cbl-1): orange solid, 37%, ESI MS calculated for C₁₀₄H₁₅₃CoN₂₃O₁₉P⁻ (M²⁺)=1059.7. found 1060.3; (M³⁺)=706.5. found 707.2.

Synthesis of Monofunctionalized Cobalamin: 3a (FIG. 60): 3-aminopropylcobalamin (3a): Cyanocobalamin (200 mg, 148 μmol, mw=1355) is dissolved in 10 mL of MeOH and degassed under N₂. NH₄Br (500 mg, 5% w/v) and Zn powder (200 mg, 3 mmol) are added, and the solution is stirred for 20 minutes under N₂. To this slurry, 3-chloropropylamine hydrochloride (40 mg, 305 μmol, mw=130) is added. The resulting mixture is stirred for 3 h under continuous N₂ flow. A color change from red to orange is observed. Zinc is removed by centrifugation, and the cobalamin is recrystallized twice in ether:chloroform (50 mL). The resulting precipitate was collected by centrifugation and decantation. The pellet is dried under vacuum, and 10 mL EtOH is added. UV-Vis analysis reveals that the alkylation went to completion. 3a is purified on a 100 g C₁₈ flash column with a linear gradient and H₂O:MeOH (0.1% TFA) gradient from 0-100% in 8 column volumes. 3a elutes at 50% MeOH.

Synthesis of Monofunctionalized Cobalamin: 3 b (FIG. 60): cobalaminbutyrate(3b): Cyanocobalamin (200 mg, 148 μmol, mw=1355) is dissolved in 10 mL of MeOH and degassed under N₂. NH₄Br (500 mg, 5% w/v) and Zn powder (200 mg, 3 mmol) are added, and the solution is stirred for 20 minutes under N₂. To this slurry, 3-chloropropylamine hydrochloride (40 mg, 305 μmol, mw=130) is added. The resulting mixture is stirred for 3 hours under continuous N₂ flow. A color change from red to orange is observed. Zinc is removed by centrifugation, and the cobalamin is recrystallized twice in ether:chloroform (50 mL). The resulting precipitate is collected by centrifugation and decantation. The pellet is dried under vacuum, and 10 mL EtOH is added. UV-Vis analysis reveals the alkylation went to completion. 3b is purified on a 100 g C₁₈ flash column with a linear gradient and H₂O:MeOH (0.1% TFA) gradient from 0-100% in 8 column volumes. 3b elutes at 60% MeOH.

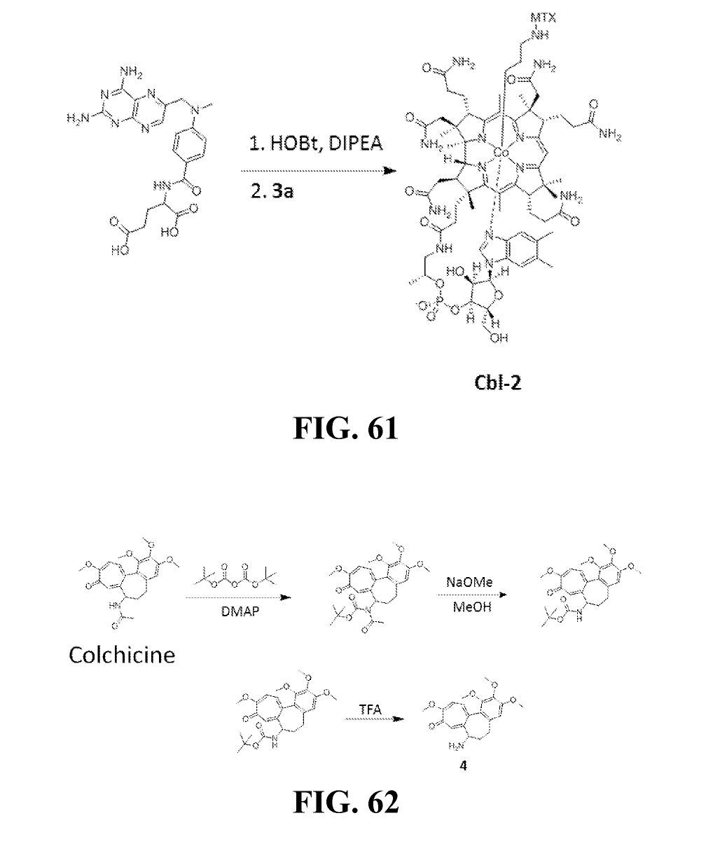

Synthesis of MTX-B₁₂ (Cbl-2): (FIG. 61): Methotrexate cobalamin (Cbl-2): Methotrexate (30 mg, 66 μmol, mw=454), N,N,N′,N′-tetramethyl-O-(1H-benzotriazol-1-yl)uronium hexafluorophosphate (HBTU, 25 mg, 66 μmol, mw=379), and N,N-diisopropylethylamine (DIPEA, 58 μL, 332 μmol, mw=129, d=0.74) are dissolved in 5 mL of DMF and stirred for 5 min. 3a (98 mg, 71 μmol, mw=1386) is added, and the solution is stirred overnight. Cbl-2 is purified on a 100 g C₁₈ flash column with a linear gradient an H₂O:MeOH (0.1% TFA) gradient from 0-100% in 8 column volumes.

Methotrexate cobalamin (Cbl-2): orange solid, 65%, ESI MS calculated for C₈₆H₁₁₇CoN₂₁O₁₈P⁻ (M²⁺)=910.9. found 912.6; (M³⁺)=607.2. found 608.8.

Synthesis of Deacetylcolchicine is shown in FIG. 62 as previously described. (Lebeau, L.; Ducray, P.; Mioskowski, C. SYNTH. COMMUN. 1997, 27, 293-296.)

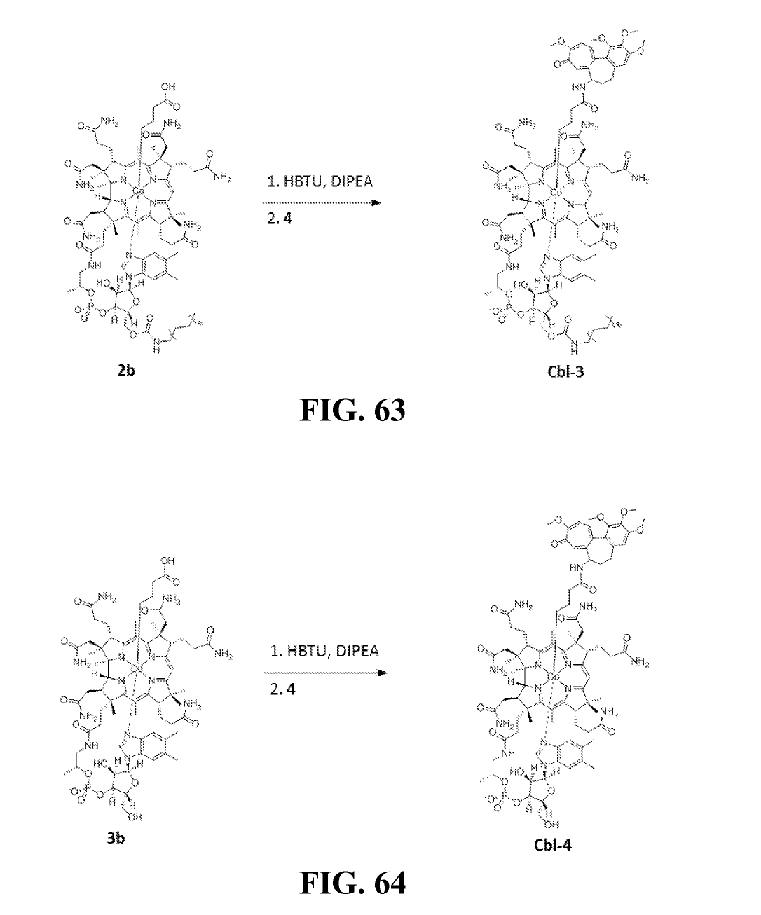

Synthesis of Colchicine-C₁₈—B₁₂ (Cbl-3) is shown in FIG. 63. Colchicine octadecylaminylcobalamin (Cbl-3): 2b (63 mg, 37 μmol, mw=1681), N,N,N′,N′-tetramethyl-O-(1H-benzotriazol-1-yl)uronium hexafluorophosphate (HBTU, 10 mg, 26 μmol, mw=379), and N,N-diisopropylethylamine (DIPEA, 15 μL, 86 μmol, mw=129, d=0.74) are dissolved in 2 mL of DMF and stirred for 5 min. 4 (10 mg, 28 μM, mw=1386) is added, and the solution is stirred overnight. Cbl-3 is purified on a Viva C4 preparative column 5 μM, 250×21.2 mm) from Restek, H₂O:CH₃CN, 0.1% TFA, elution time 35 min.

Colchicine octadecylaminylcobalamin (Cbl-3): orange solid, ESI MS calculated for C₁₀₅H₁₅₃CoN₁₅O₂₁P⁻ (M²⁺)=1025.0. found 1026.5; (M³⁺)=683.3. found 684.5.

Synthesis of Colchicine-B₁₂ (Cbl-4) is shown in FIG. 64: Colchicine cobalamin (Cbl-4): 3b (58 mg, 41 μmol, mw=1416), N,N,N,′N′-tetramethyl-O-(1H-benzotriazol-1-yl)uronium hexafluorophosphate (HBTU, 10 mg, 26 μmol, mw=379), and N,N-diisopropylethylamine (DIPEA, 15 μL, 86 μmol, mw=129, d=0.74) are dissolved in 2 mL of DMF and stirred for 5 minutes. 4 (10 mg, 28 μmol, mw=1386) is added, and the solution is stirred overnight. Cbl-4 is purified on a 100 g C₁₈ flash column with a linear gradient an H₂O:MeOH (0.1% TFA) gradient from 0-100% in 8 column volumes.

Colchicine cobalamin (Cbl-4): orange solid, ESI MS calculated for C₈₆H₁₁₆CoN₁₄O₂₀P⁻ (M²⁺)=877.4. found 878.5; (M³⁺)=584.9. found 586.2.

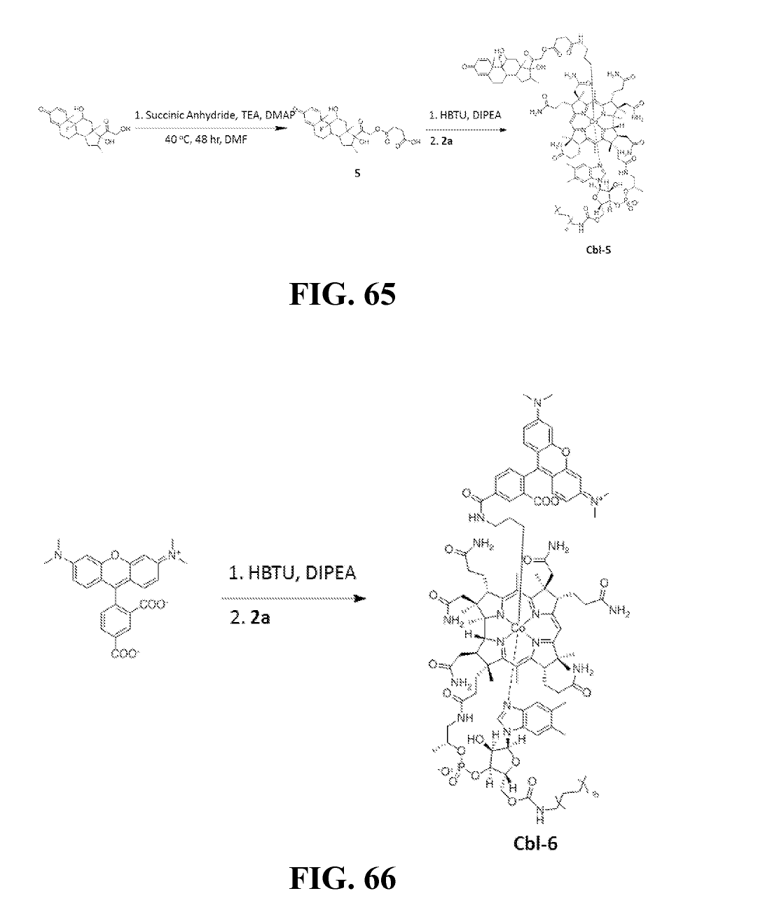

Synthesis of DEX-C₁₈—B₁₂ (Cbl-5) is shown in FIG. 65. Dexamethasone succinyloctadecylaminylcobalamin (Cbl-5): 5 (6 mg, 12 μmol, mw=492), N,N,N′,N′-tetramethyl-O-(1H-benzotriazol-1-yl)uronium hexafluorophosphate (HBTU, 5 mg, 12 μmol, mw=379), and N,N-diisopropylethylamine (TEA, 10 μL, 57 μmol, mw=129, d=0.74) are dissolved in 1 mL of DMF and stirred for 5 minutes. 2a (30 mg, 18 μmol, mw=1681) is added, and the solution is stirred overnight. Cbl-5 is purified on a Viva C4 preparative column 5 μm, 250×21.2 mm) from Restek, H₂O:CH₃CN, 0.1% TFA, elution time 62 min.

Dexamethasone succinyloctadecylaminylcobalamin (Cbl-5): orange solid, ESI MS calculated for C₁₁₀H₁₆₄CoN₁₅O₂₂P⁻ (M²⁺)=1078.1. found 1079.3.

Synthesis of 5-TAM-C₁₈—B₁₂ (Cbl-6) is shown in FIG. 66. 5-TAMRA Octadecylaminylcobalamin (Cbl-6): 5-TAMRA (5 mg, 12 μmol, mw=430), N,N,N′,N′-tetramethyl-O-(1H-benzotriazol-1-yl)uronium hexafluorophosphate (HBTU, 4.5 mg, 12 μmol, mw=379), and N,N-diisopropylethylamine (DIPEA, 8.3 μL, 48 μmol, mw=129, d=0.74) are dissolved in 5 mL of DMF and stirred for 5 minutes. 2a (20 mg, 12 μmol, mw=1681) is added, and the solution is stirred overnight. Cbl-6 is purified on a Viva C₄ preparative column 5 μm, 250×21.2 mm) from Restek, H₂O:CH₃CN, 0.1% TFA, elution time 46 min.

5-TAMRA Octadecylaminylcobalamin (Cbl-6): red solid, ESI MS calculated for C₁₀₉H₁₅₄CoN₁₇O₁₉P⁻ (M²⁺)=1047.5. found 1048.7; (M³⁺)=698.3. found 699.3.

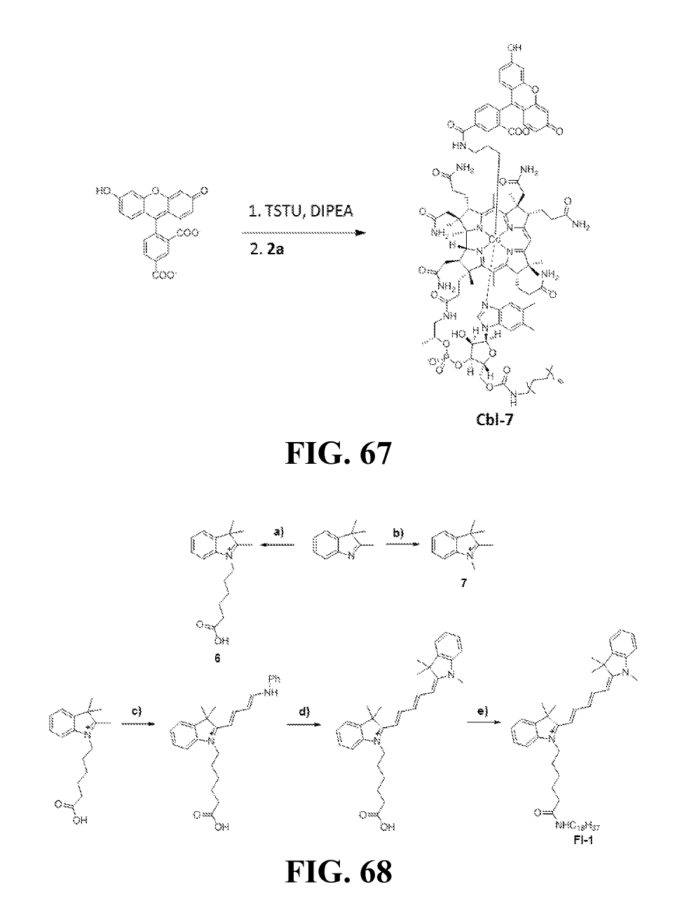

Synthesis of 5-FAM-C₁₈—B₁₂ (Cbl-7) is shown in FIG. 67. 5-FAM Octadecylaminylcobalamin (Cbl-7): 5-FAM (5 mg, 12 μmol, mw=430), N,N,N′N′-tetramethyl-O—(N-succinimidyl)uronium tetrafluoroborate (TSTU, 3.6 mg, 12 μmol, mw=301), and N,N-diisopropylethylamine (DIPEA, 8.3 μL, 48 μmol, mw=129, d=0.74) are dissolved in 5 mL of DMF and stirred for 5 minutes. 2a (20 mg, 12 μmol, mw=1681) is added, and the solution is stirred overnight. Cbl-7 is purified on a Viva C₄ preparative column 5 μm, 250×21.2 mm) from Restek, H₂O:CH₃CN, 0.1% TFA, elution time 46 min.

5-FAM-octadecylaminylcobalamin (Cbl-7): orange solid, ESI MS calculated for C₁₀₄H₁₄₃CoN₁₅O₁₉P⁻ (M²⁺)=1020.0. found 1021.5; (M³⁺)=680.0. found 681.2.

Synthesis of Cy5-C₁₈(Fl-1) is shown in FIG. 68. Synthesis of Cy5-C18 (4) a) Br(CH₂)₅CO₂H, KI, CH₃CN b) CH₃I c) malonaldehyde dianilide, AcOH, Ac₂O d) 2, pyridine, AcOH e) DIC (N,N′-diisopropylcarbodiimide), TEA, octadecylamine, CH₂Cl₂. Cy5 is synthesized as previously reported (Kiyose, K.; Hanaoka, K.; Oushiki, D; Nakamura, T.; Kajimura, M.; Suematsu, M.; Nishimatsu, H.; Yamane, T.; Terai, T; Hirata, Y; and Nagano, T. JACS. 2010, 132, 15846-15848.)

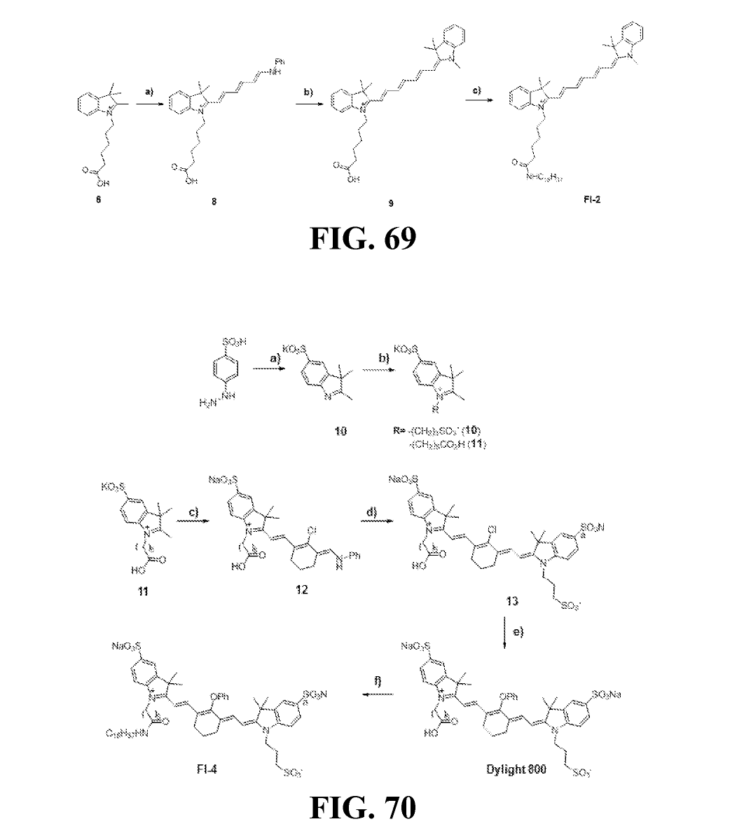

Synthesis of Cy7-C₁₈ (Fl-2) is shown in FIG. 69. Synthesis of Cy7-C18 (6) a)N-[5-(Phenylamino)-2,4-pentadienylidene]aniline monohydrochloride, AcOH, Ac₂O b) 7, AcOH, pyridine c) DIC, TEA, octadecylamine, CH₂Cl₂. (Kiyose, K.; Hanaoka, K.; Oushiki, D; Nakamura, T.; Kajimura, M.; Suematsu, M.; Nishimatsu, H.; Yamane, T.; Terai, T; Hirata, Y; and Nagano, T. JACS. 2010, 132, 15846-15848.)

Synthesis of Alexa-700-C₁₈ (Fl-3). Fl-3: Alexa Fluor® 700 NHS-ester (1 mg, 1 μmol, mw=1086), N,N-diisopropylethylamine (DIPEA, 5 μL, 29 μmol, mw=129, d=0.74), and octadecylamine (5 mg, 19 μmol, mw=269) are dissolved in 500 μL DMF and mixed by agitation overnight. The resulting mixture is poured into 5:1 H₂O:CH₂Cl₂ mixture (5 mL). The CH₂Cl₂ layer is washed (3×) with 4 mL H₂O. Purified by flash chromatography silica column (30 g). MeOH:CH₂Cl₂ (0.1% TFA) linear gradient 0-80%. The purified lipidated fluorophore is concentrated by rotary evaporation. (Note: the structure of Alexa Fluor®700 has not been disclosed. Therefore just the experimental is furnished.)

Synthesis of Dy800-C₁₈ (Fl-4) is shown in FIG. 70. Synthesis of Dy800-C₁₈ (12) a) 3-methyl butanone, AcOH; KOH, MeOH, PrOH b) (10): 1,3-propane sultone, o-dichlorobenzene (11): Br(CH₂)₅CO₂H, o-dichlorobenzene c) 3-chloro-2,4-trimethyleneglutacondianil hydrochloride, AcONa, EtOH d) 10 e) sodium phenoxide, DMF f) DIC, DIPEA, octadecylamine, DMF

Table 13 Provides the HPLC Gradient for Column C₄

| Time | Flow (mL/min) | Water | CH₃CN |
|---|---|---|---|
| 0 | 8 | 90 | 10 |
| 10 | 8 | 90 | 10 |
| 30 | 8 | 45 | 55 |
| 45 | 8 | 35 | 65 |
| 60 | 8 | 25 | 75 |
| 65 | 8 | 0 | 100 |
| 75 | 8 | 0 | 100 |
| 80 | 8 | 90 | 10 |
| 90 | 8 | 90 | 10 |

Further, these examples describe that fluorophores are released from RBC membranes.

Demonstrating TAMRA and fluorescein (FAM) release from erythrocyte membranes using 525 nm light: Erythrocytes are washed 3× in 1×PBS containing 1 mM MgCl₂ and diluted to 10% hematocrit. To 10% hematocrit erythrocytes, Cbl-6 (releases TAMRA) or Cbl-7 (releases fluoroscein) is added to a final concentration of 1 μM. The erythrocytes are then incubated at RT for 20 min and subsequently washed 3× in 1×PBS containing 1 mM MgCl₂. After the final wash, erythrocytes are resuspended to 10% hematocrit and exposed to 525 nm light for various time points. After photolysis, the erythrocyte solution is centrifuged at 1,000 g, and the supernatant is analyzed for TAMRA (Ex: 550 nm Em: 580 nm) or Fluorescein (Ex: 492 nm Em: 519 nm) release using a fluorescent plate reader.

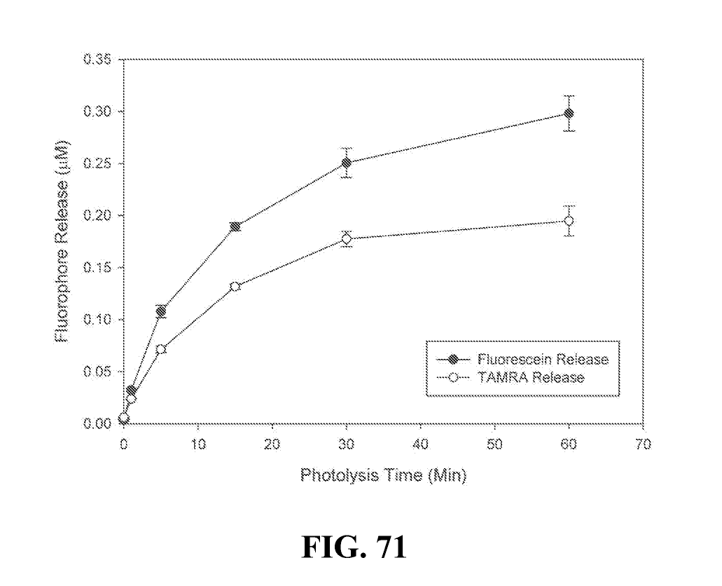

FIG. 71 shows Cbl-6 and Cbl-7 Photocleaved from RBC Membranes. Fluorescein release and TAMRA release from cobalamins (Cbl-7 and Cbl-6, respectively) bound to erythrocytes using 525 nm light.

Demonstrating TAMRA (from Cbl-6) and Fluorescein (from Cbl-7) release from erythrocyte membranes using NIR light. Erythrocytes are washed 3× in 1×PBS containing 1 mM MgCl₂ and diluted to 10% hematocrit. To 10% hematocrit erythrocytes, Cbl-6 or Cbl-7 is added to a final concentration of 1 μM and either Fl-1, Fl-2, Fl-3, or Fl-4 to a final concentration of 5 μM. The erythrocytes are then incubated at RT for 20 min and subsequently washed 3× in 1×PBS containing 1 mM MgCl₂. After the final wash erythrocytes are resuspended to 10% hematocrit and exposed to 650, 700, 730, or 780 nm light for 30 min. After photolysis, the erythrocyte solution is centrifuged at 1,000 g, and the supernanent is analyzed for TAMRA (Ex: 550 nm Em: 580 nm) or Fluorescein (Ex: 492 nm Em: 519 nm) release using a fluorescent plate reader.

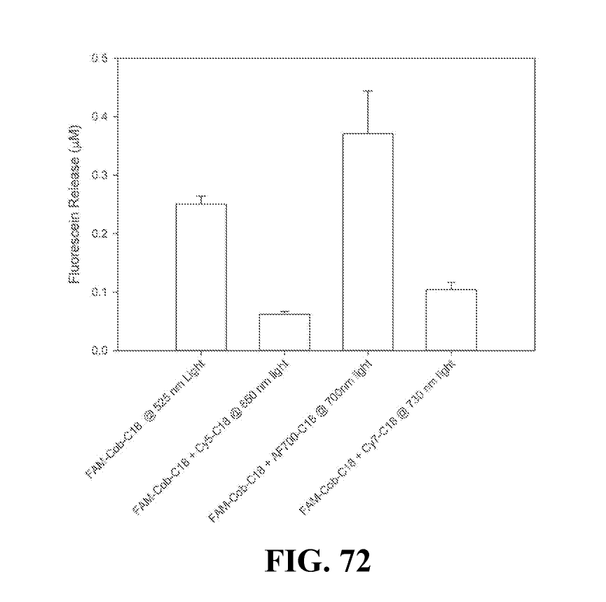

FIG. 72 shows using C₁₈ Conjugated Fluorophores to Extend Photocleavage of FAM into the Near IR (NIR). Releasing Fluorescein (from Cbl-7) using Fl-1 (650 nm), Fl-2 (700 nm), and Fl-3 (730 nm). Erythrocytes are loaded with 1 μM Cbl-7 and 5 μM Fluorophore-C₁₈. Photolysis is performed using the above mentioned wavelengths of light for 30 min. NOTE: Cobalamin (aka B₁₂) only absorbs light up to around 550 nm; therefore in order to absorb light beyond this wavelength, the presence of an antenna fluorophore is required.

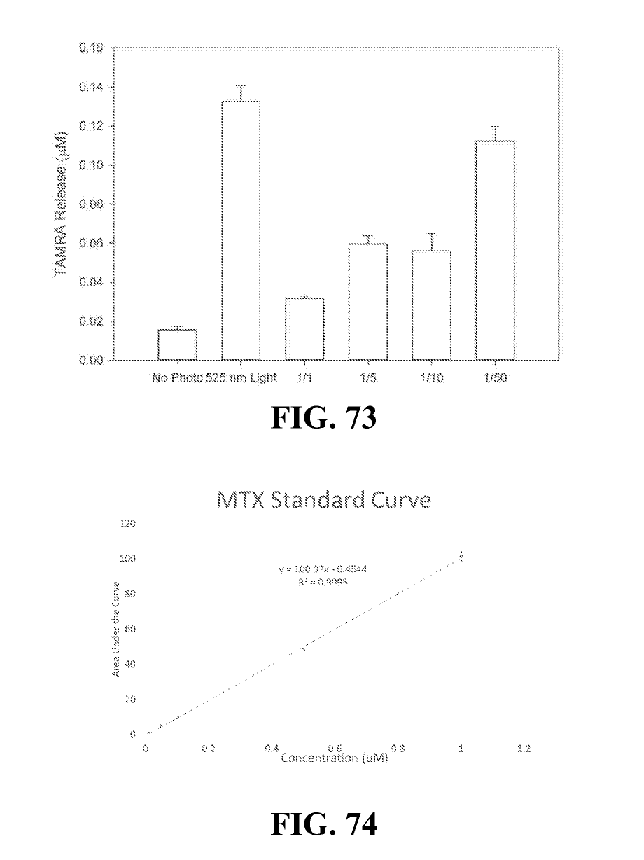

Determining the Ratio of Cbl-6 to Fl-1 for optimal release of TAMRA. Erythrocytes are washed 3× in 1×PBS containing 1 mM MgCl₂ and diluted to 10% hematocrit. To 10% hematocrit erythrocytes, Cbl-6 is added to a final concentration of 1 μM and Fl-1 to a final concentration of 0, 1, 5, 10, and 50 μM. The erythrocytes are then incubated at RT for 20 minutes and subsequently washed 3× in 1×PBS containing 1 mM MgCl₂. After the final wash, erythrocytes are resuspended to 10% hematocrit and exposed to 650 nm light for 30 minutes. After photolysis, the erythrocyte solution is centrifuged at 1,000 g, and the supernanent is analyzed for TAMRA (Ex: 550 nm Em: 580 nm) release using a fluorescent plate reader. FIG. 73 demonstrates determining [Cbl-6]:[Fl-1] the Ratio of Optimal Release Using 650 nm Light.

This further example describes the photo release of MTX erythrocyte membranes.

Table 14 shows the determination of MTX concentration by LC-MS. Samples of 75 μL, are injected onto an 1200 series Agilent HPLC with a UV-Vis detector, 1260 infinity fluorescent detector, and 6110 quadrapole mass spectrometer from a 394 well plate. The mobile phase consists of H₂O:CH₃CN (0.1% FA) (gradient provided in the following Table 14). The column used is a Viva C₄ analytical column 5 μm, 50×21.2 mm from Restek. Concentrations are determined by taking the area under UV absorbance trace at 300 nm from 3.1-3.7 min where the MTX cleavage products are shown to elute, and this integration is compared to known standards. Mass cutoff is 450 daltons. Fluorescent detector ex. 365 nm em. 470 nm to detect known products of light degradation.

TABLE 14

| Time | Flow (mL/min) | Water | CH3CN |
|---|---|---|---|
| 0 | 1 | 97 | 3 |
| 1 | 1 | 97 | 3 |
| 8 | 1 | 20 | 80 |
| 10 | 1 | 20 | 80 |
| 11 | 1 | 97 | 3 |
| 15 | 1 | 97 | 3 |

FIG. 74 shows an MTX standard curve. Cbl-1 dilutions are prepared at 1 μM, 500 nM, 100 nM, 50 nM, and 10 nM concentrations. These are photolyzed under 525 nm light until no intact Cbl-1 is detected. Then 100 uL aliquots are taken for analysis by LC-MS, and the area under the curve is calculated for each concentration. This is done in triplicate, and all [MTX] data is generated by comparisons to the resulting standard curve.

Photo release of methotrexate (MTX) from erythrocyte membranes. Erythrocytes are washed 3× in 1×PBS containing 1 mM MgCl₂ and diluted to 10% hematocrit. To 10% hematocrit erythrocytes, Cbl-1 is added to a final concentration of 1 μM and/or 5 μM Fl-1. The erythrocytes are then incubated at RT for 20 min and subsequently washed 3× in 1×PBS containing 1 mM MgCl₂. After the final wash, erythrocytes are resuspended to 10% hematocrit and exposed to 525 or 650 nm light for 10, 30, and 60 min. After photolysis, the erythrocyte solution is centrifuged at 1,000 g, and the supernatant is analyzed for MTX release by LC/MS.

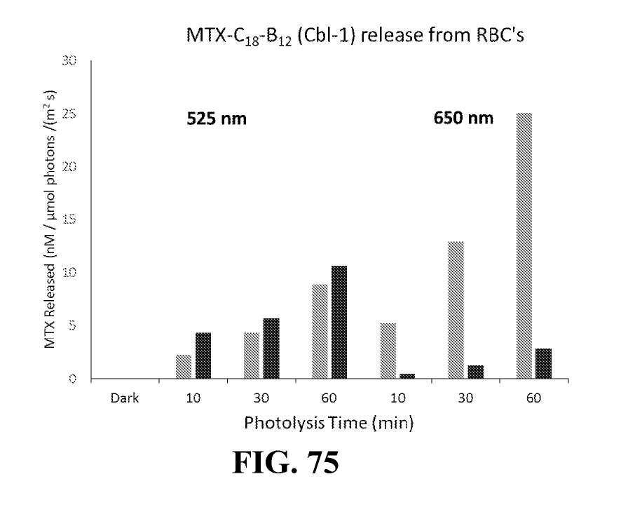

FIG. 75 demonstrates MTX-C18-B12 (CBl-1) releasing from RBCs. Releasing MTX from RBCs over time using 525 nm light and 650 nm light. The orange indicates the presence of 5 μM Fl-1 and 1 μM Cbl-1. The blue samples contain only Cbl-1. Fl-1 is thus required for efficient drug release at 650 nm.

Methotrexate DHFR inhibition assay. Dihydrofolate reductase activity is monitored using the Sigma Dihydrofolate Reductase Assay Kit. This kit is used to monitor conversion of NADPH to NADP⁺. Briefly, assay buffer is prepared containing 1.5 mU DHFR, 100 μM NADPH, and 1× assay buffer (provided with kit). Inhibition of DHFR activity at various concentrations MTX or Photolyzed MTX from MTX-C₁₈—B₁₂ (between 100 nm and 5 μM) is monitored using a fluorescent plate reader (Ex: 340 nm Em: 450 nm).

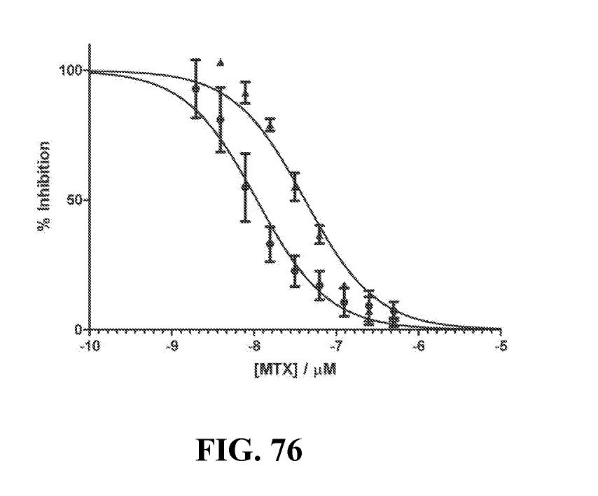

FIG. 76 demonstrates DHFR inhibition assay of MTX. DHFR is inhibited by methotrexate (circles) and photolyzed methotrexate (triangles).

Determination of colchicine concentration by LC-MS (Table 15). Samples of 75 μL, are injected onto an 1200 series Agilent HPLC with a UV-Vis detector, 1260 infinity fluorescent detector and 6110 quadrapole mass spectrometer from a 394 well plate. The mobile phase consists of H₂O:CH₃CN (0.1% FA) (gradient provided in the following table). The column used is a Viva C₄ analytical column 5 μm, 50×21.2 mm from Restek. Concentrations are determined by taking the area under UV absorbance trace at 360 nm from 4.1-4 8 min where the colchicine cleavage products were shown to elute and this integration was compared to known standards. Mass cutoff is 400 daltons.

TABLE 15

| Time | Flow (mL/min) | Water | CH₃CN |
|---|---|---|---|
| 0 | 1 | 97 | 3 |
| 1 | 1 | 97 | 3 |
| 8 | 1 | 20 | 80 |
| 10 | 1 | 20 | 80 |
| 11 | 1 | 97 | 3 |
| 15 | 1 | 97 | 3 |

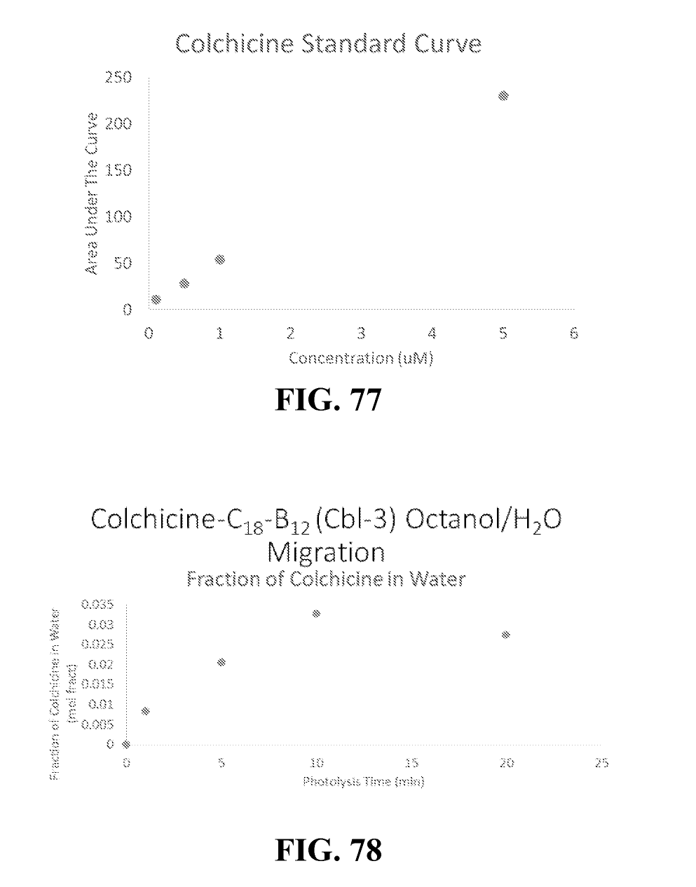

Colchicine standard curve. Cbl-3 dilutions are prepared at 5 μM, 1 μM, 500 nM, and 100 nM concentrations in 10% allyl alcohol and water. These are photolyzed under 525 nm light until no intact Cbl-3 is detected. Then 100 μL aliquots are taken for analysis by LC-MS, and the area under the curve is calculated for each concentration. This is done in triplicate, and all subsequent colchicine concentration data is generated by comparisons to the resulting standard curve. FIG. 77 shows the colchicine standard curve.

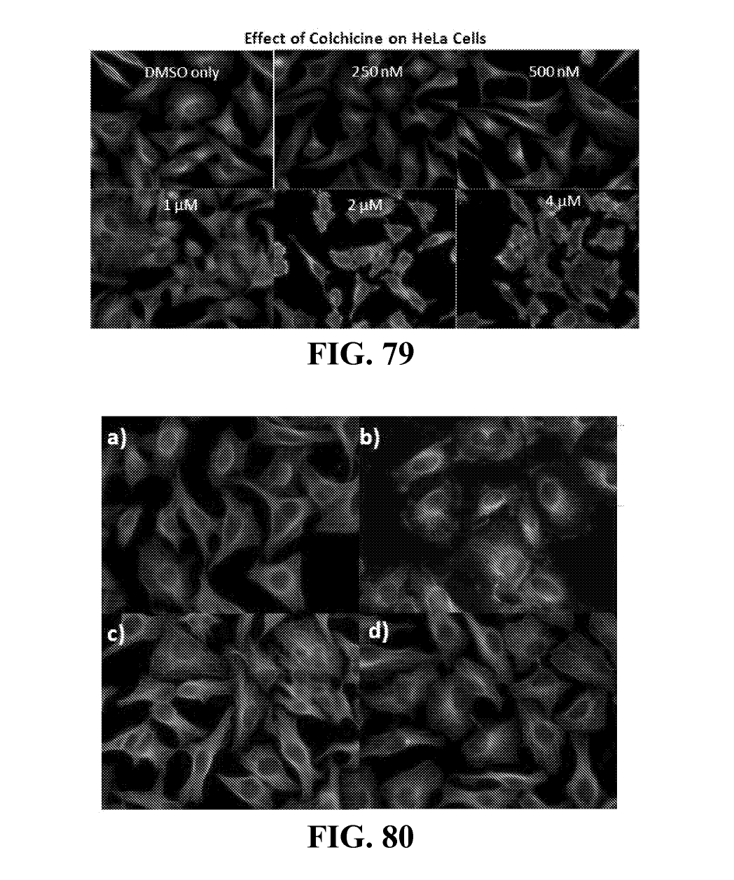

FIG. 78 shows colchicine-C₁₈—B₁₂ (Cbl-3) Octanol/H₂O Migration. Photolyzed colchicine (from Cbl-3) diffuses from octanol into water and does so in increasing amounts until maximal photolysis at 10 min. Due to the hydrophobic nature of the molecule, the equilibrium prefers octanol even after cleavage, but there is no detectable migration into the water until cleavage occurs.

Treatment of HeLa cells with C₁₈—B₁₂-methotrexate loaded RBCs. HeLa cells are plated in 12 well tissue culture plates at a density of 4.4×10⁴ cells per well and maintained at 37° C. in a humidity-controlled incubator with a 5% CO₂ atmosphere in DMEM (10% FBS, 1% Pen-Strep). The following day, cells are washed 2× with PBS, then treated with 300 μL of a suspension of Cbl-1 loaded RBCs in L-15 media (5 μM loading volume at 5% hemocrit) or 300 μL, L-15 (control cells). The cells are either kept in the dark or exposed to a green LED light source (PAR38; 500-570 nm emission; 5 mW power) for 15 minutes. After addition of a small aliquot of FBS-containing media to bring the serum concentration to 0.5%, cells are subsequently placed in a 37° C. in a humidity-controlled incubator. After 48 hours, cells are washed 3×1 mL with PBS, and 400 uL of L-15 media is added to each well, followed by the addition of 80 μL of MTS reagent (Promega CellTiter 96 Aqueous One Solution). Cells are incubated with the MTS reagent for 3 h at 37° C., and the absorbance at 492 nm is measured with a plate reader (Perkin Elmer HTS 7000).

Partition study. Octanol (250 μL) containing the study molecule (5 μM) is thoroughly mixed with dH₂O (250 μL) in a 1.5 mL clear centrifuge tube and allowed to equilibrate for 10 minutes before undergoing centrifugation for 10 minutes at 21,000 g. Samples are photolyzed with a 525 nm LED for 0, 1, 5, 10, and 20 minutes before being mixed by shaking and allowed to equilibrate for 15 minutes. This is followed by a 10 minute centrifugation at 21,000 g. Aliquots are taken from the desired layer(s), and the concentrations of each are determined by LC-MS methods specific to the chemical in question.

Treatment of HeLa cells with colchicine. HeLa cells are plated in a 6-well glass bottom plate (Mattek) at a density of 1.5×10⁵ cells per well and maintained at 37° C. in a humidity-controlled incubator with a 5% CO₂ atmosphere in DMEM (10% FBS, 1% Pen-Strep). The following day, cells are treated with colchicine (Sigma C9754; 1 mM stock in DMSO) or DMSO for either 30 minutes or 1 hour at 37° C. in a humidity-controlled incubator. At the conclusion of the incubation period, cells are fixed with 1 mL of methanol at room temperature for 10 minutes. Cells are washed 2×1 mL with PBS and blocked for 1 h in 5% donkey serum. Blocking is followed by overnight incubation at 4° C. with mouse anti-tubulin antibody (Cell Signaling 3873S) at 1:100 dilution in antibody dilution buffer (1% BSA; 0.3% Triton-X-100; PBS). Cells are then washed with PBS (3×5 min) before incubation with anti-mouse Alexa Fluor® 488 secondary antibody (Life Technologies A21202) at 1:500 dilution in antibody dilution buffer. After washing cells with PBS (3×5 min), images are acquired with an inverted Olympus IX81 microscope equipped with a Hamamatsu C8484 camera, 40× phase contrast objective, and a FITC filter cube (Semrock). Metamorph software is employed for imaging analysis.

FIG. 79 shows the effect of colchicine on HeLa cells. This is the positive control. As more colchicine is added, the tubulin networks become disrupted.

Treatment of HeLa cells with Cbl-3 loaded RBCs. HeLa cells are plated in 24-well glass bottom plates (Mattek) at a density of 3.3×10⁴ cells per well and maintained at 37° C. in a humidity-controlled incubator with a 5% CO₂ atmosphere in DMEM (10% FBS, 1% Pen-Strep). The following day, cells are washed twice with PBS, followed by the addition of 100 uL of L-15 media. Cells are then treated with 250 μL of a suspension of Cbl-3 loaded red blood cells in PBS (6 μM loading volume at 5% hemocrit) or 250 μL PBS (control cells). Cells are then either kept in the dark at 37° C. in a humidity-controlled incubator, or exposed to 530 nM LED flood light (PAR38; 500-570 nm emission; 5 mW power) for 5, 10, or 20 min at room temperature. All cells are incubated for 1 hour in a 37° C. humidity-controlled incubator post-photolysis. At the conclusion of the incubation period, cells are washed 3×1 mL with PBS and then fixed with 1 mL of methanol at room temperature for 10 minutes. Cells are washed 2×1 mL with PBS and blocked for 1 h in 5% donkey serum. Blocking is followed by overnight incubation at 4° C. with mouse anti-tubulin antibody (Cell Signaling 3873S) at 1:100 dilution in antibody dilution buffer (1% BSA; 0.3% Triton-X-100; PBS). Cells are then washed with PBS (3×5 min) before incubation with anti-mouse Alexa Fluor® 488 secondary antibody (Life Technologies A21202) at 1:500 dilution in antibody dilution buffer. After washing cells with PBS (3×5 min), images are acquired with an inverted Olympus IX81 microscope equipped with a Hamamatsu C8484 camera, 40× phase contrast objective, and a FITC filter cube (Semrock). Metamorph software is employed for imaging analysis.

FIG. 80 shows the effect of Cbl-3 on HeLa cells. a) HeLa cells exposed to Cbl-3 loaded RBCs without photolysis. b) HeLa cells exposed to Cbl-3 loaded RBCs illuminated with 525 nm light for 20 min. c) HeLa cells with no RBC or light exposure. d) HeLa cells without RBCs and with 20 minute photolysis at 525 nm.

The following example describes the photorelease of dexamethasone. HeLa cells are plated in a 6-well glass bottom plate (Mattek) at a density of 7.5×10⁴ cells per well and maintained at 37° C. in a humidity-controlled incubator with a 5% CO₂ atmosphere in DMEM (10% FBS, 1% Pen-Strep). The following day, cells are treated with varying concentrations of dexamethasone (1 mM stock in DMSO) or DMSO for 1 hour at 37° C. in a humidity-controlled incubator. At the conclusion of the incubation period, cells are fixed with 4% PFA in PBS for 10 minutes at room temperature, then washed 1× with PBS, and then treated with 1 mL of methanol at room temperature for 5 min. Cells are washed 2×1 mL with PBS and subsequently incubated overnight at 4° C. with rabbit anti-GRα antibody (abcam 3580) at 1:100 dilution in antibody dilution buffer (1% BSA; 0.3% Triton-X-100; PBS). Cells are then washed with PBS (3×5 min) before incubation with anti-rabbit Alexa Fluor® 488 secondary antibody (Life Technologies A21206) at 1:500 dilution in antibody dilution buffer for 1 hour at room temperature. Cells are washed with PBS (3×5 min), and Hoescht 33342 (100 μg/mL in PBS) is applied for 30 minutes before an additional wash with PBS. Images are subsequently acquired with an inverted Olympus IX81 microscope equipped with a Hamamatsu C8484 camera, 40× phase contrast objective and a FITC filter cube (Semrock). Metamorph software is employed for imaging analysis.

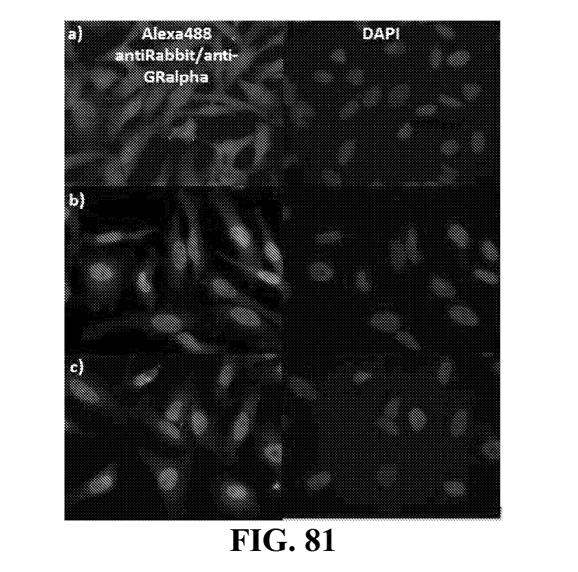

FIG. 81 shows the effects of dexamethasone on the distribution of GRα. The steroid receptor is evenly distributed in the cystosol in a) due to the absence of dexamethasone. After the addition of 250 nM dexamethasone in b) the receptor migrates to the nucleus and the same is observed in c) with 500 nM dexamethasone.

Treatment of HeLa cells with Cbl-5 loaded RBCs. HeLa cells are plated in 12-well glass bottom plates (Mattek) at a density of 2.5×10⁴ cells per well and maintained at 37° C. in a humidity-controlled incubator with a 5% CO₂ atmosphere in DMEM (10% FBS, 1% Pen-Strep). The following day, cells are washed 2× with PBS, then treated with 500 μL of a suspension of Cbl-5 loaded red blood cells in L-15 media (1 μM loading volume at 5% hemocrit) or 500 μL L-15 (control cells). Cells are then either kept in the dark at 37° C. in a humidity-controlled incubator or exposed to 525 nM LED flood light (PAR38; 500-570 nm emission; 5 mW power) for 10, 20, or 30 minutes at room temperature. All cells are incubated for 1 hour in a 37° C. in a humidity-controlled incubator post-photolysis. At the conclusion of the incubation period, cells are washed 3×1 mL with PBS and then fixed with with 4% PFA in PBS for 10 minutes at room temperature, then washed 1× with PBS and treated with 1 mL of methanol at room temperature for 5 minutes. Cells are subsequently washed 2×1 mL with PBS and then incubated overnight at 4° C. with rabbit anti-GRα antibody (abcam 3580) at 1:100 dilution in antibody dilution buffer (1% BSA; 0.3% Triton-X-100; PBS). Next, cells are washed with PBS (3×5 min) before incubation with anti-rabbit AlexaFluor® 488 secondary antibody (Life Technologies A21206) at 1:500 dilution in antibody dilution buffer for 1 hour at room temperature. Cells are finally washed with PBS (3×5 min). Images are subsequently acquired with an inverted Olympus 1×81 microscope equipped with a Hamamatsu C8484 camera, 40× phase contrast objective and a FITC filter cube (Semrock). Metamorph software is employed for imaging analysis.

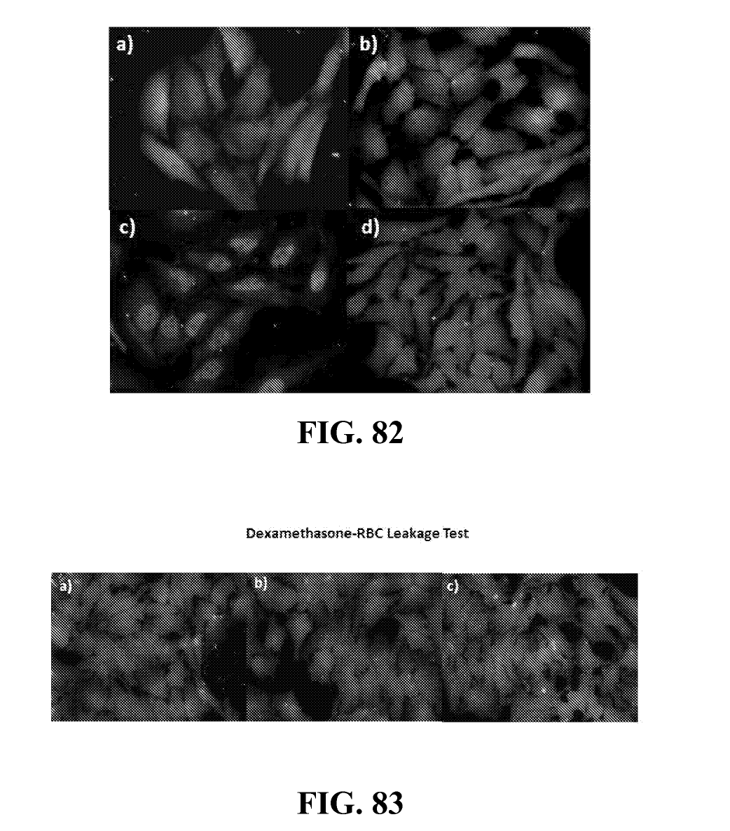

FIG. 82 shows GRα stained HeLa cells. a) Cbl-5 loaded RBCs without photolysis. b) No RBC's and no photolysis. c) Cbl-5 loaded RBCs exposed to 525 nm light for 20 min. d) No RBC's with 20 min 525 light exposure.

Treatment of HeLa cells with Cbl-5 loaded RBCs and removal pre-photolysis (Leakage Test). HeLa cells are plated in 6-well glass bottom plates (Mattek) at a density of 8.8×10⁴ cells per well and maintained at 37° C. in a humidity-controlled incubator with a 5% CO₂ atmosphere in DMEM (10% FBS, 1% Pen-Strep). The following day, cells are washed 2× with PBS, then treated with 250 μL of a suspension of Cbl-5 loaded red blood cells in L-15 media (1 μM loading volume at 5% hemocrit) or 250 μL L-15 (control cells). Cells are then incubated in the dark for 1 h at 37° C. in a humidity-controlled incubator. After the 1 hour pre-incubation, cells are washed 3×1 mL with PBS (dark room; red safe light), and 2 mL of L-15 is added to each well. The washed cells are then exposed to a green LED light source (PAR38; 500-570 nm emission; 5 mW power) or kept in the dark for 15 min at room temperature. All cells are incubated for 1 hour in a 37° C. in a humidity-controlled incubator post-photolysis. At the conclusion of the second incubation period, cells are washed 3×1 mL with PBS and then fixed with 4% PFA in PBS for 10 min at room temperature, then washed 1× with PBS and treated with 1 mL of methanol at room temperature for 5 min. Cells are subsequently washed 2×1 mL with PBS and then incubated overnight at 4° C. with rabbit anti-GRα antibody (abcam 3580) at 1:100 dilution in antibody dilution buffer (1% BSA; 0.3% Triton-X-100; PBS). Next, cells are washed with PBS (3×5 min) before incubation with anti-rabbit Alexa Fluor® 488 secondary antibody (Life Technologies A21206) at 1:500 dilution in antibody dilution buffer for 1 hour at room temperature. Cells are finally washed with PBS (3×5 min). Images are subsequently acquired with an inverted Olympus IX81 microscope equipped with a Hamamatsu C8484 camera, 40× phase contrast objective and a FITC filter cube (Semrock). Metamorph software is employed for imaging analysis.

FIG. 83 shows results of the dexamethasone-RBC leakage test. To determine if Cbl-5 is in an equilibrium with the RBCs and the cell culture, RBCs loaded with Cbl-5 were exposed to HeLa cells in a) and then removed before photolysis. GRα is not affected, indicating that dexamethasone remains on the RBC until photolysis occurs. b) Contains cells that were exposed to Cbl-5 loaded RBCs and then washed with no photolysis. c) Contains HeLa cells that were photolyzed but were not exposed to RBCs.

Treatment of HeLa cells with Cbl-5 loaded RBC at different wavelengths. HeLa cells are plated in 12-well glass bottom plates (Mattek) at a density of 2.5×10⁴ cells per well and maintained at 37° C. in a humidity-controlled incubator with a 5% CO₂ atmosphere in DMEM (10% FBS, 1% Pen-Strep). The following day, cells are washed 2× with PBS, then treated with 500 μL of a suspension of Cbl-5 loaded RBCs in L-15 media (1 μM loading volume at 5% hemocrit) or 500 μL L-15 (control cells). Cells are then either kept in the dark at 37° C. in a humidity-controlled incubator or exposed to a green LED light source (PAR38; 500-570 nm emission; 5 mW power) or a 780 nm LED array (in house creation; 7 mw) for 15 min at room temperature. All cells are incubated for 1 hour in a 37° C. in a humidity-controlled incubator post-photolysis. At the conclusion of the incubation period, cells are washed 3×1 mL with PBS and then fixed with 4% PFA in PBS for 10 min at room temperature, then washed 1× with PBS and treated with 1 mL of methanol at room temperature for 5 min. Cells are subsequently washed 2×1 mL with PBS and then incubated overnight at 4° C. with rabbit anti-GRα antibody (abcam 3580) at 1:100 dilution in antibody dilution buffer (1% BSA; 0.3% Triton-X-100; PBS). Next, cells are washed with PBS (3×5 min) before incubation with anti-rabbit Alexa Fluor® 488 secondary antibody (Life Technologies A21206) at 1:500 dilution in antibody dilution buffer for 1 h at room temperature. Cells are finally washed with PBS (3×5 min) Images are subsequently acquired with an inverted Olympus IX81 microscope equipped with a Hamamatsu C8484 camera, 40× phase contrast objective and a FITC filter cube (Semrock). Metamorph software is employed for imaging analysis.

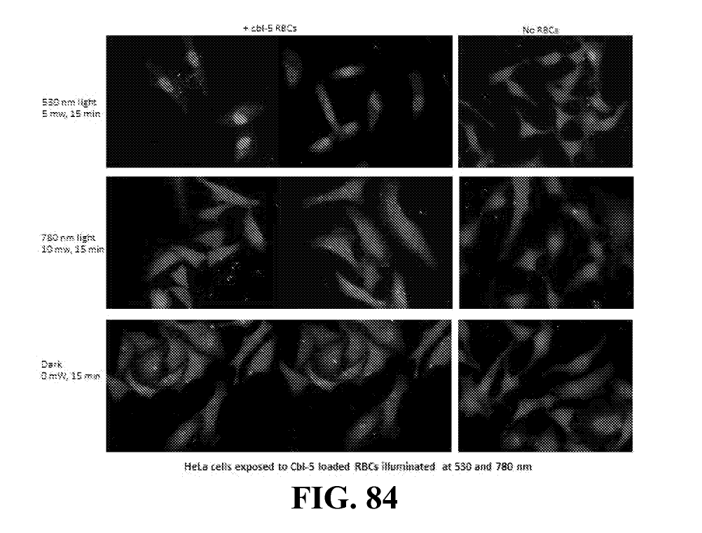

FIG. 84 shows the results of HeLa cells exposed to Cbl-5 loaded RBCs illuminated at 530 and 780 nm.

Treatment of HeLa cells with Cbl-5 and Fl-4 RBCs. HeLa cells are plated in 35 mm glass bottom dishes (Mattek) at a density of 1.1×10⁵ cells per well and maintained at 37° C. in a humidity-controlled incubator with a 5% CO₂ atmosphere in DMEM (10% FBS, 1% Pen-Strep). The following day, cells are washed 2× with PBS, then treated with 100 μL of a suspension of Cbl-5/Fl-4 loaded RBCs in L-15 media (1 μM loading volume at 5% hemocrit) or 100 μL L-15 (control cells). The cells are either kept in the dark or exposed to a 780 nm LED array (7 mW power) for 10, 20, 30, 40, or 50 min. All cells are placed in a 37° C. in a humidity-controlled incubator post-photolysis until harvest. At the conclusion of the photolysis interval, cells are washed 3×1 mL with PBS and then fixed with 4% PFA in PBS for 10 min at room temperature, then washed 1× with PBS and treated with 1 mL of methanol at room temperature for 5 min. Cells are subsequently washed 2×1 mL with PBS and then incubated overnight at 4° C. with rabbit anti-GRα antibody (abcam 3580) at 1:100 dilution in antibody dilution buffer (1% BSA; 0.3% Triton-X-100; PBS). Next, cells are washed with PBS (3×5 min) before incubation with anti-rabbit Alexa Fluor® 488 secondary antibody (Life Technologies A21206) at 1:500 dilution in antibody dilution buffer for 1 hour at room temperature. Cells are finally washed with PBS (3×5 min). Images are subsequently acquired with an inverted Olympus IX81 microscope equipped with a Hamamatsu C8484 camera, 40× phase contrast objective and a FITC filter cube (Semrock). Metamorph software is employed for imaging analysis.

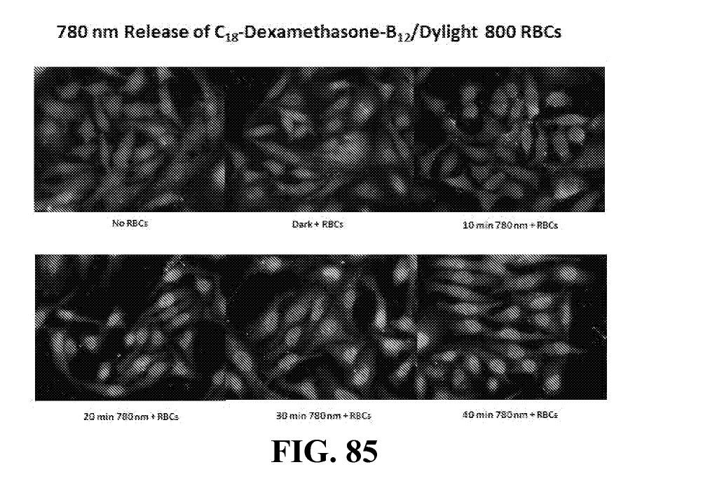

FIG. 85 shows the result of 780 nm Release of C₁₈-Dexamethasone-B₁₂/Dylight 800 RBCs.

This further example shows the hemolysis study of MTX, Colchicine and Dexamethasone.

Methods for the hemolysis Study. To a 1.5 mL Eppendorf containing a given cobalamin drug complex (Cbl-1, Cbl-3, or Cbl-5) in PBS (100 μL; 5 μM, 10 μM, 20 μM, and 40 μM) was added 100 μL RBCs in PBS (10% hemocrit). Three additional samples contained PBS treated with RBCs. Three samples contained 0.1% SDS before RBCs were added. Final concentrations were 0.5% SDS; 0 μM, 2.5 μM, 5 μM, 10 μM, and 20 μM lipidated-B₁₂-Drug. Cells were mixed by flicking before 30 min centrifugation at 300 g. Samples were re-homogenized and allowed to incubate at 4° C. overnight. Samples were pelleted at 1000 g for 5 min. 150 μL of the supernatant was plated on a 96 well plate and analyzed at 550 nm by UV-Vis. SDS samples were diluted 10-fold to be measured accurately. SDS absorbance multiplied by 10 was considered to be complete hemolysis and PBS treated blood was considered to be completely intact and the absorbance from those samples was subtracted from the background of the rest.

$$\% Hemolysis = \frac{sample absorbance}{100 \% Lysed Abs \times 10} \times 100 \%$$

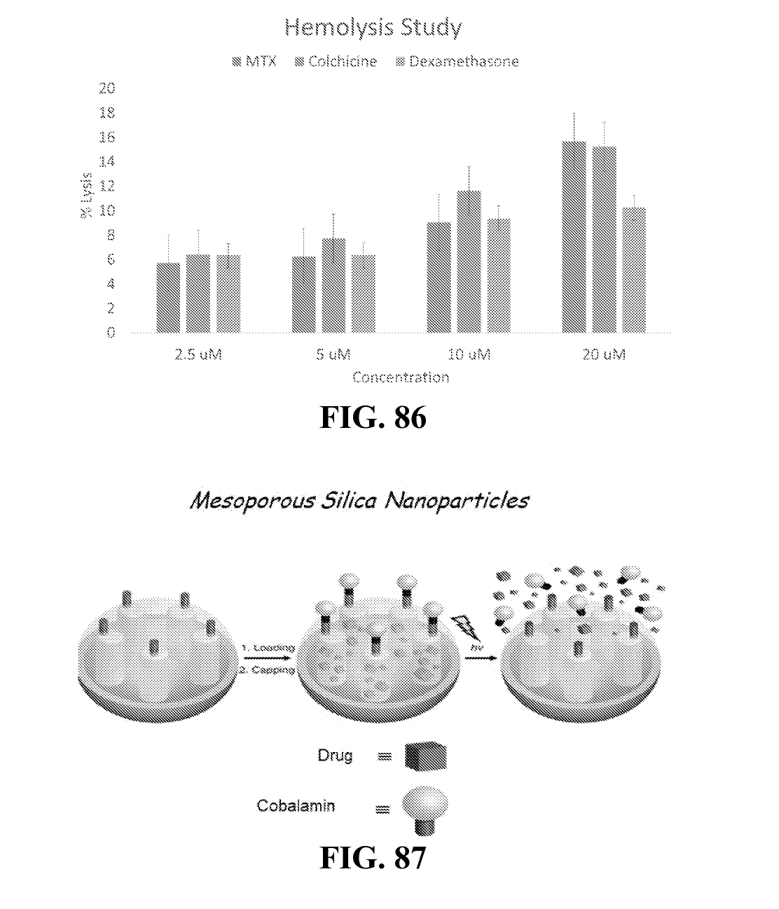

FIG. 86 shows the hemolysis study results of MTX, Colchicine and dexamethasone. Hemolysis was measured at different concentrations of each of the lipophilic drug complexes. The RBCs are stable to loading concentrations at or below 5 μM in each case.

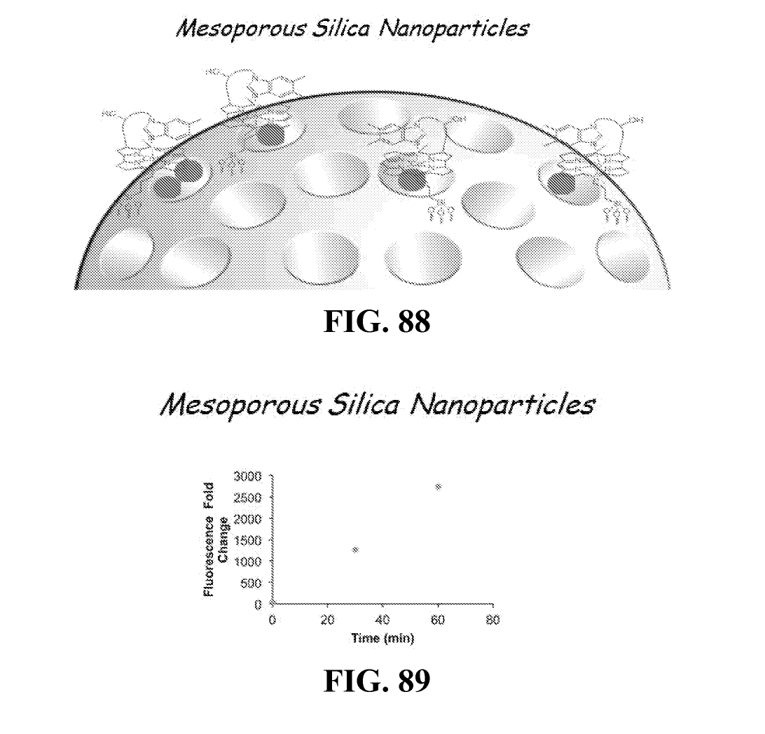

This example describes that it is not necessary to attach the drug to cobalamin in mesoporous silica nanoparticles. As shown in FIGS. 87-89, drugs are capped in the mesoporous silica nanoparticles by cobalamin-based photo-responsive constructs. FIG. 89 illustrates the release of fluorescein from cobalamin capped mesoporous silica nanoparticles (Fl-MSNP). Fluorescence intensity is relative to blank background sample. A sample was stored in the dark (5 h) then subsequently photolyzed (525 nm) for two periods (30 min) The samples were mixed (2.5 h) after each light exposure.

The exemplary drugs that can be released from nanoparticles include, but are not limited to Doxorubicin, Taxotere, Camptotechin, Various siRNAs, Cisplatin, rifampin and isoniazid, Diphtheria toxin, 5-fluorouracil, Itaconazole, cytochrome C, Insulin, cAMP, Ibuoprofen, Vancomycin, Resveratrol, Estradiol, Captopril, Aspirin, Irinotecan hydrochloride, Gentamycin, Erythromycin, Alendronate, Salvianolic acid B.

While the following terms used herein are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a fluorophore” includes a plurality of such images, and so forth.

Unless otherwise indicated, all numbers expressing quantities, properties, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter further includes any references mentioned therein, which are incorporated herein in its entirety by this reference.

### REFERENCES

Throughout this document, various references are mentioned. All such references, including those listed below, are incorporated herein by reference.
1. Majithia, V., and Geraci, S. A. (2007). Rheumatoid arthritis: diagnosis and management. Am J Med 120, 936-939.
2. Birnbaum, H., Pike, C., Kaufman, R., Marynchenko, M., Kidolezi, Y., and Cifaldi, M. (2010). Societal cost of rheumatoid arthritis patients in the US. Curr Med Res Opin 26, 77-90.
3. Hollander, J. L., Brown, E. M., Jr, Jessar, R. A., and Brown, C. Y. (1951). Hydrocortisone and cortisone injected into arthritic joints: Comparative effects of and use of hydrocortisone as a local antiarthritic agent. Journal of the American Medical Association 147, 1629-1635.
4. Mitragotri, S., and Yoo, J. W. (2011). Designing micro- and nano-particles for treating rheumatoid arthritis. Arch Pharm Res 34, 1887-1897.
5. Kukar, M., Petryna, O., and Efthimiou, P. (2009). Biological targets in the treatment of rheumatoid arthritis: a comprehensive review of current and in-development biological disease modifying anti-rheumatic drugs. Biologics 3, 443-457.
6. Huscher, D., Thiele, K., Gromnica-Ihle, E., Hein, G., Demary, W., Dreher, R., Zink, A., and Buttgereit, F. (2009). Dose-related patterns of glucocorticoid-induced side effects. Ann Rheum Dis 68, 1119-1124.
7. Baschant, U., Lane, N. E., and Tuckermann, J. (2012). The multiple facets of glucocorticoid action in rheumatoid arthritis. Nat Rev Rheumatol 8, 645-655.
8. Fiehn, C. (2010). Methotrexate transport mechanisms: the basis for targeted drug delivery and ss-folate-receptor-specific treatment. Clin Exp Rheumatol 28, S40-45.
9. Ulbrich, W., and Lamprecht, A. (2010). Targeted drug-delivery approaches by nanoparticulate carriers in the therapy of inflammatory diseases. J R Soc Interface 7 Suppl 1, S55-66.
10. Shirasu, N., Nam, S. O., and Kuroki, M. (2013). Tumor-targeted photodynamic therapy. Anticancer Res 33, 2823-2831.
11. Shamay, Y., Adar, L., Ashkenasy, G., and David, A. (2011). Light induced drug delivery into cancer cells. Biomaterials 32, 1377-1386.
12. Thompson, S., Self, A. C., and Self, C. H. (2010). Light-activated antibodies in the fight against primary and metastatic cancer. Drug Discov Today 15, 468-473.
13. Yavlovich, A., Smith, B., Gupta, K., Blumenthal, R., and Puri, A. (2010). Light-sensitive lipid-based nanoparticles for drug delivery: design principles and future considerations for biological applications. Mol Membr Biol 27, 364-381.
14. Tromberg, B. J., Shah, N., Lanning, R., Cerussi, A., Espinoza, J., Pham, T., Svaasand, L., and Butler, J. (2000). Non-invasive in vivo characterization of breast tumors using photon migration spectroscopy. Neoplasia 2, 26-40.
15. Rai, P., Mallidi, S., Zheng, X., Rahmanzadeh, R., Mir, Y., Elrington, S., Khurshid, A., and Hasan, T. (2010). Development and applications of photo-triggered theranostic agents. Adv Drug Deliv Rev 62, 1094-1124.
16. St Denis, T. G., Dai, T., Izikson, L., Astrakas, C., Anderson, R. R., Hamblin, M. R., and Tegos, G. P. (2011). All you need is light: antimicrobial photoinactivation as an evolving and emerging discovery strategy against infectious disease. Virulence 2, 509-520.
17. Vatansever, F., Ferraresi, C., de Sousa, M. V., Yin, R., Rineh, A., Sharma, S. K., and Hamblin, M. R. (2013). Can biowarfare agents be defeated with light? Virulence 4.
18. Muzykantov, V. R. (2010). Drug delivery by red blood cells: vascular carriers designed by mother nature. Expert Opin Drug Deliv 7, 403-427.
19. Getting, S. J., Kaneva, M., Bhadresa, Y., Renshaw, D., Leoni, G., Patel, H. B., Kerrigan, P. M., and Locke, I. C. (2009). Melanocortin peptide therapy for the treatment of arthritic pathologies. ScientificWorldJournal 9, 1394-1414.
20. Luger, T. A., and Brzoska, T. (2007). alpha-MSH related peptides: a new class of anti-inflammatory and immunomodulating drugs. Ann Rheum Dis 66 Suppl 3, iii52-55.
21. Yang, Y. H., Morand, E., and Leech, M. (2013). Annexin A1: potential for glucocorticoid sparing in RA. Nat Rev Rheumatol 9, 595-603.
22. Minter, R. R., Cohen, E. S., Wang, B., Liang, M., Vainshtein, I., Rees, G., Eghobamien, L., Harrison, P., Sims, D. A., Matthews, C., Wilkinson, T., Monk, P., Drinkwater, C., Fabri, L., Nash, A., McCourt, M., Jermutus, L., Roskos, L., Anderson, I. K., and Sleeman, M. A. (2013). Protein engineering and preclinical development of a GM-CSF receptor antibody for the treatment of rheumatoid arthritis. Br J Pharmacol 168, 200-211.
23. Bjordal, J. M., Lopes-Martins, R. A., Joensen, J., Couppe, C., Ljunggren, A. E., Stergioulas, A., and Johnson, M. I. (2008). A systematic review with procedural assessments and meta-analysis of low level laser therapy in lateral elbow tendinopathy (tennis elbow). BMC Musculoskelet Disord 9, 75.
24. Lee, H. M., Larson, D. R., and Lawrence, D. S. (2009). Illuminating the chemistry of life: design, synthesis, and applications of “caged” and related photoresponsive compounds. ACS Chem Biol 4, 409-427. PMCID: PMC2700207.
25. Kaplan, J. H., Forbush, B., 3rd, and Hoffman, J. F. (1978). Rapid photolytic release of adenosine 5′-triphosphate from a protected analogue: utilization by the Na:K pump of human red blood cell ghosts. Biochemistry 17, 1929-1935.
26. Aujard, I., Benbrahim, C., Gouget, M., Ruel, O., Baudin, J. B., Neveu, P., and Jullien, L. (2006). o-nitrobenzyl photolabile protecting groups with red-shifted absorption: syntheses and uncaging cross-sections for one- and two-photon excitation. Chemistry 12, 6865-6879.
27. Klan, P., Solomek, T., Bochet, C. G., Blanc, A., Givens, R., Rubina, M., Popik, V., Kostikov, A., and Wirz, J. (2013). Photoremovable protecting groups in chemistry and biology: reaction mechanisms and efficacy. Chem Rev 113, 119-191.
28. Shell, T. A., Shell, J. R., Rodgers, Z. L., and Lawrence, D. S. (2014). Tunable Visible and Near-IR Photoactivation of Light-Responsive Compounds by Using Fluorophores as Light-Capturing Antennas. Angew Chem Int Ed Engl. 53, 875-878. PMID: 24285381. PMCID in progress.
29. Pittet, M. J., and Weissleder, R. (2011). Intravital imaging. Cell 147, 983-991.
30. Luo, S., Zhang, E., Su, Y., Cheng, T., and Shi, C. (2011). A review of NIR dyes in cancer targeting and imaging. Biomaterials 32, 7127-7138.
31. Owens, S. L. (1996). Indocyanine green angiography. Br J Ophthalmol 80, 263-266.
32. East, J. M., Valentine, C. S., Kanchev, E., and Blake, G. O. (2009). Sentinel lymph node biopsy for breast cancer using methylene blue dye manifests a short learning curve among experienced surgeons: a prospective tabular cumulative sum (CUSUM) analysis. BMC Surg 9, 2.
33. Kiesslich, R., Fritsch, J., Holtmann, M., Koehler, H. H., Stolte, M., Kanzler, S., Nafe, B., Jung, M., Galle, P. R., and Neurath, M. F. (2003). Methylene blue-aided chromoendoscopy for the detection of intraepithelial neoplasia and colon cancer in ulcerative colitis. Gastroenterology 124, 880-888.
34. Hilderbrand, S. A., and Weissleder, R. (2010). Near-infrared fluorescence: application to in vivo molecular imaging. Curr Opin Chem Biol 14, 71-79.
35. Lee, H., Larson, D., and Lawrence, D. (2009). Illuminating the Chemistry of Life: Design, Synthesis, and Applications of Caged and Related Photoresponsive Compounds. ACS Chem Biol. PMCID: PMC2700207.
36. Lawrence, D. S. (2005). The preparation and in vivo applications of caged peptides and proteins. Curr Opin Chem Biol 9, 570-575.
37. Dai, Z., Dulyaninova, N. G., Kumar, S., Bresnick, A. R., and Lawrence, D. S. (2007). Visual snapshots of intracellular kinase activity at the onset of mitosis. Chem Biol 14, 1254-1260. PMCID: PMC2171364.
38. Veldhuyzen, W. F., Nguyen, Q., McMaster, G., and Lawrence, D. S. (2003). A light-activated probe of intracellular protein kinase activity. J Am Chem Soc 125, 13358-13359.
39. Wang, Q., Dai, Z., Cahill, S. M., Blumenstein, M., and Lawrence, D. S. (2006). Light-regulated sampling of protein tyrosine kinase activity. J Am Chem Soc 128, 14016-14017.
40. Wood, J. S., Koszelak, M., Liu, J., and Lawrence, D. S. (1998). A Caged Protein Kinase Inhibitor. J. Am. Chem. Soc. 120, 7145-7146.
41. Lin, W., Albanese, C., Pestell, R. G., and Lawrence, D. S. (2002). Spatially discrete, light-driven protein expression. Chem Biol 9, 1347-1353.
42. Singer, R. H., Lawrence, D. S., Ovryn, B., and Condeelis, J. (2005). Imaging of gene expression in living cells and tissues. J Biomed Opt 10, 051406.
43. Curley, K., and Lawrence, D. S. (1998). Photoactivation of a Signal Transduction Pathway in Living Cells. J Amer Chem Soc 120, 8573-8574.
44. Curley, K., and Lawrence, D. S. (1999). Caged Regulators of Signaling Pathways. Pharmacol. Ther. 82, 347-354.
45. Curley, K., and Lawrence, D. S. (1999). Light-activated proteins. Curr Opin Chem Biol 3, 84-88.
46. Ghosh, M., Ichetovkin, I., Song, X., Condeelis, J. S., and Lawrence, D. S. (2002). A new strategy for caging proteins regulated by kinases. J Am Chem Soc 124, 2440-2441.
47. Ghosh, M., Song, X., Mouneimne, M., Sidani, M., Lawrence, D. S., and Condeelis, J. S. (2004). Cofilin promotes actin polymerization and defines the direction of cell motility. Science 304, 743-736.
48. Dai, Z., Dulyaninova, N. G., Kumar, S., Bresnick, A. R., and Lawrence, D. S. (2007). Visual snapshots of intracellular kinase activity at the onset of mitosis. Chem. Biol. 14, 1254-1260. PMCID: PMC2171364.
49. Larson, D. R., Fritzsch, C., Sun, L., Meng, X., Lawrence, D. S., and Singer, R. H. (2013). Direct observation of frequency modulated transcription in single cells using light activation. Elife 2, e00750. PMCID: PMC3780543.
50. Gug, S., Charon, S., Specht, A., Alarcon, K., Ogden, D., Zietz, B., Leonard, J., Haacke, S., Bolze, F., Nicoud, J. F., and Goeldner, M. (2008). Photolabile glutamate protecting group with high one- and two-photon uncaging efficiencies. Chembiochem 9, 1303-1307.
51. Bort, G., Gallavardin, T., Ogden, D., and Dalko, P. I. (2013). From one-photon to two-photon probes: “caged” compounds, actuators, and photoswitches. Angew Chem Int Ed Engl 52, 4526-4537.
52. Barker, H. A., Weissbach, H., and Smyth, R. D. (1958). A Coenzyme Containing Pseudovitamin B(12). Proc Natl Acad Sci USA 44, 1093-1097.
53. Dolphin, D., Johnson, A. W., and Rodrigo, R. (1964). Some Reactions of the Vitamin B12 Coenzyme and Its Alkyl Analogues. Ann N Y Acad Sci 112, 590-600.
54. Taylor, R. T., Smucker, L., Hanna, M. L., and Gill, J. (1973). Aerobic photolysis of alkylcobalamins: quantum yields and light-action spectra. Arch Biochem Biophys 156, 521-533.
55. Halpern, J., Kim, S.-H., and Leung, T. W. (1984). Cobalt-carbon bond dissociation energy of coenzyme B12. J. Am. Chem. Soc. 106, 8317-8319.
56. Kozlowski, P. M., Kumar, M., Piecuch, P., Li, W., Bauman, N. P., Hansen, J. A., Lodowski, P., and Jaworska, M. (2012). The Cobalt-Methyl Bond Dissociation in Methylcobalamin: New Benchmark Analysis Based on Density Functional Theory and Completely Renormalized Coupled-Cluster Calculations. Journal of Chemical Theory and Computation 8, 1870-1894.
57. Bagnato, J. D., Eilers, A. L., Horton, R. A., and Grissom, C. B. (2004). Synthesis and characterization of a cobalamin-colchicine conjugate as a novel tumor-targeted cytotoxin. J Org Chem 69, 8987-8996.
58. Gupta, Y., Kohli, D. V., and Jain, S. K. (2008). Vitamin B12-mediated transport: a potential tool for tumor targeting of antineoplastic drugs and imaging agents. Crit Rev Ther Drug Carrier Syst 25, 347-379.
59. Kamkaew, A., Lim, S. H., Lee, H. B., Kiew, L. V., Chung, L. Y., and Burgess, K. (2013). BODIPY dyes in photodynamic therapy. Chem Soc Rev 42, 77-88.
60. Awuah, S. G., and You, Y. (2012). Boron dipyrromethene (BODIPY)-based photosensitizers for photodynamic therapy. RSC Advances 2, 11169-11183.
61. Oishi, A., Makita, N., Sato, J., and Iiri, T. (2012). Regulation of RhoA signaling by the cAMP-dependent phosphorylation of RhoGDIalpha. J Biol Chem 287, 38705-38715.
62. Patil, R. R., Guhagarkar, S. A., and Devarajan, P. V. (2008). Engineered nanocarriers of doxorubicin: a current update. Crit Rev Ther Drug Carrier Syst 25, 1-61.
63. Volkova, M., and Russell, R., 3rd (2011). Anthracycline cardiotoxicity: prevalence, pathogenesis and treatment. Curr Cardiol Rev 7, 214-220.
64. Kuhn, H. J., Braslaysky, S. E., and Schmidt, R. (1989). Chemical actinometry. Pure Appl. Chem. 61, 187-210.
65. Majumdar, S., Anderson, M. E., Xu, C. R., Yakovleva, T. V., Gu, L. C., Malefyt, T. R., and Siahaan, T. J. (2012). Methotrexate (MTX)-cIBR conjugate for targeting MTX to leukocytes: conjugate stability and in vivo efficacy in suppressing rheumatoid arthritis. J Pharm Sci 101, 3275-3291.
66. Wang, D., Miller, S. C., Liu, X. M., Anderson, B., Wang, X. S., and Goldring, S. R. (2007). Novel dexamethasone-HPMA copolymer conjugate and its potential application in treatment of rheumatoid arthritis. Arthritis Res Ther 9, R2.
67. Everts, M., Kok, R. J., Asgeirsdottir, S. A., Melgert, B. N., Moolenaar, T. J., Koning, G. A., van Luyn, M. J., Meijer, D. K., and Molema, G. (2002). Selective intracellular delivery of dexamethasone into activated endothelial cells using an E-selectin-directed immunoconjugate. J Immunol 168, 883-889.
68. Heath, T. D., Lopez, N. G., Piper, J. R., Montgomery, J. A., Stern, W. H., and Papahadjopoulos, D. (1986). Liposome-mediated delivery of pteridine antifolates to cells in vitro: potency of methotrexate, and its alpha and gamma substituents. Biochim Biophys Acta 862, 72-80.
69. Rosowsky, A., Bader, H., Radike-Smith, M., Cucchi, C. A., Wick, M. M., and Freisheim, J. H. (1986). Methotrexate analogues. 28. Synthesis and biological evaluation of new gamma-monoamides of aminopterin and methotrexate. J Med Chem 29, 1703-1709.
70. Piper, J. R., Montgomery, J. A., Sirotnak, F. M., and Chello, P. L. (1982). Syntheses of alpha- and gamma-substituted amides, peptides, and esters of methotrexate and their evaluation as inhibitors of folate metabolism. J Med Chem 25, 182-187.
71. Rosowsky, A., Forsch, R., Uren, J., and Wick, M. (1981). Methotrexate analogues. 14. Synthesis of new gamma-substituted derivatives as dihydrofolate reductase inhibitors and potential anticancer agents. J Med Chem 24, 1450-1455.
72. Szeto, D. W., Cheng, Y. C., Rosowsky, A., Yu, C. S., Modest, E. J., Piper, J. R., Temple, C., Jr., Elliott, R. D., Rose, J. D., and Montgomery, J. A. (1979). Human thymidylate synthetase—III. Effects of methotrexate and folate analogs. Biochem Pharmacol 28, 2633-2637.
73. Markovic, B. D., Vladimirov, S. M., Cudina, O. A., Odovic, J. V., and Karljikovic-Rajic, K. D. (2012). A PAMPA assay as fast predictive model of passive human skin permeability of new synthesized corticosteroid C-21 esters. Molecules 17, 480-491.
74. Civiale, C., Bucaria, F., Piazza, S., Peri, O., Miano, F., and Enea, V. (2004). Ocular permeability screening of dexamethasone esters through combined cellular and tissue systems. J Ocul Pharmacol Ther 20, 75-84.
75. Samtani, M. N., Lohle, M., Grant, A., Nathanielsz, P. W., and Jusko, W. J. (2005). Betamethasone pharmacokinetics after two prodrug formulations in sheep: implications for antenatal corticosteroid use. Drug Metab Dispos 33, 1124-1130.
76. Bohm, M., and Grassel, S. (2012). Role of proopiomelanocortin-derived peptides and their receptors in the osteoarticular system: from basic to translational research. Endocr Rev 33, 623-651.
77. Pezacka, E., Green, R., and Jacobsen, D. W. (1990). Glutathionylcobalamin as an intermediate in the formation of cobalamin coenzymes. Biochem Biophys Res Commun 169, 443-450.
78. Brasch, N. E., Hsu, T.-L. C., Doll, K. M., and Finke, R. G. (1999). Synthesis and characterization of isolable thiolatocobalamin complexes relevant to coenzyme B₁₂-dependent ribonucleoside triphosphate reductase J. Inorg. Biochem. 76, 197-209.
79. Suarez-Moreira, E., Hannibal, L., Smith, C. A., Chavez, R. A., Jacobsen, D. W., and Brasch, N. E. (2006). A simple, convenient method to synthesize cobalamins: synthesis of homocysteinylcobalamin, N-acetylcysteinylcobalamin, 2-N-acetylamino-2-carbomethoxyethanethiolatocobalamin, sulfitocobalamin and nitrocobalamin. Dalton T., 5269-5277.
80. Tahara, K., Matsuzaki, A., Masuko, T., Kikuchi, J., and Hisaeda, Y. (2013). Synthesis, characterization, Co—S bond reactivity of a vitamin B12 model complex having pentafluorophenylthiolate as an axial ligand. Dalton Trans 42, 6410-6416.
81. Peng, J., Tang, K. C., McLoughlin, K., Yang, Y., Forgach, D., and Sension, R. J. (2010). Ultrafast excited-state dynamics and photolysis in base-off B12 coenzymes and analogues: absence of the trans-nitrogenous ligand opens a channel for rapid nonradiative decay. J Phys Chem B 114, 12398-12405.
82. Lee, T. R., and Lawrence, D. S. (1999). Acquisition of high-affinity, SH2-targeted ligands via a spatially focused library. J Med Chem 42, 784-787.
83. Lee, T. R., and Lawrence, D. S. (2000). SH2-directed ligands of the Lck tyrosine kinase. J Med Chem 43, 1173-1179.
84. Yeh, R. H., Lee, T. R., and Lawrence, D. S. (2001). From consensus sequence peptide to high affinity ligand, a “library scan” strategy. J Biol Chem 276, 12235-12240.
85. Best, M. D. (2009). Click chemistry and bioorthogonal reactions: unprecedented selectivity in the labeling of biological molecules. Biochemistry 48, 6571-6584.
86. Kolb, H. C., Finn, M. G., and Sharpless, K. B. (2001). Click Chemistry: Diverse Chemical Function from a Few Good Reactions. Angew Chem Int Ed Engl 40, 2004-2021.
87. Nguyen, L. T., Oien, N. P., Allbritton, N. L., and Lawrence, D. S. (2013). Lipid pools as photolabile “protecting groups”: design of light-activatable bioagents. Angew Chem Int Ed Engl 52, 9936-9939. PMCID: PMC3840492.
88. Muzykantov, V. R. (2013). Drug delivery carriers on the fringes: natural red blood cells versus synthetic multilayered capsules. Expert Opin Drug Deliv 10, 1-4.
89. Smith, W., and Lawrence, D. S. unpublished results.
90. Leschke, C., Storm, R., Breitweg-Lehmann, E., Exner, T., Nurnberg, B., and Schunack, W. (1997). Alkyl-substituted amino acid amides and analogous di- and triamines: new non-peptide G protein activators. J Med Chem 40, 3130-3139.
91. Krantz, A. (1997). Red cell-mediated therapy: opportunities and challenges. Blood Cells Mol Dis 23, 58-68.
92. Rossi, L., Serafini, S., Pierige, F., Castro, M., Ambrosini, M. I., Knafelz, D., Damonte, G., Annese, V., Latiano, A., Bossa, F., and Magnani, M. (2006). Erythrocytes as a controlled drug delivery system: clinical evidences. J Control Release 116, e43-45.
93. Biagiotti, S., Paoletti, M. F., Fraternale, A., Rossi, L., and Magnani, M. (2011). Drug delivery by red blood cells. IUBMB Life 63, 621-631.
94. Rossi, L., Castro, M., D'Orio, F., Damonte, G., Serafini, S., Bigi, L., Panzani, I., Novelli, G., Dallapiccola, B., Panunzi, S., Di Carlo, P., Bella, S., and Magnani, M. (2004). Low doses of dexamethasone constantly delivered by autologous erythrocytes slow the progression of lung disease in cystic fibrosis patients. Blood Cells Mol Dis 33, 57-63.
95. Intra-Erythrocyte Dexamethasone Sodium Phosphate in Ataxia Teleangiectasia Patients (IEDAT01). http://clinicaltrials.gov/show/NCT01255358
96. Bossa, F., Latiano, A., Rossi, L., Magnani, M., Palmieri, O., Dallapiccola, B., Serafini, S., Damonte, G., De Santo, E., Andriulli, A., and Annese, V. (2008). Erythrocyte-mediated delivery of dexamethasone in patients with mild-to-moderate ulcerative colitis, refractory to mesalamine: a randomized, controlled study. Am J Gastroenterol 103, 2509-2516.
97. Castro, M., Rossi, L., Papadatou, B., Bracci, F., Knafelz, D., Ambrosini, M. I., Calce, A., Serafini, S., Isacchi, G., D'Orio, F., Mambrini, G., and Magnani, M. (2007). Long-term treatment with autologous red blood cells loaded with dexamethasone 21-phosphate in pediatric patients affected by steroid-dependent Crohn disease. J Pediatr Gastroenterol Nutr 44, 423-426.
98. Vivero-Escoto, J. L., Slowing, I I, Trewyn, B. G., and Lin, V. S. (2010). Mesoporous silica nanoparticles for intracellular controlled drug delivery. Small 6, 1952-1967.
99. Li, Z., Barnes, J. C., Bosoy, A., Stoddart, J. F., and Zink, J. I. (2012). Mesoporous silica nanoparticles in biomedical applications. Chem Soc Rev 41, 2590-2605.
100. Coll, C., Bernardos, A., Martinez-Manez, R., and Sancenon, F. (2013). Gated silica mesoporous supports for controlled release and signaling applications. Acc Chem Res 46, 339-349.
101. Croissant, J., Maynadier, M., Gallud, A., Peindy N'dongo, H., Nyalosaso, J. L., Derrien, G., Charnay, C., Durand, J. O., Raehm, L., Serein-Spirau, F., Cheminet, N., Jarrosson, T., Mongin, O., Blanchard-Desce, M., Gary-Bobo, M., Garcia, M., Lu, J., Tamanoi, F., Tarn, D., Guardado-Alvarez, T. M., and Zink, J. I. (2013). Two-photon-triggered drug delivery in cancer cells using nanoimpellers. Angew Chem Int Ed Engl 52, 13813-13817.
102. Mal, N. K., Fujiwara, M., and Tanaka, Y. (2003). Photocontrolled reversible release of guest molecules from coumarin-modified mesoporous silica. Nature 421, 350-353.
103. Wan, X., Liu, T., Hu, J., and Liu, S. (2013). Photo-degradable, protein-polyelectrolyte complex-coated, mesoporous silica nanoparticles for controlled co-release of protein and model drugs. Macromol Rapid Commun 34, 341-347.
104. Popat, A., Hartono, S. B., Stahr, F., Liu, J., Qiao, S. Z., and Qing Max Lu, G. (2011). Mesoporous silica nanoparticles for bioadsorption, enzyme immobilisation, and delivery carriers. Nanoscale 3, 2801-2818.
105. Catania, A., Gatti, S., Colombo, G., and Lipton, J. M. (2004). Targeting melanocortin receptors as a novel strategy to control inflammation. Pharmacol Rev 56, 1-29.
106. Bouis, D., Hospers, G. A., Meijer, C., Molema, G., and Mulder, N. H. (2001). Endothelium in vitro: a review of human vascular endothelial cell lines for blood vessel-related research. Angiogenesis 4, 91-102.
107. Zvaifler, N.J., Boyle, D., and Firestein, G. S. (1994). Early synovitis—synoviocytes and mononuclear cells. Semin Arthritis Rheum 23, 11-16.
108. Kang, Y. M., Zhang, X., Wagner, U. G., Yang, H., Beckenbaugh, R. D., Kurtin, P. J., Goronzy, J. J., and Weyand, C. M. (2002). CD8 T cells are required for the formation of ectopic germinal centers in rheumatoid synovitis. J Exp Med 195, 1325-1336.
109. Chan, E. S., and Cronstein, B. N. (2010). Methotrexate—how does it really work? Nat Rev Rheumatol 6, 175-178.
110. Chen, W., Lee, J. Y., and Hsieh, W. C. (2002). Effects of dexamethasone and sex hormones on cytokine-induced cellular adhesion molecule expression in human endothelial cells. Eur J Dermatol 12, 445-448.
111. Nehme, A., and Edelman, J. (2008). Dexamethasone inhibits high glucose-, TNF-alpha-, and IL-1beta-induced secretion of inflammatory and angiogenic mediators from retinal microvascular pericytes. Invest Ophthalmol Vis Sci 49, 2030-2038.
112. Joyce, D. A., Steer, J. H., and Abraham, L. J. (1997). Glucocorticoid modulation of human monocyte/macrophage function: control of TNF-alpha secretion. Inflamm Res 46, 447-451.
113. Peshavariya, H. M., Taylor, C. J., Goh, C., Liu, G. S., Jiang, F., Chan, E. C., and Dusting, G. J. (2013). Annexin peptide Ac2-26 suppresses TNFalpha-induced inflammatory responses via inhibition of Racl-dependent NADPH oxidase in human endothelial cells. PLoS One 8, e60790.
114. Morabito, L., Montesinos, M. C., Schreibman, D. M., Balter, L., Thompson, L. F., Resta, R., Carlin, G., Huie, M. A., and Cronstein, B. N. (1998). Methotrexate and sulfasalazine promote adenosine release by a mechanism that requires ecto-5′-nucleotidase-mediated conversion of adenine nucleotides. J Clin Invest 101, 295-300.
115. Linden, J., and Cekic, C. (2012). Regulation of lymphocyte function by adenosine. Arterioscler Thromb Vasc Biol 32, 2097-2103.
116. Wright, G. P., Stauss, H. J., and Ehrenstein, M. R. (2011). Therapeutic potential of Tregs to treat rheumatoid arthritis. Semin Immunol 23, 195-201.
117. Taylor, A. W., and Lee, D. J. (2011). The alpha-melanocyte stimulating hormone induces conversion of effector T cells into treg cells. J Transplant 2011, 246856.
118. Muller, W. A., and Luscinskas, F. W. (2008). Assays of transendothelial migration in vitro. Methods Enzymol 443, 155-176.
119. Shulman, Z., and Alon, R. (2009). Chapter 14. Real-time in vitro assays for studying the role of chemokines in lymphocyte transendothelial migration under physiologic flow conditions. Methods Enzymol 461, 311-332.
120. Grisar, J., Aringer, M., Koller, M. D., Stummvoll, G. H., Eselbock, D., Zwolfer, B., Steiner, C. W., Zierhut, B., Wagner, L., Pietschmann, P., and Smolen, J. S. (2004). Leflunomide inhibits transendothelial migration of peripheral blood mononuclear cells. Ann Rheum Dis 63, 1632-1637.
121. Yang, Y. H., Rajaiah, R., Ruoslahti, E., and Moudgil, K. D. (2011). Peptides targeting inflamed synovial vasculature attenuate autoimmune arthritis. Proc Natl Acad Sci USA 108, 12857-12862.
122. Wythe, S. E., DiCara, D., Taher, T. E., Finucane, C. M., Jones, R., Bombardieri, M., Man, Y. K., Nissim, A., Mather, S. J., Chernajovsky, Y., and Pitzalis, C. (2013). Targeted delivery of cytokine therapy to rheumatoid tissue by a synovial targeting peptide. Ann Rheum Dis 72, 129-135.
123. Lee, L., Buckley, C., Blades, M. C., Panayi, G., George, A. J., and Pitzalis, C. (2002). Identification of synovium-specific homing peptides by in vivo phage display selection. Arthritis Rheum 46, 2109-2120.
124. Lin, H. B., Garcia-Echeverria, C., Asakura, S., Sun, W., Mosher, D. F., and Cooper, S. L. (1992). Endothelial cell adhesion on polyurethanes containing covalently attached RGD-peptides. Biomaterials 13, 905-914.
125. Fens, M. H., Storm, G., Pelgrim, R. C., Ultee, A., Byrne, A. T., Gaillard, C. A., van Solinge, W. W., and Schiffelers, R. M. (2010). Erythrophagocytosis by angiogenic endothelial cells is enhanced by loss of erythrocyte deformability. Exp Hematol 38, 282-291.
126. Neogen Corporation. http://www.neogen.com/Toxicology/pdf/Catalogs/Forensic_Catalog_13. pdf
127. Alpha Labs. http://www.alphalabs.co.uk/product-information/diagnostic-products/clinical-chemistry/methotrexate.aspx
128. (a) Lee, H. M.; Larson, D. R.; Lawrence, D. S. ACS Chem. Biol. 2009, 4, 409-27. (b) Brieke, C.; Rohrbach, F.; Gottschalk, A.; Mayer, G. Angew. Chem. Int. Ed. Engl. 2012, 51, 8446-76. (c) Klan, P.; Solomek, T.; Bochet, C. G.; Blanc, A.; Givens, R.; Rubina, M.; Popk, V.; Kostikov, A.; Wirz, J. Chem. Rev. 2013, 113, 119-91.
129. Tromberg, B. J.; Shah, N.; Lanning, R.; Espinoza, J.; Pham, T.; Svaasand, L.; Butler, J. Neoplasia 2000, 2, 26-40.
130. (a) Goguen, B. N.; Aemissegger, A.; Imperiali, B. J. Am. Chem. Soc. 2011, 133, 11038-41. (b) Hagen, V., Dekowski, B.; Nache, V.; Schmidt, R.; Geibler, D.; Lorenz, D.; Eichhorst, J.; Keller, S.; Kaneko, H.; Benndorf, K.; Wiesner, B. Angew. Chem. Int. Ed. Engl. 2005, 44, 7887-91. (c) Kantevari, S.; Matsuzaki, M.; Kanemoto, Y.; Kasai, H.; Ellis-Davies, G. C. Nat. Methods 2010, 7, 123-5. (d) Menge, C.; Heckel, A. Org. Lett. 2011, 13, 4620-3. (e) Priestman, M. A.; Sun, L.; Lawrence, D. S. ACS Chem. Biol. 2011, 6, 377-84.
131. Bort, G.; Gallavardin, T.; Ogden, D.; Dalko, P. I. Angew. Chem. Intl. Ed. Engl. 2013, doi: 10.1002/anie.201204203. [Epub ahead of print].
132. Priestman, M. A.; Shell, T. A.; Sun, L.; Lee, H.-M.; Lawrence, D. S. Angew. Chem. Int. Ed. Engl. 2012, 51, 7684-7.
133. Taylor, R. T.; Smucker, L.; Hanna, M. L.; Gill, J. Arch. Biochem. Biophys. 1973, 156, 521-3.
134. (a) Lee, M.; Grissom, C. B. Org. Lett. 2009, 11, 2499-502. (b) Smeltzer, C. C.; Cannon, M. J.; Pinson, P. R.; Munger, J. D., Jr.; West, F. G.; Grissom, C. B. Org. Lett. 2001, 3, 799-801. (c) Jacobsen, D. W. Methods Enzym. 1980, 67, 12-9. (d) Rosendahl, M. S.; Omann, G. M.; Leonard, N. J. Proc. Nat. Acad. Sci. USA 1982, 79, 3480-4. (e) Jacobsen, D. W.; Holland, R. J.; Montejano, Y.; Huennekens, F. M. J. Inorg. Biochem. 1979, 10, 53-65.
135. (a) Halpern, J.; Kim, S.-H.; Leung, T. W. J. Am. Chem. Soc. 1984, 106, 8317-9. (b) Kozlowski, P. M.; Kumar, M.; Piecuch, P.; Li, W.; Bauman, N. P.; Hansen, J. A.; Lodowski, P.; Jaworska, M.; J. Chem. Theory Comput. 2012, 8, 1870-94 and references cited therein.
136. Schwartz, P. A.; Frey, P. A. Biochemistry 2007, 46, 7284-92.
137. Lee, H. M.; Priestman, M. A.; Lawrence, D. S. J. Am. Chem. Soc. 2010, 132, 1446-7.
138. (a) Bagnato, J. D.; Eilers, A. L.; Horton, R. A.; Grissom, C. B. J. Org. Chem. 2004, 69, 8987-96. (b) Gupta, Y.; Kohli, D. V.; Jain, S. K. Crit. Rev. Ther. Drug. Carrier Syst. 2008, 25, 347-78.
139. (a) Kamkaew, A.; Lim, S. H.; Lee, H. B.; Kiew, L. V.; Chung, L. Y.; Burgess, K. Chem. Soc. Rev. 2013, 42, 77-88. (b) Awuah, S. G.; You, Y. RSC Advances 2012, 2, 11169-83. (c) Lim, S. H.; Thivierge, C.; Nowak-Sliwinska, P.; Han, J.; van den Bergh, H.; Wagnières, G.; Burgess, K.; Lee, H. B. J. Med. Chem. 2010, 53, 2865-74.
140. (a) Bochet, C. G. Angew. Chem. Int. Ed. Engl. 2001, 40, 2071-3. (b) Kotzur, N.; Briand, B.; Beyermann, M.; Hagen, V. J. Am. Chem. Soc. 2009, 131, 16927-31.
141. Al-Saigh, H. Y.; Kemp, T. J. J. Chem. Soc. Perkin Trans. 2 1983, 615-9.
142. Agostinis, P., et al. CA Cancer J. Clin. 2011, 61, 250-281.
143. Allison, R. R.; Bagnato, V. S.; Sibata, C. H. Future Oncol. 2010, 6(6), 929-940.
144. Smeltzer, C. C.; Cannon, M. J.; Pinson, P. R.; Munger, J. D., Jr.; West, F. G.; Grissom, C. B Org. Lett., 2001, 3, 799-801.
145. Priestman, M. A.; Shell, T. A.; Sun, L.; Lee, H.-M.; Lawrence, D. S. Angew. Chem. 2012, 124, 7804-7807; Angew. Chem. Int. Ed. 2012, 51, 7684-7687.
146. The formula and exact mass of Atto725, carboxylic acid have not been reported.
147. The formula and exact mass of Dylight800, carboxylic acid have not been reported.
148. The formula and an accurate mass of Alexa700, carboxylic acid have not been reported.
149. Taylor, R. T.; Smucker, L.; Hanna, M. L.; Gill, J. Arch. Biochem. Biophys. 1973, 156, 521-533.

1. A composition, comprising:
a photolabile molecule;
a bioactive agent that is appended to the photolabile molecule, wherein the bioactive agent is released when exposed to a certain wavelength of light; and
a fluorophore that is selected to be activated at the certain wavelength of light such that the bioactive agent is released.

2. The composition of claim 1, wherein the photolabile molecule is cobalamin or a derivative or analogue thereof.

3. The composition of claim 1, wherein the photolabile molecule is an alkylcobalamin.

4. The composition of claim 1, further comprising a second photolabile molecule; a second bioactive agent that is appended to the photolabile molecule, wherein the second bioactive agent is released when exposed to a second wavelength of light; and a second fluorophore that is selected to be activated at the second wavelength of light such that the second bioactive agent is released.

5-7. (canceled)

8. The composition of claim 1, wherein the bioactive agent comprises an anti-rheumatoid arthritis agent or an agent for the treatment of glioblastoma.

9. The composition of claim 1, wherein the fluorophore is appended to at least one of a cobalt center of the cobalamin and a ribose 5′-OH of the cobalamin.

10. The composition of claim 1, further comprising a linker disposed between the photolabile molecule and the first active agent.

11. The composition of claim 10, wherein the linker comprises an alkyl, an aryl, an amino, a thioether, a carboxamide, an ester, an ether, or a combination thereof.

12. The composition of claim 10, wherein the linker comprises a propylamine, an ethylenediamine or a combination or derivative thereof.

13. (canceled)

14. The composition of claim 1, wherein the light comprises a wavelength of about 500 nm to about 1000 nm.

15-16. (canceled)

17. A method of treating a disease, comprising:
administering an effective amount of the composition of claim 1 to a subject at an administration site; and
then exposing the administration site to the certain wavelength of light.

18. The method of claim 17, wherein the photolabile molecule is a cobalamin or a derivative or analogue thereof.

19. The method of claim 18, wherein the cobalamin is an alkylcobalamin.

20. The method of claim 17, wherein the composition further comprises a second photolabile molecule; a second bioactive agent that is appended to the photolabile molecule, wherein the second bioactive agent is released when exposed to a second wavelength of light; and a second fluorophore that is selected to be activated at the second wavelength of light such that the second bioactive agent is released; and the method further comprising exposing the administration site to the second wavelength of light.

21-27. (canceled)

28. The method of claim 17, wherein the light comprises a wavelength of about 500 nm to about 1000 nm.

29-30. (canceled)

31. The method of claim 17, wherein the disease is rheumatoid arthritis or glioblastoma.

32-34. (canceled)

35. The composition of claim 1, and further comprising a lipid, wherein the bioactive agent and the lipid are appended to the photolabile molecule.

36-37. (canceled)

38. The composition of claim 35, and further comprising at least one membrane layer, wherein the composition is incorporated in the at least one membrane layer.

39. The composition of claim 38, wherein the flurophore is incorporated in the at least one membrane layer.

40. The composition of claim 38, wherein the membrane is the membrane of a red blood cell.

41-43. (canceled)
